(19) United States
(12) Patent Application Publication
(10) Pub. No.: US 2020/0129669 A1
(43) Pub. Date: Apr. 30, 2020

(54) BIORESORBABLE MAGNESIUM-BASED SPONGE AND FOAM MATERIALS, METHODS AND DEVICES

(71) Applicant: The Board of Trustees of the University of Illinois, Urbana, IL (US)

(72) Inventors: Jean Paul ALLAIN, Champaign, IL (US); Akshath SHETTY, Mumbai (IN); Juan Fernando RAMIREZ PATINO, Medellin (CO); Viviana POSADA, Medellin (CO); Patricia FERNANDEZ MORALES, Medellin (CO)

(73) Assignee: The Board of Trustees of the University of Illinois, Urbana, IL (US)

(21) Appl. No.: 16/592,125

(22) Filed: Oct. 3, 2019

Related U.S. Application Data
(63) Continuation of application No. PCT/US18/26574, filed on Apr. 6, 2018.
(60) Provisional application No. 62/483,105, filed on Apr. 7, 2017.
(60) Provisional application No. 62/556,120, filed on Sep. 8, 2017.

(51) Int. Cl. A61L 27/56; A61L 27/04; A61L 27/58; A61L 27/54

(52) U.S. Cl. CPC A61L 27/56; A61L 27/047; A61L 27/58; A61L 2300/41; A61L 2400/12; A61L 2300/404; A61L 27/54

(57) ABSTRACT

Provided herein are magnesium-based sponges and foams and methods for surface modification to enhance bioactivity. The magnesium-based sponges and foams may be useful as tissue or bone grafts which promote cellular adhesion and osseointegration and conduction, as well as various other biological functions including, for example, antibacterial properties, hydrophobicity or hydrophilicity and the ability to modulate immune response. The described sponges and foams have precise mechanical properties which are specifically designed for enhanced integration with surrounding tissue. The described magnesium-based materials are bioresorbable, allowing for the gradual, safe absorption of the material when exposed to bodily fluids.

### CROSS-REFERENCE TO RELATED APPLICATIONS

This application claims the benefit of and priority to U.S. Provisional Application No. 62/483,105, filed Apr. 7, 2017 and U.S. Provisional Application No. 62/556,120, filed Sep. 8, 2017 which are each hereby incorporated by reference in their entirety to the extent not inconsistent herewith.

### BACKGROUND OF INVENTION

Magnesium and magnesium alloys are widely recognized as advantageous materials for biological implants, including bone implants, tissue scaffolds and venous implants. Magnesium is typically preferred for applications in which the implant is temporary, as magnesium naturally resorbs into solution when contacted with a biological fluid and magnesium is common in the human body. Thus, magnesium implants safely resorb over a period of time and do not require surgical removal after the implant has served its purpose. Additionally, magnesium is attractive as an implant material for its mechanical properties. For example, implants may be tailored to closely mimic the tissue in which the implant is interacting, such as bone, providing more successful implantation, faster healing and increased integration between the implant and the host tissue.

A major drawback of magnesium implants is that the bioresorption of the implant occurs heterogeneously and rapidly, which can compromise the mechanical strength of the implant. Additionally, the bioresorption process may generate hydrogen gas. While small amounts of hydrogen gas can be naturally removed by the body, larger quantities generated by rapid bioresorption can lead to inflammation and necrosis.

Plasma has also been used to alter chemical and mechanical properties of magnesium implants. However, known methods of plasma treatment (e.g. kinetic roughening) are imprecise and provide little control over the plasma surface interaction. By providing precise porous and/or nanopatterned regions to the implant, including tailored to the specific application of the implant, bioresorption and biomechanical stresses may be reduced and cell adhesion increased, resulting in longer implant life, faster patient recovery times and reduced risk of implant tissue damage, complications and infections.

It can be seen from the foregoing that there remains a need in the art for bioresorbable magnesium-based sponges and foams with designed mechanical properties, surface chemistries, nanofeatures, crystallographic structures and/or morphologies which provide enhanced bioactivity and functionality over previously described medical implants, for example, bone and tissue grafts. Further, methods of surface modification of the described sponges and foams may increase utility as medical implants.

### SUMMARY OF THE INVENTION

Provided herein are magnesium-based sponges and foams and methods for surface modification to enhance bioactivity. The magnesium-based sponges and foams may be useful as tissue or bone grafts which promote cellular adhesion and osseointegration and conduction, as well as various other biological functions including, for example, antibacterial properties, hydrophobicity or hydrophilicity and the ability to modulate immune response. The described sponges and foams have precise mechanical properties which are specifically designed for enhanced integration with surrounding tissue. The described magnesium-based materials are bioresorbable, allowing for the gradual, safe absorption of the material when exposed to bodily fluids.

The provided compositions are modified to provide controlled bioresorption profiles and/or alter biological properties or functions. The surface of the composition may be modified by independently controlling parameters (e.g. incident angle, fluence, flux, energy, species, etc.) of one or more directed energetic particle beams, providing more control and increased bioactivity over conventional kinetic roughing techniques. The provided methods also allow for specific modification of chemical composition, for instance, accurate creation of one or more alloys different from adjacent domains or the original underlying substrate, including the generation of aluminum oxide layers to promote hydroxyapatite formation. Irradiation-driven compositional variation such as one element over another at the surface differing from the sub-surface can be tuned to specific concentrations.

In an aspect, provided is a biodegradable sponge comprising: a magnesium alloy comprising magnesium having an amount selected from the range of 96% to 97.3%, aluminum having an amount selected from the range of 3% to 3.5% and zinc having an amount selected from the range of 0.8% to 1.2%; wherein the biodegradable sponge is characterized by a porosity selected over the range of 50% to 87%; and having Young's modulus selected from the range of 8 GPa to 29 GPa.

In an aspect, provided is a biodegradable sponge comprising: a magnesium alloy comprising magnesium having an amount selected from the range of 96% to 97.3%, aluminum having an amount selected from the range of 3% to 3.5% and zinc having an amount selected from the range of 0.8% to 1.2%; wherein the biodegradable sponge is characterized by a porosity selected over the range of 50% to 87%; and having Young's modulus selected from the range of 8 GPa to 29 GPa; the sponge having an outer exposed surface and an internal exposed surface provided by a plurality pores; wherein at least a portion of the exposed surface has a plurality of nanoscale domains providing a selected multifunctional bioactivity; wherein the nanoscale domains are generated by exposing the surface to one or more directed energetic particle beam characterized by one or more beam properties.

In embodiments, for example the magnesium alloy further comprises one or more additional components selected from the group of potassium, calcium and maganese. In embodiments, the one or more additional components independently have an amount selected from the range of from the range of 0.01% to 0.05%.

In an embodiment, the selected multifunctional bioactivity is with respect to an in vivo or in vitro activity relative to an unmodified magnesium containing sponge surface, for example, a surface not having the plurality of nanoscale domains. In embodiments, the biodegradable sponge comprises a mesoporous or microporous sponge. In an embodiment, the in vivo or in vitro activity is a change in rate of bioresorption, for example, a decrease greater than or equal to a factor of 2, 5, or optionally 10. In embodiments, the nanoscale domains comprise an increase or decrease in the aluminum content of the domain by greater than or equal to 10%.

In an embodiment, the in vivo or in vitro activity is a decrease in hydrogen generation, for example, a decrease greater than or equal to 5%, 10%, or optionally, 20%. In embodiments, the in vivo or in vitro activity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these. In embodiments, aid enhancement of in vivo or in vitro activity is equal to or greater than 50%, 100%, or optionally, 200%.

In embodiments, the nanoscale domains said nanoscale domains have an increased concentration of Aluminum, for example, to promote the formation of calcium phosphate when exposed to a fluid.

In embodiments, the nanoscale domains comprise an increase in Al₂O₃ content relative to the Al₂O₃ content of other regions of an unmodified magnesium containing substrate surface, for example, a surface without nanoscale domains. In an embodiment, the nanoscale domains are provided between and within pores of the substrate. In embodiments, for example, the nanoscale domains are provided between and within pores of the sponge to a depth of 540 μm from the external surface.

In embodiments, the nanoscale domains characterized by a chemical composition different from the bulk phase of the magnesium containing substrate. In embodiments, the nanoscale domains provide an enhancement in vivo or in vitro activity with respect to cell adhesion proliferation activity and migration greater than or equal to 50%, 100%, or optionally, 200%. In an embodiment, the nanoscale domains provide an enhancement in vivo or in vitro activity with respect to anti-bacterial activity and bactericidal activity greater than or equal to 50%, 100%, or optionally, 200%.

In embodiments, the nanoscale domains provide a local in vivo increase in pH, wherein the pH is increased by 0.5 or more, 1.0 or more, or optionally, 1.5 or more. In an embodiment, the nanoscale domains provide an enhancement of a selected physical property of the substrate, for example, hydrophilicity, hydrophobicity, surface free energy, surface charge density or any combination of these. In embodiments, aid enhancement of selected physical property is equal to or greater than 10%, 25%, or optionally, 50%. In an embodiment, the biodegradable sponge is biocompatible.

The described magnesium sponge may be modified using Direct Irradiation Synthesis (DIS), Direct Plasma Nanosynthesis (DPNS), Direct Seeded Plasma Nanosynthesis (DSDPNS), Direct Soft Plasma Nanosynthesis (DSPNS) or other methods of precise surface modification. In an embodiment, the directed energetic particle beam is a broad beam, focused beam, asymmetric beam, reactive beam or any combination of these. In an embodiment, for example, the one or more beam properties is intensity, fluence, energy, flux, incident angle, ion composition, neutral composition, ion to neutral ratio or any combinations thereof. In embodiments, the nanoscale domains are a surface geometry selected selected from the group consisting of topology, topography, morphology, texture or any combination of these.

In an embodiment for certain applications, the step of directing the directed energetic particle beam onto the substrate surface is achieved using a method other than directed irradiation synthesis (DIS). For example, the invention, includes methods of fabricating a bioactive magnesium containing substrate wherein directed plasma nanosynthesis (DPNS), direct seeded plasma nanosynthesis (DSDPNS) or any combination of these techniques is used to carry out the step of directing the directed energetic particle beam onto the substrate surface to generate a plurality of nanoscale domains characterized by a surface geometry providing a selected multifunctional bioactivity. Accordingly, one of skill in the art will readily understand that certain applications and materials of the invention are achieved using methods that do not include processing via directed irradiation synthesis (DIS).

In embodiments, each of the nanoscale domains are characterized by a vertical spatial dimension of less than or equal to 50 nm. In embodiments, each of the nanoscale domains are characterized by a vertical spatial dimension selected over the range of 10 nm to 250 nm.

In embodiments, the nanoscale domains comprise nanowalls, nanorods, nanoplates, nanoripples or any combination thereof having lateral spatial dimensions selected over the range of 10 to 1000 nm and vertical spatial dimensions of less than or equal to 250 nm. In embodiments, the nanowalls, nanorods, nanoplates or nanoripples are separated from one another by a distance of less than or equal to 100 nm. In an embodiment, for example, the nanoscale domains comprise discrete crystallographic domains.

In an embodiment, the biodegradable sponge is generated by infiltration casting and salt fluxing. In embodiments, the biodegradable sponge has a tensile strength selected from the range of 5 MPa to 20 MPa, for example, in the plateau zone.

In an aspect, provided is a method of fabricating a biodegradable magnesium sponge comprising: i) providing a magnesium containing sponge having a plurality of pores each having an surface; and ii) directing a directed energetic particle beam onto the surfaced, thereby generating a plurality of nanoscale domains on the surfaces; wherein the directed energetic particle beam has one or more beam properties selected to generate the plurality of nanoscale domains providing a selected multifunctional bioactivity.

In an embodiment, the directed energetic particle beam is a broad beam, focused beam asymmetric beam or any combination of these. In embodiments, the step of directing the directed energetic particle beam onto the substrate surface comprises directed plasma nanosynthesis (DPNS), Direct Seeded Plasma Nanosynthesis (DSDPNS), Direct Soft Plasma Nanosynthesis (DSPNS) or any combination of these. In embodiments, for example, the one or more beam properties is intensity, fluence, energy, flux, incident angle, ion composition, neutral composition ion to neutral ratio or any combinations thereof.

In an embodiment, the directed energetic particle beam comprises one or more ions, neutrals or combinations thereof, for example, Ne ions, Kr ions, Ar ions, Xe ions, N ions or a combination thereof. In an embodiment, the directed energetic particle beam is generated from an energetic 02 precursor.

In embodiments, the one or more beam properties comprise incident angle and the incident angle is selected from the range of 0° to 80°. In embodiments, the one or more beam properties comprise fluence and the fluence is selected from the range of 1×10¹⁶ cm⁻² to 1×10¹⁹ cm⁻² or optionally 1×10¹⁶ cm⁻² to 1×10²⁰ cm⁻². In embodiments, the one or more beam properties comprise energy and the energy is selected from the range of 0.05 keV to 10 keV, 0.1 keV to 10 keV, 10 keV to 100 keV, or optionally, 10 keV to 500 keV. In an embodiment, for example, the multifunctional bioactivity comprises bioresorption.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a photograph of a sample of open cell Mg-based foam.

FIG. 2 shows SEM micrographs of the AZ31 sponge sample.

FIG. 3 provides SEM and corresponding EDS of the a) Non treated foam, b) Foam irradiated with 0.4 eV and c) Foam irradiated with 1.2 KeV.

FIG. 4 provides SEM and corresponding EDS of the a) Non treated foam, b) Foam irradiated with 0.4 eV and c) Foam irradiated with 1.2 KeV.

FIG. 5 Chart describing recorded pH over time of various samples.

FIG. 6 Viability test of the a) Non treated foam, b) Foam irradiated with 0.4 eV and c) Foam irradiated with 1.2 KeV.

FIG. 7. illustrates the decrease of corrosion rate between an non-surface modified magnesium-based sponge and two modified samples.

FIG. 8. Testing of modified magnesium sponges. Left panels are cell viability tests where “red” are cells that are dead and “green” stained to show cell viability. Top-center and shows that DPNS-treated samples over the negative control. Lower-center panels show EDS data of composition.

FIG. 9 Stages of the fracture healing process

FIG. 10 Schematic representation of the bone remodeling model

FIG. 11 Bone porosity changes in different axial load conditions

FIG. 12 Bone porosity changes in different transversal load conditions

FIG. 13 Bone porosity changes with three different pins

FIG. 14 Mg balance in human body

FIG. 15 The concept of skeletal tissue regeneration via porous implant

FIG. 16 Replication process

FIG. 17 Porous metal by spray forming

FIG. 18 Configuration of porous Mg foams prepared by different techniques

FIG. 19 Considerations for selection of the alloying elements in a biodegradable Mg alloy

FIG. 20 Mechanical properties of Mg alloys and human bone

FIG. 21 Strategy for materials selection

FIG. 22 Mg foam selected porosity parameters

FIG. 23 EMg assays conditions

FIG. 24 Roughness parameters

FIG. 25 Surface roughness calculation

FIG. 26 AZ31 foams fabricated with different infiltration parameters

FIG. 27 Stereo-micrograph of a) EMg2 and b) EMg6

FIG. 28 2D slice and binary 2D slice from a X-ray μCT, EMg6

FIG. 29 Solid reconstruction using MIMICS

FIG. 30 SEM Image of a) EMg2 and b) EMg6 foams porous surfaces

FIG. 31 SEM Image of EMg6 foam

FIG. 32 SEM Image of EMg2 foam

FIG. 33 Metal concentration levels EMg2 and EMg6 compared with AZ31 composition

FIG. 34 XRD patterns of original ingot

FIG. 35 XRD patterns of four EMg2 samples

FIG. 36 XRD patterns of four EMg6 samples

FIG. 37 Compression test stress-strain curves

FIG. 38 Roughness effect on osteoblast morphology

FIG. 39 A schematic illustration of compression curve of porous metal scaffolds

FIG. 40 Surface properties

FIG. 41 RMS roughness value plotted against ion energy. Values based on

FIG. 42 Distribution of the corrosion test samples in the plate

FIG. 43 Distribution of the liquid extracts of material in the plate

FIG. 44 Distribution of the irradiated samples in the plate

FIG. 45 Main effects plots of energy (E) factor for Weight Loss and pH

FIG. 46 Interaction plots for Weight Loss and pH

FIG. 47 Corrosion products of EMg6 after 96 h of exposition

FIG. 48 400 eV sample corrosion products

FIG. 49 Detailed view of the corrosion products

FIG. 50 Contour plot of Weight Loss vs. Energy. Exposition

FIG. 51 Corrosion rate for each sample

FIG. 52 Viability test results

FIG. 53 SEM image of representative regions populated by cells for each sample type

FIGS. 54A-54C. Surface-cells interactions.

FIG. 55. X-ray μCT a) slide of Mg-based foam and b) binary image of same slide.

FIG. 56. shows infiltration results for three different magnesium foam samples.

FIG. 57. SEM micrograph of EMg1 revealing two types of pores.

FIG. 58. SEM micrograph of EMg2 showing micro and macropores.

FIG. 59. Metal concentration levels of EMg2 and EMg1 compared with AZ31 composition.

FIG. 60. XRD patterns of original ingot featuring peaks from Mg and MgO.

FIG. 61. XRD patterns of four EMg2 samples which show phase similar to original ingot.

FIG. 62. XRD patterns of four EMg1 samples.

FIG. 63. provides compression test stress-strain curves comparing samples EMg1 and EMg2.

FIG. 64. provides a strength versus time comparison between permanent implants, degradable implants and bone.

FIG. 65 provides XRD data of a magnesium alloy treated with DPNS at different fluences.

FIG. 66 provides XRD data of a magnesium alloy treated with DPNS at different fluences.

FIG. 67 provides concentration data of a magnesium alloy treated with DPNS based on the data in FIGS. 65 and 66.

FIG. 68. provides an SEM image of a magnesium sponge treated under 400 eV, Ar+, 10¹⁸ cgs fluence.

FIG. 69. provides an SEM image of a magnesium sponge treated under 600 eV, Ar+, 10¹⁸ cgs fluence.

FIG. 70. provides an SEM image of a magnesium sponge.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Nanoscale domains,” as used herein, refers to features characterized by one or more structural, composition and/or phase properties having relatively small dimensions generated on the surface of a substrate. Nanoscale domains may refer to relief features and/or recessed features such as trenches, nanowalls, nanocones, nanoplates, nanocolumns, nanoripples, nanopillars, nanorods, nanowires, nanotubes and/or quantum dots. Nanoscale domains may refer to discrete crystalline domains, compositional domains, distributions of defects, and/or changes in bond hybridization. Nanoscale domains include self-assembled nanostructures. In embodiments, for example, nanoscale domains refer to surface depths or structures generated on a surface having dimensions of less than 1 μm, less than or 500 nm, less than 100 nanometers, or in some embodiments, less than 50 nm. In an embodiment, nanoscale domains refer to a domain in a thermally stable metastate.

“Nanoscale domain” refers to a surface-modified area positioned on the surface of a substrate. Nanoscale domains may have a periodic or a semi-periodic spatial distribution. For example, nanoscale domains include topology, topography, spatial distribution of compositions, spatial distribution of phases, spatial distribution of crystallographic orientations and/or spatial distribution of defects. Nanoscale domains of some aspects are useful for providing a selected multifunctional bioactivity, a selected physical property or a combination thereof.

“Selected multifunctional bioactivity” refers to an enhancement of in vivo or in vitro activity with respect to a plurality of biological or physical processes. In embodiments, for example, multifunctional bioactivity is enhanced relative to magnesium based substrate surface not having said plurality of nanoscale domains. In an embodiment, for example, a selected multifunctional bioactivity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseoconductive activity, osseointegration activity, biocorrosion activity, cell differentiation activity, immuno-modulating activity during acute or chronic inflammation or any combination of these. In an embodiment, for example, a selected multifunctional bioactivity is a modulation in the immune response to a foreign body (e.g. the implant). In an embodiment, for example, a selected multifunctional bioactivity is an enhancement or inhibition of one or more protein interactions.

“Directed energetic particle beam,” as used herein, refers to a stream of accelerated particles. In embodiments, the directed energetic particle beam is generated from low-energy plasma. In some embodiments, directed energetic particle beam is a focused or broad ion beams capable of delivering a controlled number of ions to a precise point or area upon a substrate over a specified time. Directed energetic particle beam may include ions and additional non-ionic particles including subatomic particles or neutral atoms or molecules. In embodiments, directed energetic particle beams provide individual ions to the target location. Examples of directed energetic particle beams include focused ion beams, broad ion beams, thermal beams, plasma generated beams and optical beams.

“Beam property” or “beam parameter” refer to a user or computer controlled property of beam, for example, an ion beam. Beam parameter may refer to incident angle with a target substrate, fluence, energy, flux, beam composition and ion species. Beam parameters may be adjusted to provide selected interactions between the beam and the target substrate to generate specific nanostructures or enhance specific properties of the substrate including rate of bioresorption. Beam parameters may be controlled by a variety of means, including adjustments to electromagnetic devices in communication with the beam, adjusting the gas or energy source used to generate the beam or physical positioning of the beam in reference to the target.

“Vertical spatial dimension” refers to a measure of the physical dimensions of a nanoscale domain perpendicular or substantially perpendicular to the planar or contoured surface of a substrate. In embodiments, vertical spatial dimension refers to a height or depth of a nanoscale domain or the mean depth of a surface modification, for example, a crystalline or compositional domain.

“Lateral spatial dimension” refers to a measure of the physical dimensions of a nanoscale domain parallel or substantially parallel to the planar or contoured surface of a substrate.

“Magnesium or Magnesium alloy substrate” refers to any substrate composed of magnesium including specific magnesium alloys described herein. In some embodiments, magnesium alloy may refer to alloys containing magnesium but in which magnesium is not the primary component. In other embodiments, magnesium alloy refers to alloys in which magnesium represents more than 25%, or optionally 50%, of the alloy. Magnesium and magnesium alloys may include an oxide layer, for example magnesium oxide or aluminum oxide, including on the surface being modified.

“Porosity” or “porous magnesium” refers to substrates or magnesium surfaces having individual or networked voids at or near the surface of the substrate. Porosity may be nanoscale, microscale or larger. As described herein, substrates may have porosity prior to any plasma treatment (e.g. porosity formed during substrate formation such as sintering). In some embodiments, pores may be formed, enlarged or altered by the treatment of directed plasma, including forming nanopatterns on interior pore surfaces or walls between individual pores. Porous magnesium includes magnesium-based sponges and foams.

“Multiplexing” refers to simultaneously modifying the target substrate in more than one way, for example, by providing two or more directed particle beams at the substrate having different properties, for example, to generate or modify at least one nanoscale domain (e.g. nanoscale features, crystalline domains, compositional domains, distributions of defects, changes in bond hybridization. In some embodiments, for example, a single directed particle beam may have one or more beam properties to generate or modify multiple nanoscale domains on the substrate. In embodiments, multiple direction particle beams are generated from the same plasma source.

The technology as described in the present disclosure includes an advanced nanomanufacturing process as described herein, advanced tools particular for this process and a number of unique nano-scale structures generated as a result of the processing.

In one embodiment, provided is an atomic-scale additive nanomanufacturing process capable of transforming materials with multi-functional properties without the need for expensive heat cycles, toxic chemical processes or thermodynamic limitations of material compatibility in processing. The interface between plasma and material becomes an open thermodynamic system driven far from equilibrium by a rich variety of physical mechanisms, including high-energy kinetic disordering, compositional phase dynamics, and the emergence of metastable material states. The instabilities that arise due to these mechanisms lead to the evolution of well-ordered nanostructures, the compositional and morphological characteristics of which dictate the material properties.

“Directed energetic particle beams” are drawn from a low-temperature plasma (gas discharge) in a manner that controls the energy, species and intensity of the respective beams from the aforementioned plasma. This technique may be called directed plasma nanosynthesis (DPNS) herein. The particles may be combined with additional reactive atoms and/or surfactants that interact with material surface inducing variation in a number of properties including: surface chemistry, composition, topography, topology, charge density and bond hybridization. In some cases the technology can manipulate these properties independently providing for multi-functionality on the material surface without modification to the bulk material. Depending on material type the energetic particles are selected both in mass and species to result in the desired material property (e.g. hydrophobicity, anti-bacterial for biomaterials, etc. . . . ). The material can be a polymer, metal, ceramic, or semiconductor and the synthesis can be done over large areas, at room temperature and over a short period of time (e.g. seconds). DPNS is designed to independently modify surface topography, composition and charge density yielding increase of surface energy and surface-to-volume ratios by factors of 50-100% and 100-1000, respectively. DPNS include a use of a plasma source enabling the modification of existing product materials (e.g. on a biomedical stent, implant device, etc. . . . ) improving their properties or synthesizing completely new class of materials. DPNS enables a single source that addresses the problematic use of thin-film coatings for bioactive interfaces, which can potentially lead to osteolysis and chronic inflammation. Coating disintegration and delamination is also a prevalent problem that cannot be solved with current synthesis approaches that include: electrophoretic deposition, anodization, electrolysis, reactive DC magnetron sputtering, RF plasma sputtering, and x-ray sintering among others. One of the primary issues with these conventional technologies is the formation of the interface between the coating and biomedical material substrate. Therefore, features of DPNS are: 1) low cost (e.g. they are a low-temperature process; heat cycles during synthesis make-up 30-40% of the current processing cost of surface modification techniques), 2) green and sustainable (does not require harsh chemicals for synthesis and can enhance usability of natural materials), and 3) scalable (particle irradiation can be conducted throughput levels of about 1012 micron2/hr or a modification of a 6-inch wafer in about 10 seconds). Another added benefit and potentially disruptive approach is the ability to modify a surface composition and chemistry independent of the topography with high-fidelity. In other words, inducing a surface that can potentially enhance cell adherence and proliferation while repelling bacteria, for example.

“Directed energetic particle beams” include DPNS to produce nanostructures on the substrate surface. In first step, a substrate is provided in a fixture, not shown, where the directed energetic particle beam from a low temperature plasma may operate on the substrate with a surface. The directed energetic particle beam(s) from a low temperature plasma source are directed to the substrate surface in accordance with parameters and/or properties that correspond to a desired nanostructure topology. The parameter control may occur in an automated fashion, such as under the control of a numerical control device or special purpose computer, including a processing device and a memory containing programming instructions (not shown). In an optional step, additional beam(s) may be generated and directed to the surface of the substrate also in accordance with parameters and/or properties that correspond to a desired nanostructure topology. Optional step includes depositing one or more agents on the surface of the substrate.

“Directed energetic particle beams” can be derived from plasma processing sources known in the art, for example, Tectra GmbH Physikalische Instrumente (GENII PLASMA ION SOURCE) and Oxford Instruments (ISE 5 ion sputtering source). Also SVT Associates, Inc. provides the RF-6.02 Plasma Source. While the principles and methods for creating plasma sources are known, these plasma processing methods create only mono-directional particle beams, which limits their usage to flat, 2D surfaces. Methods for performing DPNS as 3D are described in, for example, U.S. Patent Application Ser. No. 62/483,105, “Directed Plasma Nanosynthesis (DPNS) Methods, Uses and Systems,” filed Apr. 7, 2017, the disclosure of which is incorporated by reference herein in its entirety.

“Directed energetic particle beams” include low temperature plasmas and gasiform plasmas with electron temperature under 10 eV, electron density typically from 1014 to 1024 m⁻³. In general, low temperature plasmas have a low degree of ionization at low densities. This means the number of ions and electrons is much lower than the number of neutral particles (molecules). Different particles inside the plasma, i.e. neutrals, ions and electrons, can have different temperatures or energies. Indeed, in many applications, the background gas is near room temperature. In this regard, gas phase reaction activation energy can be driven by electron impact rather than thermally and the substrate is not subjected to extreme heating, which is useful for functionalizing temperature sensitive substrates such as polymers. For “directed energetic particle beams” one or more beam properties is the gas, intensity, fluence, energy, flux, incident angle, species mass, charge, cluster size, molecule or any combinations thereof. In an embodiment, for example, the directed energetic particle beam comprises one or more ions, neutrals or combinations thereof. In embodiments, the one or more beam properties are the ion composition, neutral composition, the ratio of ion abundance to neutral abundance or any combination of these. In embodiments, the directed energetic particle beam is incident upon the substrate from a plurality of directions.

Using “directed energetic particle beams” nanostructures may be obtained as function of energetic particle species, fluence and incident angle with respect to the surface normal. For example, energetic particle species may include those obtained from gases such as Kr, Ar, Ne, Xe, H, He, 02 and/or N2. Fluence can be, for example, between 1×10¹⁷ to 1×10¹⁸ particles per second per square meter, but may vary from 0.1×10¹⁷ to 50×10¹⁷. In some embodiments, fluence is 1×10¹⁷, 2.5×10¹⁷, 5×10¹⁷, or 1×10¹⁸ particles per second per square meter. Finally, incident angle may be varied in single degrees between the angles of 0 and 80 degrees, in some embodiments, for example, 30 degrees, 60 degrees, and 80 degrees. In some embodiments, for example, the plasma-based source of the invention provides one or more directed particle beams having a distribution of incident angles, such as a distribution of incident angles characterized between 0 and 90 degrees with respect to the sample surface normal.

The invention may be further understood by reference to the following non-limiting Examples that expand on certain aspects and embodiments of the invention.

### Example 1—Nanostructured Bioresorbable Magnesium-Based Sponge

This example describes bioresorbable and bioactive metallic sponge material for bone tissue engineering. This material has two overall functions: 1) Bulk properties: bone-like structure material that resorbs in the body and 2) Surface properties: bioactive surface with enhanced osseointegration and osseoconductive properties. The surface properties are designed in conjunction with the bulk properties of the metallic sponge material.

A metal sponge is a structure which consists on spaces filled by an interconnected metal coexisting with an interconnected network of empty space. The specific properties of this kind of structures are low stiffness and low density while maintaining mechanical properties. The sponge architecture involves variables that can be targeted by DPNS such as interconnected porosity, ranges of pore size, volume faction, surface area, pore shape and pore distribution.

Bulk properties are obtained by infiltration casting without a controlled atmosphere using a combination of salt fluxes. The obtained foam simulates the structure and porosity of natural human bone serving as a guide for new bone formation across the bony defect site while it is resorbed by the body. This advanced biomaterial also by design of the sponge

Surface properties are obtained by low-energy directed plasma nanosynthesis (DPNS), that when combined with the interaction of human body fluid environment triggers specific bioactive functions. The synergistic interaction between irradiation parameters and immersion medium influences the structure of the surface and consequently the biomolecular attachment response as well as provides cues/instructions to osteoprogenitor cells. As a result, the materials may be tailored to interface to a patient's specific chemistry near the region for osseointegration and osseoconduction by specific DPNS parameters that result in favorable Ca/P phases for bone development and growth. In addition, the DPNS modification also tunes the resorbability of the material to control the recovery length of time simultaneously with bone tissue growth. In addition, the inherent nanostructuring of the surface results in a bactericidal interface effective for anti-bacterial functionality

The bioactived metallic sponge has provides osseoconduction and osseointegration for prosthetic and orthopedic platforms. Additionally, the material may provide one or more of the following properties:

1. A resorbable material that can be designed to be absorbed in the body for a predetermined period of time, enabling the control of resorbability.
2. Bio-mechanical strength comparable to that of bone using a magnesium-based sponge (e.g. cellular metal) structure,
3. High-fidelity control of hydrogen production from released from the magnesium-based material during the process of bioresorption
4. Enhanced cell proliferation for faster bone integration by means of surface nanostructuring,
5. Intrinsic anti-bacterial properties by surface morphology
6. Intrinsic growth of hydroxyapatite mineralized interface (HA) tailored to the specific chemistry of the body when exposed in-vivo tuned by the specific DPNS parameter configuration. HA is intrinsically accelerated by the irradiation-driven mechanism bringing aluminum atoms in the Mg alloy to the surface enhancing the speed of HA growth.

### BACKGROUND

Most bone defects are commonly treated with autologous bone transplantation. But these grafts have limitations due to the high rates of rejection and insufficient source for extractions (cadaveric donors). Since the available bone grafts is limited in quality and quantity, a variety of approaches have been developed to reconstruct bone defects. Some hard metals such as Ti and its alloys, stainless steels, Co-based alloys, Ni—Ti, Ag, Ta, and Zr have been applied in load-bearing requests and hard tissue implants, due to their high strength, ductility and corrosion resistance. Nevertheless, these metals cannot degrade spontaneously, and a second surgical procedure is usually needed to remove the implants from the body after the tissues have completely healed. Repeated surgeries increase both cost and patient morbidity. Also, after the healing stage, the presence of a permanent implant usually results in a number of adverse effects, such as osteoporosis and stress shielding due to a mismatch in elastic modulus between the bioinert material and bone. The ability to provide for a multi-functional biomaterial that not only was biocompatible but also provide the proper biomechanical properties together with bioactive properties to enhance osseoinduction and osseointegration has been one of the greatest challenges to current technology in orthopedic implant existing art. Designing materials to integrate with bone has been a significant technological gap. The complex biological processes tied to both the immunology conditions and osteoblast differentiation and proliferation remain a challenge in the design of the ideal bone replacement synthetic materials. Prior art has focused on the use of HA (hydroxyapatite) coatings that are known to integrate with bone given their Ca and P content mimicking bone's physical, chemical and biological properties. However, HA coatings are known to have extensive issues involving delamination and poor osseoinduction while enhancing osseoconductive processes that are unfortunately non-sustainable and ultimately lead to failure of the biomedical implant.

Magnesium based biofoams with both porous structure similar to the architecture of natural bones and suitable mechanical properties and biointeractive properties are described herein. In some embodiments, provided is a scaffold material that undergoes a complete biodegradation process after a proper healing time period. Magnesium (Mg) and its alloys are materials that have changed the paradigm of permanent biomedical implant fixations. Nevertheless, the degradation rate is one of the biggest complications for a practical implementation of Mg as biomaterial. Mg is a highly reactive material and has a lower corrosion resistance in chloride-containing environments. This condition limits its physiological application, and restricts its extensive use in orthopedic applications. Therefore, it's advantageous to devise a method to improve magnesium's corrosion resistance and bioactivity, while maintaining the lightweight properties of Mg alloy sponges.

Since bioactivity and degradation resistance of a bone substitute are factors for repairing and healing fractured bones, we have invented a technique using directed plasma nanosynthesis (DPNS) extracting low energy ion irradiation as a suitable method for surface modification of Mg-based foams. This modification enables high-fidelity selectivity of enhanced anti-corrosion properties and cellular responses without affecting the required bulk attributes of an Mg-based foam for bone integration. Additionally, provided is a bioactive Mg-based metal sponge material that adapts to the biological environment of the body. In particular, our bioactive interface is designed by the combination of the intrinsic microstructure and composition of the bulk Mg alloy sponge and the irradiated interface that renders this specific surface bioactive property or properties.

When fabricated with DPNS, the bioactive interface grows an HA interface when exposed to the specific chemical environment of the body. DPNS parameter exposure may tailor a biointerface to adapt and respond to the biological environment intrinsic on its surface. Therefore, there is no need for the use of a bioactive thin-film coating that can delaminate or the need for highly toxic surface modification as currently common in the art.

### Surface Modified Mg-Based Sponge

Described herein is a magnesium-based sponge for medical uses which is highly porous and biodegradable. This material is characterized by the formation of a targeted surface induced by low energy irradiation. Further, the material degrades in a body and cells simultaneously grow into it. Therefore, excellent interfacial strength is provided between implant and tissue. Also, the material can be appropriately used as a bone substitute or for bone treatment, and used as orthopedic, dental and plastic surgical material.

An exemplary magnesium metal alloy is AZ31 Mg-based alloy. Foams are manufactured using a preform of salt particles. NaCl particles are dried a low temperature and deposited into a mold. Then, the metal pieces were placed over the salt template. Mg is protected from oxidation by the use of fluxes, therefore, no protective gas atmosphere is needed. The temperature is slowly increased from room temperature to around 700° C. Finally, vacuum pressure is applied to force the metal infiltration into salt spaces and the salt preform is dissolved in water to reveal the interconnected network of pores.

The magnesium casting processes commonly require the use of controlled environment with shielding gas to prevent interaction with oxygen. In this case, instead of using mixed gasses such as CO₂ and SF6, a flux has been used with the same function. This formulation of salts may build a protective layer on top of the melt and settle out after this layer has been disturbed.

Samples are exposed to a low-energy Ar+ irradiation with different ion energy values. Then, samples are immersed in a solution with an ionic composition similar to that of the human body with a 1.67:1 Ca:P ratio. The resultant surface depends on the modulation of the irradiation parameters and the synergistic interaction with the inorganic salts present in the immersion medium. The foam surface can further be customized with one or more predetermined biofunctionalities.

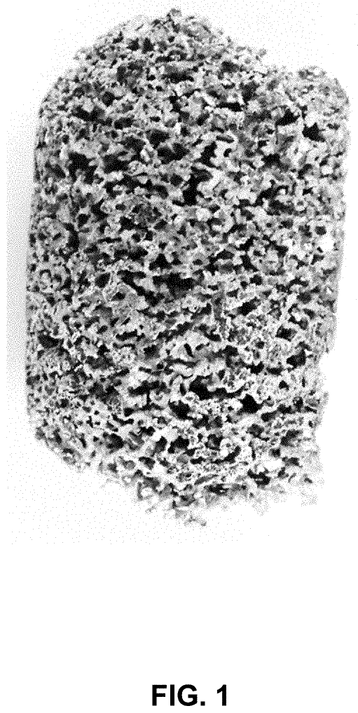

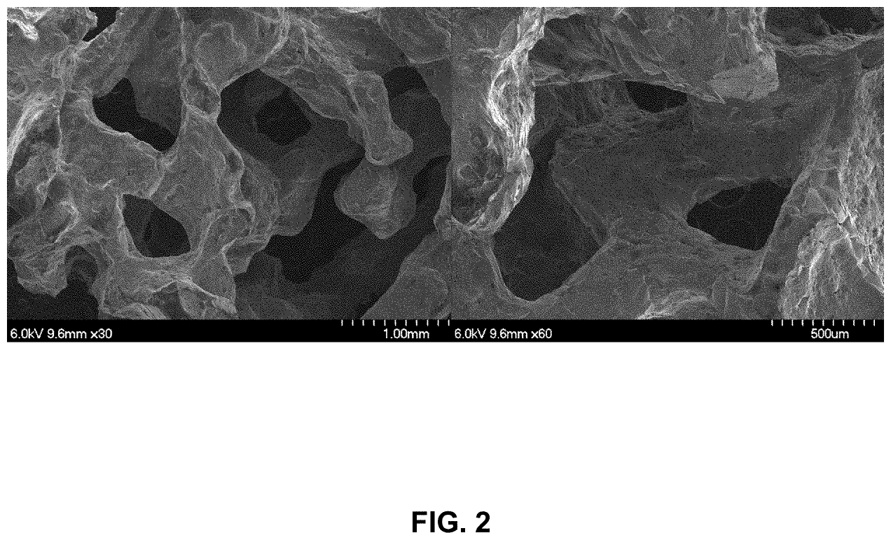

Representative samples of the open-cell Mg-based foam, with different pore sizes, are shown in FIG. 1. FIG. 1 illustrates a sample a homogeneous distribution of pores with irregular form and cell size equivalent to salt particles size. The corresponding SEM micrographs of the sponges are shown in FIG. 2, which demonstrates the open-cell structures with interconnected pores.

An exemplary, improved formulation of salt fluxes is:

### a KCl=0.3219X

### MgO=0.16X

### MgCl2=0.5375X

### CaF2=0.1608X

Where X is the mass of Mg.

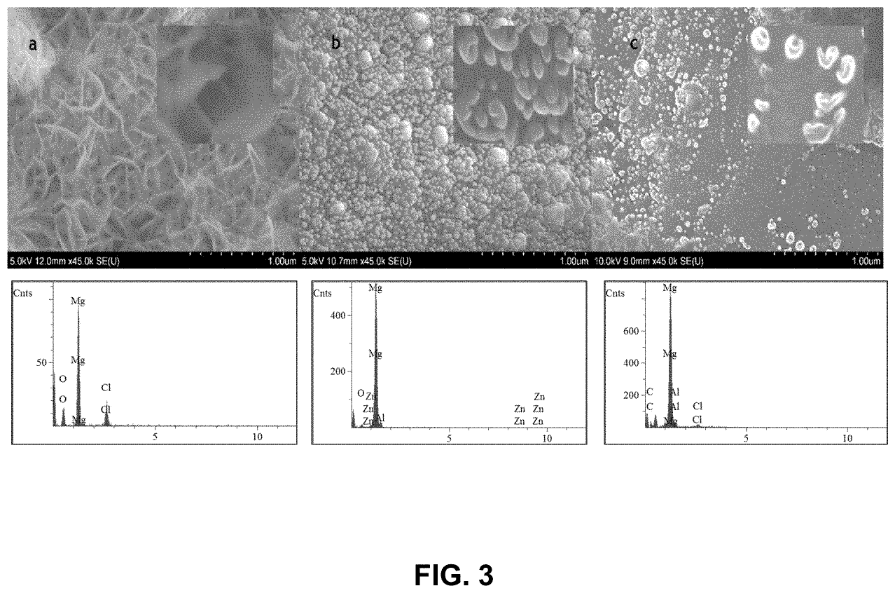

The AZ31 random foams are irradiated with the ion beam directed onto them. The results show that different ion energy values responded to different microstructures and morphologies. The MgO present in the no treated foam is replaced by nanostructures that raised up with 1.2 keV and in a more crowded form in 400 eV case. Also, the surface concentration of aluminum is not detected in the non-irradiated and 1.2 keV irradiated foams, which indicates that the aluminum content in the surface is increased up in the foam irradiated with 400 eV (see FIG. 3).

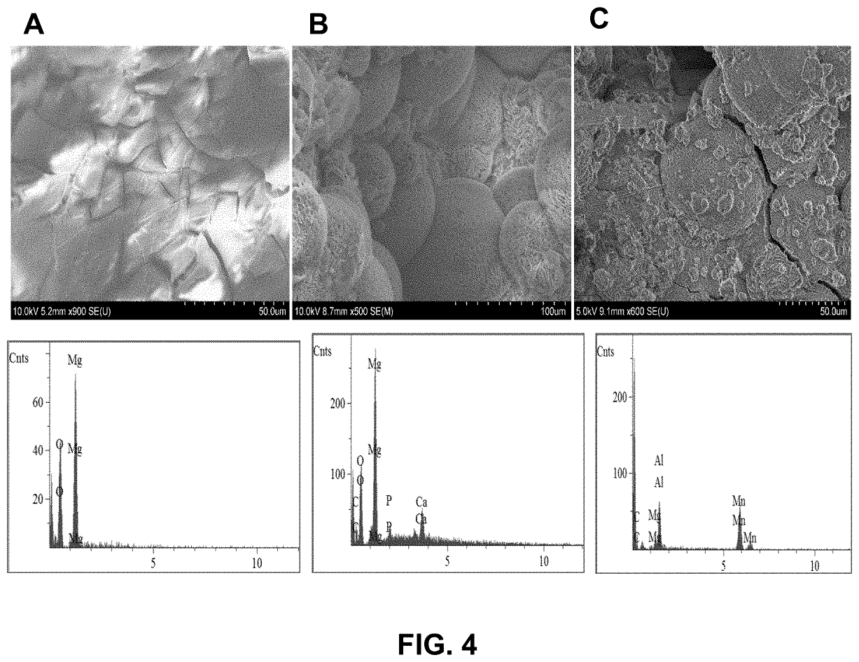

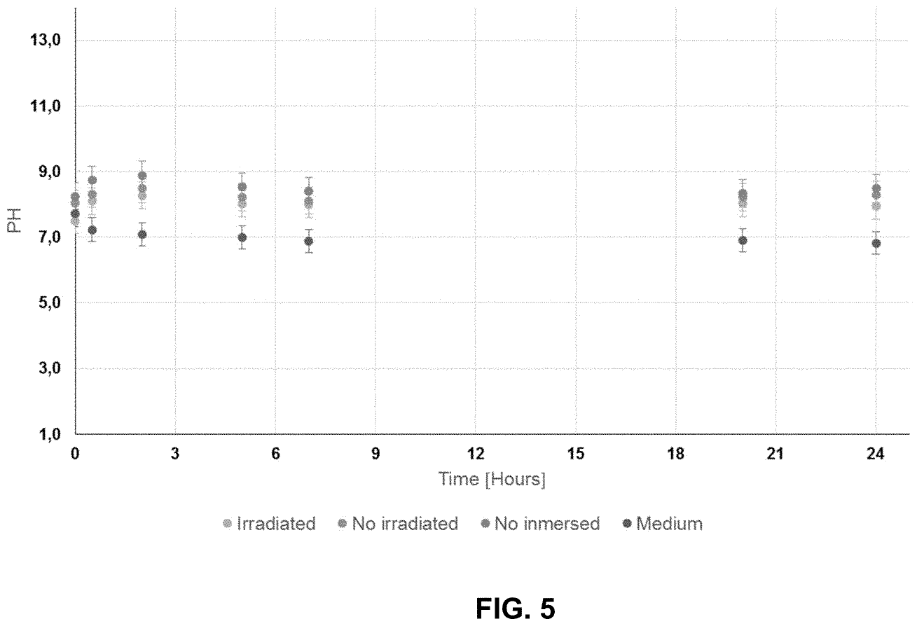

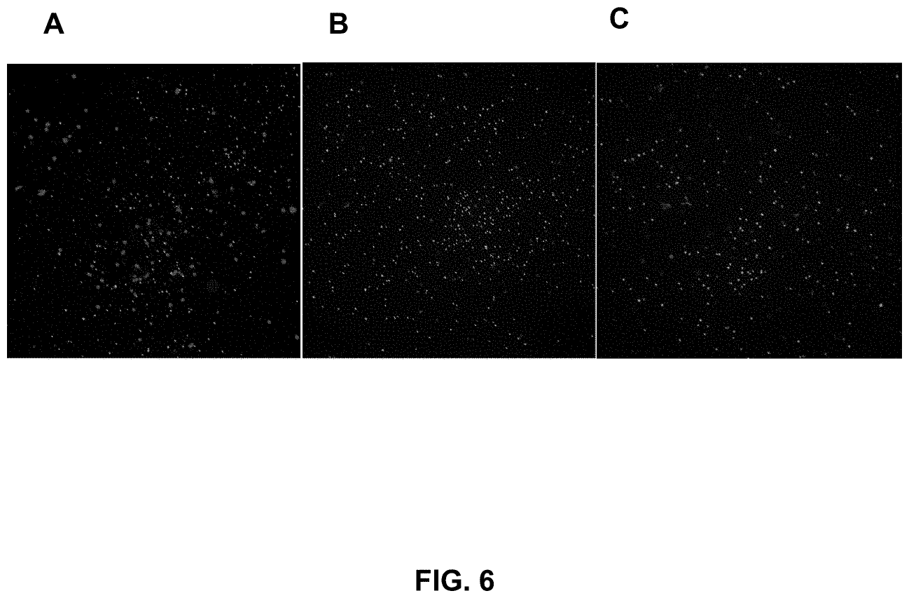

The ion energy value has proved to have an influence on the interaction of the surface with surroundings to which it is exposed. After irradiation, all the samples are immersed in a medium with ionic composition and concentration similar to that of the human environment. The irradiated surfaces were covered by a layer of corrosion products, FIG. 4. In both cases, the formed layers slow the diffusion of water and ions to the surface of the sample thus the pH was successfully lowered, FIG. 5. Also, the biological behavior was significantly improved and cells are more viable and proliferated in the irradiated cases, FIG. 6. The surface irradiated with 400 eV was also in favor of forming CaP phases which are important in the process of bone development and potently increase alkaline phosphatase (ALP) activity and extracellular accumulation of proteins.

In general, the methods used to obtain open porosity metal foams do not control morphological aspects of pores (size, shape, and distribution), which is possible by the process of infiltration of preformed salt.

The use of gasses such as CO2 and SF6 to protect the magnesium alloy during melting processing is not necessary. In this case, fluxes in casting magnesium alloys have the function of protecting the liquid metal from the atmosphere, in order to not react with oxygen. In this case, the flux takes a second role more relevant by creating a contribution from the chemical point of view on the surface of the alloy, which then leads to the desired behavior in surface modification and biocompatibility.

The aim of the surface design is to alter the surface while not changing the bulk properties. Ion energy irradiation modification of the metal sponge is an effective method to diminish the progress of corrosion in biological environments, it is beneficial for the growth of Ca/P coatings on Mg-based alloys, and other targeted complexes, including higher coating coverage and thickness growth. These products formed on the surface are able to impede the flow of electrolyte towards the foams surface while stimulate the cells growth and enhance the osteogenic behavior.

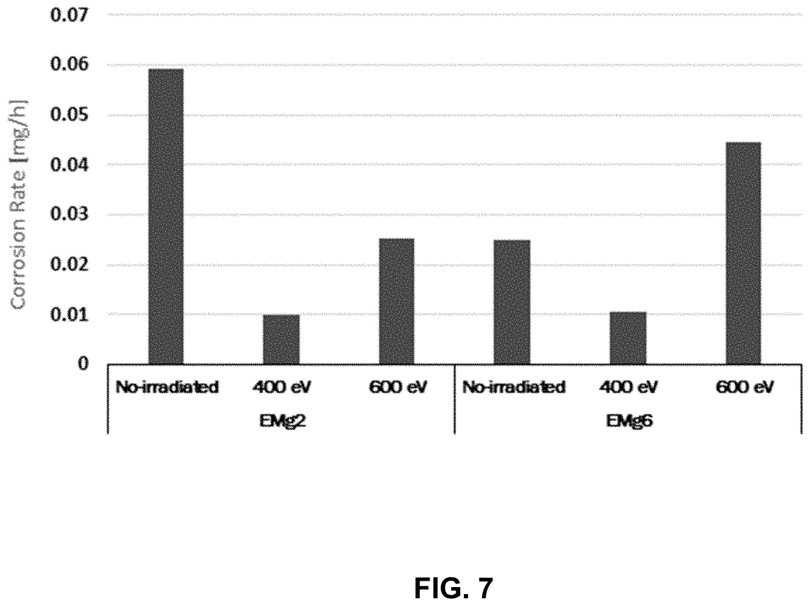

An assay of weight changes after direct immersion in simulated body fluid indicated that complete bioresorption of a porous implant may take up to four months, which may be less in vivo. The bioresorption mechanism depends on the Ca/P phase attached to the surface. A well tuning of the DPNS variables leads to a more crystalline HA, which makes the surface less soluble in the medium, thus, decreasing the fraction of exposed Mg and thus reducing the bioresorption or degradation rate. FIG. 7 illustrates that the corrosion rate is decreased by a factor of 6 times. Sample EMg2 has 1000 micron pores and sample EMg6 has 500 micron pores.

Different DPNS parameter configurations may generate different microstructures and morphologies. The random sponges were irradiated with the ion beam directed onto them. The resultant morphology depends on the location in the spongy structure. Particularly, in the top surface, the MgO and Mg(OH)2 present in the non-irradiated sample is replaced by nanostructures which vary in size, thickness, spacing and quantity depending on the DPNS parameters (400 eV, Ar+, 1E18 fluence cgs). The nanostructures also represented a passivated state of the Mg alloy, rather than a surface that would be comparable with traditional anodization techniques.

The structure of the surface influences molecular attachment. The quantity of alumina on the surface is varied by regulating DPNS parameters and thus, nanofeature morphology. Thus the alumina-rich phase may act as a cathode changing the rate of magnesium dissolution, which directly influences the chemistry of the formed HA or Ca/P ratios on the surface.

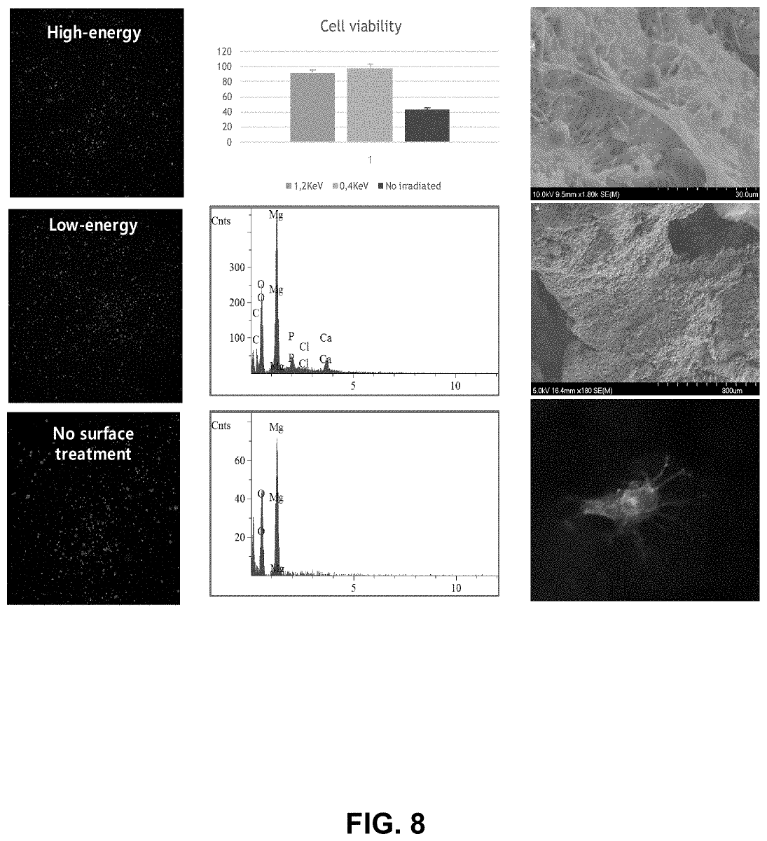

The characteristics of Mg sponge bioactive interfaces are dictated by the parameter space of DPNS used. A specific example is shown in FIG. 8 under prototypical conditions, namely immersing the irradiated and non-irradiated samples in simulated body fluid (SBF), which based on the compositional and morphological change induced by the DPNS method “prepares” the surface in a manner that when exposed to SBF spontaneously generates from the Mg alloy substrate Ca/P phases that can be tuned to form hydroxyapatite (HA) without any coating used. This phase transformation is driven namely by the content of Al segregated by ion-induced mechanisms to the surface of the Mg sponge.

In FIG. 8, the left panels are cell viability tests where “red” are cells that are dead and “green” stained to show cell viability. The data is then plotted in top-center and clearly shows that DPNS-treated samples over the negative control enhanced cell viability by a factor of approximately 100% more viability. In center panels show EDS data of composition demonstrating that irradiated samples indicate a Ca/P phase formed and seems correlated to cell viability. The viability of cells is observed in right panels with respect to a healthy morphology and proliferation over the surface of Mg sponge.

Surface modification also provides additional bioactivity on magnesium sponges. For example, magnesium may act as an antibacterial because it provokes pH increases. This effect may help to reduce implant infections and antibiotic treatment. In addition, bacteria are known to break up on nanostructured surface and in particular protruding nanostructures. With the DPNS-generated nanostructures around the surface, anti-bacterial behavior may be induced.

Example DPNS parameters useful for magnesium sponges and foams are provided in Table 1.

TABLE 1
Example magnesium sponge DPNS parameters.

| | Energy | | Angle | Fluence |
|---|---|---|---|---|
| Sample | (eV) | Gas | (°) | (cm⁻²) |
| Mg sponge | 400 | Ar | 0 | 1.00E+18 |

### Example 2—Development of a Cellular Metal Based on Biodegradable Magnesium Alloys for Bone Fixation Abstract

Biodegradable metals are breaking the paradigm in biomedical applications to consider only corrosion resistant materials. In particular, magnesium is a promising candidate for obtaining temporary implants. However, there are limitations in the real application or Mg due to its rapid degradation rate and poor integration with the surrounding tissue. Hence, in this thesis the development of a cellular metal based on biodegradable Mg alloys, for bone tissue engineering is described.

In order to reach the goal, an Mg-based alloy was selected taking into account the behavior of the reported alloys used in bone tissue applications. Once Mg alloy was selected, the structure of a porous system was defined considering the bone organization. With this data, the selection of the process variables, and the fabrication of the metal foam were carried out. Characterization experiments were performed and it was determinate that a 500 μm pore size foam has a structure closer to bone.

Furthermore, a modification route intended to create a hydrophobic surface by low-energy irradiation was proposed as a method to improve the corrosion performance of the porous material. Two ion energy values were applied. Then, irradiation modified the surface, and a great effect when improving corrosion resistance and cells adhesion to the surface was noted. The obtained foams can be considered and advantage as potential material for bone tissue regeneration from a bone-implant interface point of view.

### Introduction

The conditions musculoskeletal system undergoes are highly complex. There are many diseases and functions related to this biological system. Bone defects, cases of nonunion and compromised fracture healing are an increasing social and medical problem fueled by several risk factors, including the traumatic mechanism, aging of the population, and soft tissue injuries associated with the trauma [1], [2]. The greatest effect of this medical condition is on the limitations for physical activity and bone growing in young patients[3]. TechNavio's analysts have estimated that the world orthopedic manufacturing market will grow by 11.05% of compound annual growth rate (CAGR) from 2012 to 2016 [4].

Most bone defects are commonly treated with autologous bone transplantations [2]. But this grafts have limitations due to the high rejection rates and insufficient sources for extractions (cadaveric donors). Since the available bone grafts are limited in quality and quantity, a variety of approaches have been developed to reconstruct bone defects. Therefore, new artificial strategies are required.

Ti and its alloys [5], stainless steels [6], Co-based alloys, Ni—Ti, Ag, Ta, and Zr [7] have been applied in load-bearing requests and hard tissue implants, due to their high strength, ductility and good corrosion resistance. Nevertheless, these metals cannot degrade spontaneously, and a second surgical procedure is usually needed to remove the implants from the body after the tissues have completely healed. Repeated surgeries increase both costs and patient morbidity [8]. Also, after the healing stage, the presence of a permanent implant usually results in a number of adverse effects, such us osteoporosis and stress shielding due to a mismatch in elastic modulus [7].

The ideal bone substitute material should be porous, osteoconductive, biodegradable, and strong enough to fulfill the required load-bearing functions. Several scaffold materials, including natural polymers [9], synthetic polymers [10], copolymers [11], ceramic scaffolds [12], bioglass [13], composites [12] and metallic scaffolds [14], have been widely studied and applied clinically in hard tissue implants, specifically in load-bearing applications. The results demonstrate that the major limitation of these porous materials is their inadequate mechanical properties. For instance, the weak nature of the ceramics or the very low strength and Young's modulus of the polymers, lower than those of real human bones, have severely limited their applicability.

On the contrary, metals possess higher mechanical properties than human bones, as a result, stress concentrations in the bone will be less than usual and this phenomenon can induce bone resorption. Moreover, these synthetic bone replacements poorly integrate with the cells and surrounding host tissue, resulting in unsatisfactory surgical outcomes due to uncooperative surface environment [15].

It is crucial to develop new biofoams with both porous structure similar to the architecture of natural bones, and suitable mechanical properties. Furthermore, a scaffold material undergoing a complete biodegradation process after a proper healing time period is even more desirable. Magnesium (Mg) is the material that has changed the paradigm of permanent fixations. This element is having greater impact on the scientific community because it combines the property of being both biocompatible and bioresorbable, while is thought to accelerate bone regeneration [16]. In fact, most of Mg in the human body is located in the skeletal tissue, and is an important element for bone growth [17].

Based on these advantages, Mg foams have been already analyzed in the literature on tissue regeneration [16], [18]. However, as a biomedical material, the high corrosion rate of Mg and Mg-based alloys has been observed to result in high concentrations of Mg⁺ and H⁺ gas release. In spite of Young's modulus of pure Mg (45 GPa) [19] is close to that of bone (20 GPa) [20], the yield strength of pure Mg is approximately 20 MPa, lower than that of human long bone (106-133 MPa) [21]. Therefore, it is essential to take relevant measures to control its corrosion rate and to improve its mechanical properties.

The fabrication routes of Mg-based foams have not yet been widely studied and the process parameters necessary to obtain the desired structures are not yet well known. In the early stage of the investigation of porous devices; fabricated with Mg-based alloys, the main objective is to develop manufacturing processes to create sponge-like architectures and understand the resulting mechanical behavior [22]-[27]. Also, the relationship between surface of porous Mg devices and degradation behavior has received limited interest and it is still a controversial issue. These results indicate the need for further research on the surface design, characterization of corrosive behavior, and the surface effects on the possible biological response. Therefore, it is necessary to develop a new structure similar to bone organization and determine the preliminary surface role on corrosion rate and cells behavior.

During the last decade, great interest in porous scaffold substitutes has emerged because of their applicability in bone tissue engineering [28]. Porous material provides the necessary support for cells to grow, proliferate, and preserve their differentiated function. Hence, the shape and the properties of the new tissue are defined by the scaffold geometry [29]. This example provides a review of Mg and its alloys used as biomaterials and a description of their properties and applications.

Bone tissue engineering is the gold target for this work, therefore, the theory of fracture healing process, and the introduction of bone replacements as a strategy in this field of orthopedic surgery is described as well. In that way, stress shielding as a consequence of removal of normal stress by an implant, and the suitability of Mg for its use in biofoams will be discussed taking into account the role of Mg in the human body.

Bone is the second most frequently transplanted tissue [30]. Unlike other tissues, bone can be regenerate and heal itself. However, in intense fractures or large bone defects, the self-repair ability of the bone can fail resulting in slowed unions or non-unions [2]. A bone scaffold is defined as an implanted material that promotes bone healing through osteogenesis, osteoinduction, and osteoconduction. This is a relatively complex device that should be degradable, allow cell attachment and provide mechanical support in order to avoid bone repair failure.

There have been extensive research efforts in biomaterials and tissue engineering dedicated to the production and application of bone scaffolds [28], [31]-[33]. A broad agreement in the biomaterials community determines that vascularization plays a crucial role in tissue engineering [28], [32]. Therefore, a complete explanation about current understanding of bone healing in fractured long bones is provided in this section.

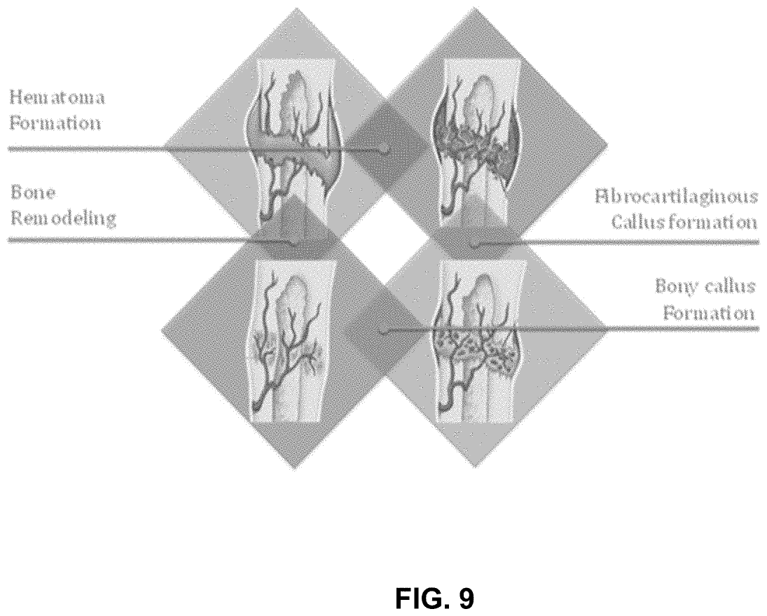

Fracture healing is a proliferative physiological process which can return the bone to its original mechanical conditions. This repairing process occurs in four intersecting stages [34]: I) the reactive stage or hematoma formation; II) the reparative stage or fibrocartilaginous callus formation; and III) the final remodeling stage or bony callus formation; and IV) bone remodeling. Each step of the bone healing process is shown in FIG. 9.

In the early stage, few hours after fracture, the extravascular blood cells form a blood accumulation known as hematoma. Then, inflammatory cells and fibroblasts infiltrate the bone. This permeation results in the formation of an unattached cumulate of cells, interspersed with small blood vessels, identified as granulation tissue. Ingrowth of vascularization and migration of reparative mesenchymal cells occur as well. At that point, reparative stage derives and periosteum cells start replicating (cells proximal to the fracture become chondroblasts and form hyaline cartilage, distal cells develop into osteoblast and form woven bone). At the same time, the fibroblasts within the granulation tissue begin to define a stroma to support the vascularization [34], [36].

Once vascular ingrowth progresses, osteoid is secreted and mineralized. This new tissue grows in size until it meets its homologues from the other parts of the fracture. Then, a soft callus starts forming around the repaired site. The next step is the replacement of the callus with lamellar bone while healing bone is colonized by channels containing microvessels and the bone starts restoring to its original shape and properties. At that moment, in the remodeling stage, trabecular bone is substituted with compact bone. This final process occurs slowly from months to years and it is facilitated by the loads bones are bearing during ordinary daily activities [34], [36], [37].

According to the description of fracture repairing, an important stage of bone healing are the first 2 weeks in which revascularization occurs and needs to be guided. After that, in the period from 4 to 6 weeks, the formed callus is very weak in terms of resistance to movement. Thus, fracture requires adequate protection in the form of the incorporation of an artificial bone replacement. The process of the bone-material integration is shown in Table 2. This definition leads to consider highly relevant the effects of the mechanical stimuli on bone growth. Hence, the processes of new biological tissue creation demands an artificial substrate in order to guide tissue growth.

TABLE 2
Bone-implant integration process

| Time | | Process | Involved Cells |
|---|---|---|---|
| 0-2 | days | Clot formation | Platelets |
| 2-3 | days | Hematoma stabilization | Immunologic cells |
| 3-10 | days | Hematoma organization | Mesenchymal cells |
| | | Mesenchymal cells orchestration | Osteochondro- |
| | | | progenitor cells |
| | | Osteoblast differentiation | Preosteoblast |
| | | Production of local factors | Osteoblast |
| | | Osteon synthesis | Osteoblast |
| 1-3 | weeks | Osteon Maturation | Osteoblast |
| | | Calcification | Osteoblast |
| • | weeks | Bone tissue maturation | Osteocytes |
| >4 | weeks | Bone remodeling | Osteocytes |

### Stress Shielding

It was proposed that mechanical stimuli would modify bone geometry in a way that tended to prevent structural failure of skeletal tissues [37]. Thus, bone is a dynamic tissue which renews itself during normal function for adapting itself to mechanical loads. In this section, the constitutive model for adaptive bone remodeling proposed by Hazelwood et al. [39] is described in order to illustrate the differences in remodeling behavior of bone when it is placed in disuse or overload. This model is appropriate to evidence the reduction in bone density as a result of normal stress removal by an implant, phenomenon which is usually termed stress shielding.

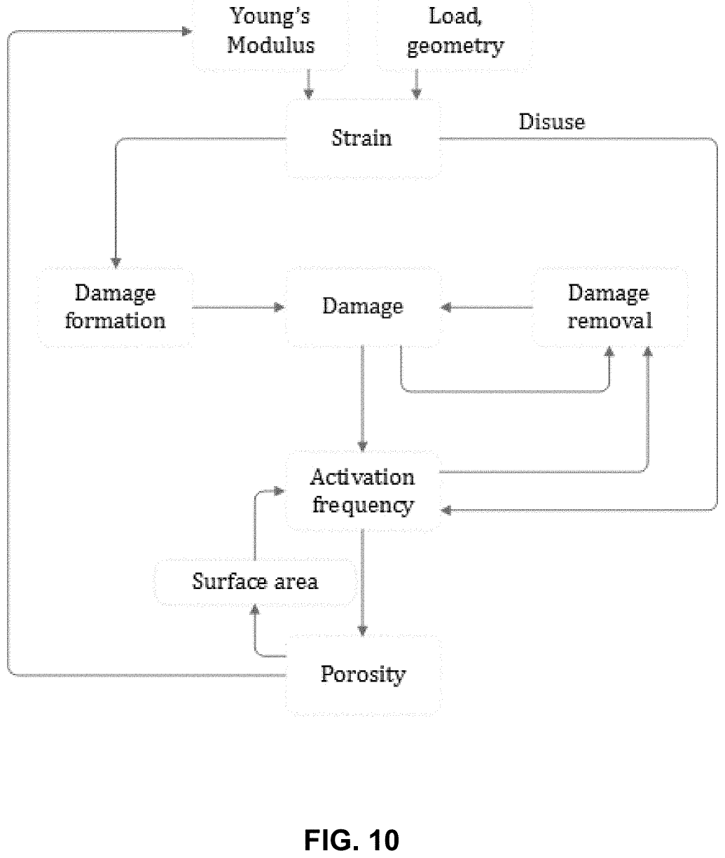

The model simulates porosity and bone elastic modulus changes by internal remodeling activation as a response to an altered mechanical loading. Schematic representation is shown in FIG. 10. There, the activation is caused by damage or low strain and the porosity is function of the activation frequency. The two feedback loops indicate that the more increased remodeling results the more increased are porosity, strain, and damage formation. In the activation case, spatial and temporal recruitment of cells that form and resorb bone are orchestrated. It is important to highlight that this group of cells orchestrated, responsible for remodeling activities, has been termed BMU (basic multicellular unit) [40].

Based on FIG. 10, porosity (P) change rate is a function of the activation frequency of bone remodeling which is divided in bone resorbing (Q_{R}) and bone refilling (Q_{F}) for each BMU. According to Martin [40], the BMU forms a cylindrical union about 2000 μm long and 150-200 μm wide. This canal progressively penetrates the bone at a speed of 20-40 μm per day. Osteoclasts have the ability to resorb fully mineralized bone, an activated osteoclast is able to resorb 200,000 μm³ per day which would be the amount of bone formed by ten generations of osteoblasts [41]. Then, osteoblasts, the cells within bone which provide the extracellular matrix and control its mineralization, will fill the canal to produce a renewed bone.

Hence, as a concordance between Martin and Hazelwood, the porosity chance rate is function of N_{R} and N_{F} the cells population density of resorbing and refilling, respectively. And it is also function of Q_{R} and Q_{F}, which are supposed to be linear in T_{R} and T_{F}, the resorption and refilling periods. The populations N_{R} and N_{F} are defined by integrating over T_{R}, Tᵢ (reversal time) and T_{F} time periods of the activation frequency (fₐ). Then, Hazelwood assumed fₐ as a function of disuse and damage. And, due to BMUs must start on the bone surface, fₐ is also related with surface area (S_{A}). The complex of equations that govern the model are shown in [37], [39].

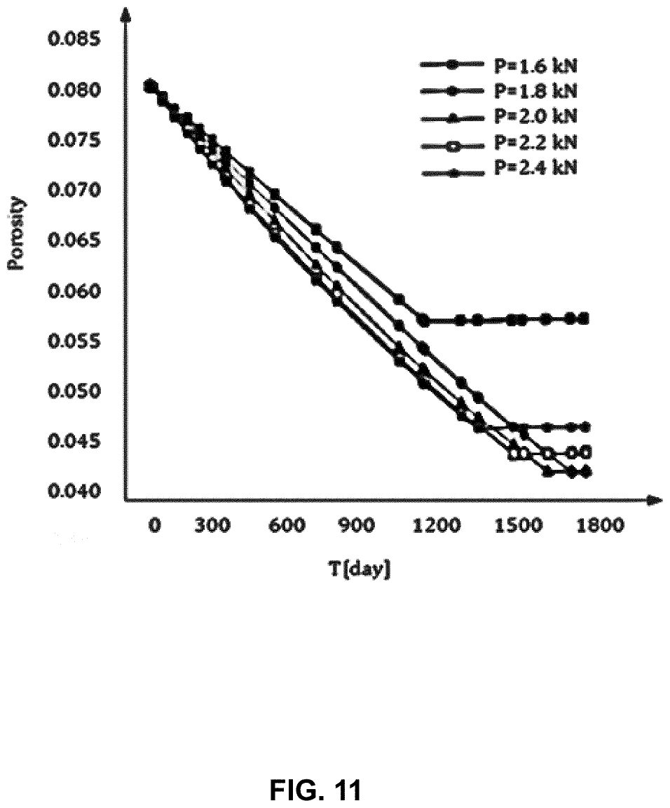

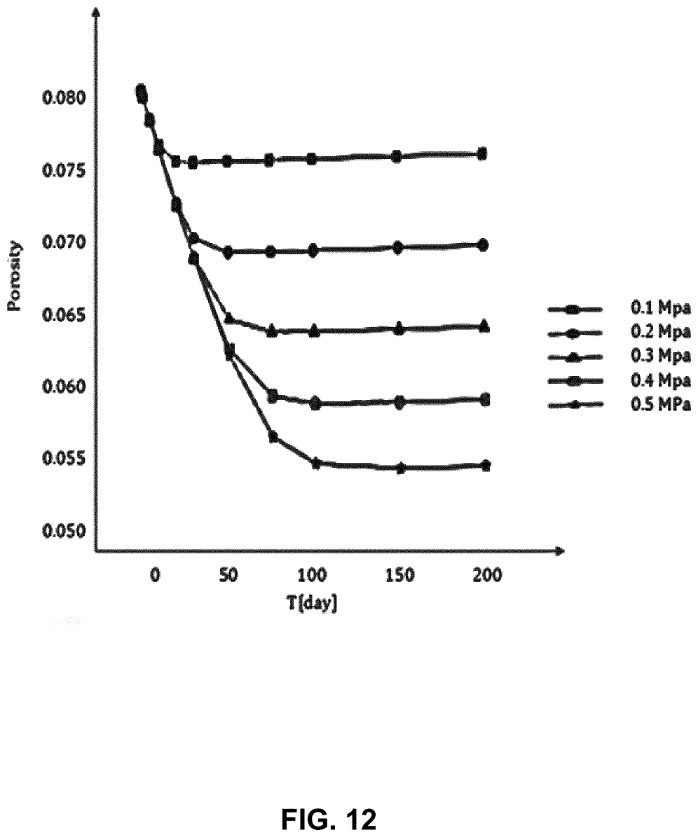

To illustrate de variation effect of axial stress, transverse stress, and a pin stiffness on bone porosity, numerical implementations of the model are described in [37]. The applied loadings for the effect of axial stress calculus were P=1.6, 1.8, 2.0, 2.2 and 2.4 kN, for the influence of transverse loads on bone porosity the stresses applied were P=0.1, 0.2, 0.3, 0.4 and 0.5 Mpa. Similar behaviors can be seen in both cases. The results in FIGS. 11 and 12 show that axial stress can strengthen a bone tissue by depositing new skeletal material. Then, the elastic modulus increases resulting in a decrease of strain on the bone structure. However, when the axial loading is high enough, a further increase in load has a very small effect on the remodeling process. Insufficient capacity of the body to produce osteoclast could be the principal responsible factor. Thus, the axial loading must have an upper limit which is still an open question [37].

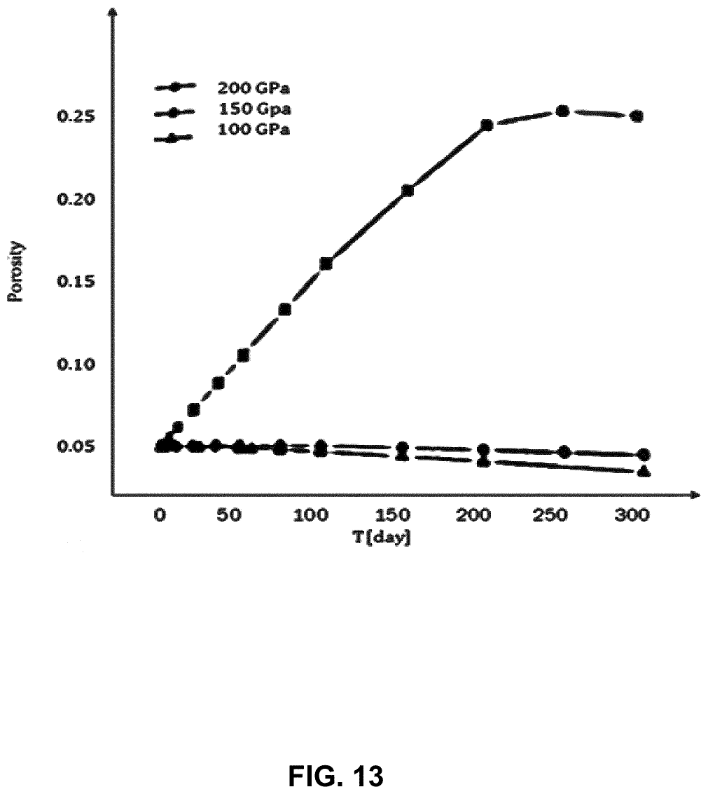

The effect of pin stiffness on the bone porosity change was calculated with the following values from Young's modulus E=100, 150 and 200 GPa. The change of bone porosity as a function of the time was plotted in FIG. 13. It is evident that when the E=200 GPa, the porosity increases as time progresses, while porosity remains stable when E=150 GPa or below.

This is the numerical prove of stress shielding of bones caused by implants, the fixation device is bearing a large portion of the load that would normally be bore by the bone and a reduction atrophic remodeling can be induced. Moreover, the Young's modulus for bone is E=10-30 GPa [20], [42] which means that the behavior observed for E=150 GPa pin is the expected for a E=˜90 GPa device. The natural purpose of raised remodeling rate is to eliminate “unnecessary” bone mass, remove damage or reorganize tissue. As a consequence of this bone negative feedback, remodeling reduces the bone porosity and can weaken the skeletal structure. This is therefore essential to understand the remodeling process, as a fundamental biological process in the design of a device for bone replacement.

### Introduction to Mg

### Chemical and Physiological Characteristics

Mg is the eight most common element on Earth [43] and is a chemical element in the group 2 (alkaline earth metal) within the periodic table. Its atomic number is 12 and its common oxidation number +2 [44]. Positively charged Mg2+ is able to bind electrostatically to the negatively charged groups in membranes, proteins and nucleic acids [45]. Mg is a highly reactive metal. Thus, security precautions must be taken when manipulating it. A summary of the physical properties of Mg is shown in Table 3.

TABLE 3
Physical properties of Mg

| Property | Value | | Property | Value | |
|---|---|---|---|---|---|
| Atomic | 12 | | Boiling | 1107° | C. |
| number | | | point | | |
| Atomic mass | 24.305 | g · mol −1 | Thermal | 155 | W/(kg K) |
| | | | conductivity | | |
| Density | 1.74 g · cm−3 at 20° C. | | Electrical | 4.45 | μΩ cm |
| | | | resistivity | | |
| Meting point | 650° | C. | Heat of | 25.1 | MJ/kg |
| | | | combustion | | |

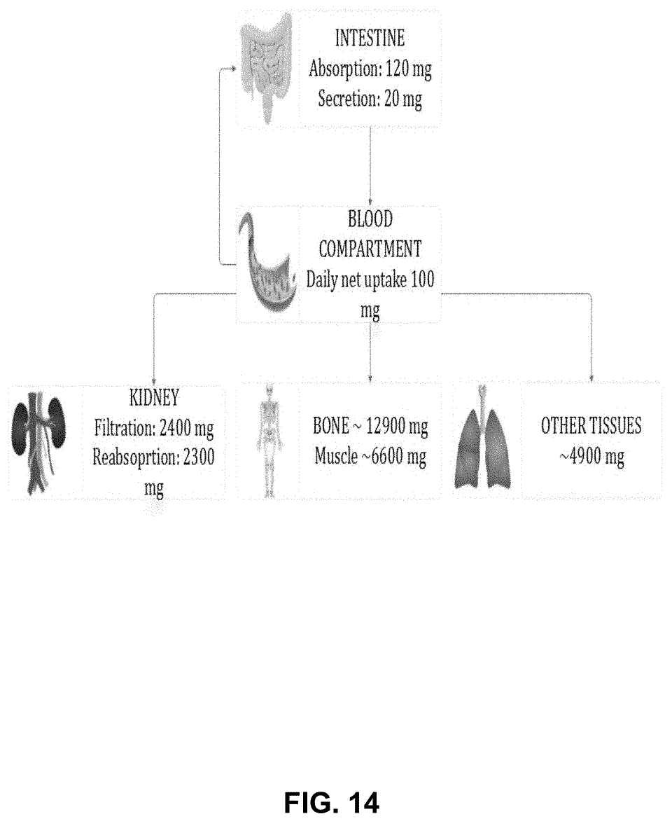

Mg is the fourth most abundant mineral in the human body. With about 760 mg at birth and increases up to 25 g to 35 g in the adult, per 70 kg bodyweight [17]. Approximately 50% of Mg is found in bone, the other half is predominantly inside the cells of muscles and soft tissues, where Mg is the most abundant intracellular divalent cation [47]. Only 1% of Mg is found in blood and the body works very hard to keep this percentage constant. The normal concentration of Mg in blood serum is 0.73-1.06 mmol/L [45], [47]. The distribution of Mg concentration in a healthy adult is shown in FIG. 14. The total body Mg concentration in the average 70 kg adult with 20% fat is 1000 to 1120 mmol or 20 to 28 g [48].

Mg is fundamental in a lot of biochemical reactions in the human body where it acts as co-factor in many enzymatic responses. Intracellular Mg is mainly bound to nucleic acids, ATP, phospholipids and proteins which are of central importance in the biochemistry of the cell, and particularly in energy metabolism [45], [50], [51]. Mg is also regulator for more than 350 proteins [52]. It helps to maintain muscle and nerve functions, keep heart rhythm steady, supports a healthy immune system and keeps bones strong [53].

The majority of Mg in the body is absorbed via daily intake. The human diet demand for Mg is about 375 mg/day with adjusted dosages subject to age, gender and nutritional preferences [17]. Meanwhile, the control over excess in serum levels is dependent on intestinal absorption as well as kidney function. Mg excess is also regulated via storage in bone and it serves as a reservoir of exchangeable Mg [49], [54].

The chemistry of Mg is unique among the elements of biological relevance. Mg is crucial for man and it is required in relatively large amounts. Moreover, Mg has an important role in bone formation and can also be referred to as a natural Ca antagonist [49]. In addition to being an essential element for the human metabolism, it can be safely removed from the body via kidney or excretion. Mg is also considered to be biocompatible and non-toxic [19], [55]-[57]. Therefore, Mg has the potential to be an ideal biomaterial, especially for bone tissue engineering applications.

### Evolution of Mg as a Biomaterial

The history of Mg as a biomaterial started shortly after the first production of metallic Mg in 1808. At that time, the material was used as ligature wire for bleeding vessels based on Mg resorption [58]. Because the problem of controlling corrosion of Mg in vivo had not been appropriately solved, many surgeons preferred to use more corrosion-resistant materials and Mg was no longer studied [59].

Nowadays, Mg and its alloys have been introduced and researched for cardiovascular and orthopedic applications. The Mg-based devices provide temporary mechanical support to narrowed arteries or fractured bones during the healing and remodeling processes, and are eventually replaced by new arterial vessel or skeletal tissue. Cardiovascular Mg-based implants demonstrated in 2013 their biocompatibility and feasibility for clinical use [60]. A more detailed review of biodegradable metal stents can be found in [61].

There have been also critical reports on the use of Mg in bone applications, which are mainly based on in vitro testing [18], [62]-[68]. The first approach was in 1924 when the little action of Mg as connective tissue stimulant was published. The results led to the hypothesis that Mg possibly accelerates the new bone production [69], [70]. Years after, around 1948, the concept of alloying Mg with other elements to improve its properties, emerged when Troitskii and Tsitrinreported [62] successfully treating 34 cases of pseudarthrosis with a plate and screw made of an Mg—Cd alloy. The osteosynthesis material was resorbed completely, without any remaining dangerous element and also stimulated the formation of callus bone. A patent for alloying Mg with Cd to improve the corrosion resistance was filed in 1969 [71].

Since the return of interest for Mg in biomedical applications, from essentially no papers on the topic some years ago, there are currently 10-15 articles found in SCOPUS each week dealing with different aspects of Mg bioperformance. This revolutionary approach toward biodegradable metallic materials is being rediscovered. The development of Mg implants gained recent attention because it can change the paradigm of permanent fixations as there is no need for a secondary removal surgery. Neacsu et al. [18] wrote a state of the art of recent patents on Mg-based bone implants and there were no porous structures on the list of patents. However, up to now there is no clinical application of Mg implants, mainly due to the repetition of the same systematic errors in the research works.

Therefore, research on Mg changed to focus more on the effect of certain types of alloying element and adopting techniques for in vitro testing. For example, Kirkland et al. investigated the influence of the alloy elements in the dissolution rates [72], [73], and also worked hardly in the optimization of the methods for assessing the corrosion rate of Mg and Mg-based alloys [68], [74], [75]. The problems of in vivo resorption being too rapid, too localized, or unpredictable were handled by them. Thus, their research efforts resulted in possible candidates for biomedical implants with customizable dissolution rates.

Witte et al [76], [77], since 2005, started to explore the potential of Mg-based biomaterials, particularly Mg-based alloys containing Al, Zn and RE and the associated bone response. The results showed high mineral apposition rates and an increased bone mass around the Mg implant. After that, the studies were mainly based on in vitro and animals testing. In that way, they assessed the role of biological environments on Mg behavior as biomaterial [78]-[80]. The products are in accordance with the results of Kirkland and demonstrate that Mg alloys can act as customizable materials which should be designed based on consideration of microstructure and localized environment degradation processes.

Kim et al. investigated the influence of Ca addition on corrosion of Mg in vitro by varying the percentage of Ca added into the alloy [81]. Likewise, Huan et al. compared the degradation behavior and cytocompatibility of different Mg—Zn—Zr alloys [82]. Zhang et al. worked hardly on assessing the effects of the binary Mg—Zn based alloys on the cells toxicity and morphology [83]-[86].

Concurrently, studies have explored techniques to slow down the corrosion rate of Mg implants. Several surface modifications have been proposed including microarc oxidation [87]-[89], calcium phosphate and hydroxyapatite coatings [90], fluorinated coating [91], polymer coatings [92], [93], heat treatment [94], ion-beam assisted deposition (IBAD) [95], and the list is still increasing.

### Development of Mg-Based Porous Structures

### Porour Metal Based Regeneration of Bone

In traditional orthopedic treatments, the stability of the bone fractures was principally achieved by using fixation and bone grafts. However, this paradigm is being changed due to the limited efficacy of the conventional strategies for large bone healing. Hence, the idea that the improved fixation can be achieved by bone tissue growing through a porous matrix of metal came into being, bonding in this way the implant to the healthy tissue [96]. Furthermore, if extensive vascularization, and body fluid transport through the porous matrix is possible due to an established pore interconnectivity, bone ingrowth can be activated. Moreover, porous materials have low Young's modulus, depending on the porosity. Therefore, moduli can be designed to match the mechanical properties of bone [97].

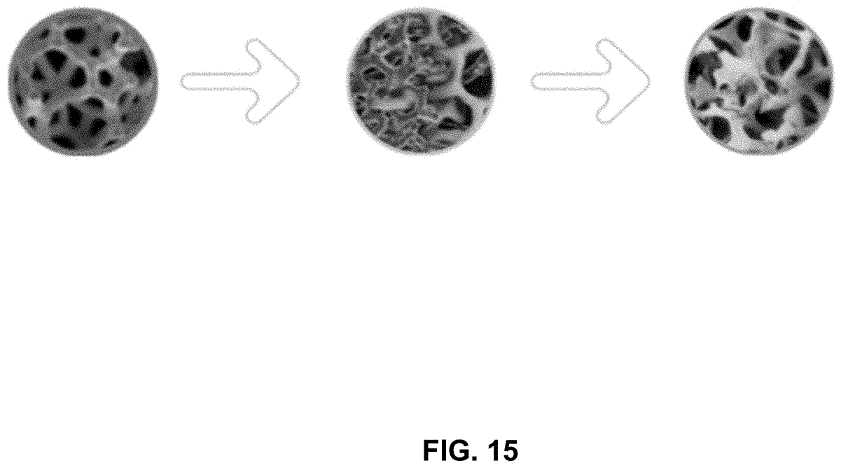

As it was described herein, bone has potential regenerative characteristics and most injuries can heal with conventional conservative therapy or surgery as long as skeletal continuity has not been disrupted. However, insufficient blood supply, infection of the bone or the surrounding tissues, and systemic diseases can undesirably influence the fracture healing. Also, complete repair is unlikely if the defect reaches a critical size and natural bone tissue is not able to regenerate itself across the gap [98]. In these cases, an optimal bone scaffold that replicates the architecture of the cancellous bone (as shown schematically in FIG. 15) to provide guidance and support during tissue development is desirable.

Although there are significant recent research efforts, there remain challenges to the effective implementation of Mg-based biomaterials. The most of these challenges are related to corrosion rate and morphology. Mg alloys offer excellent properties with regard to application as degradable implant. For bone implants, it is even more desirable to use porous materials. However, the preparation of high-porosity Mg implants has been difficult so far.

Several researchers have used terms to describe the biological events in porous implant stabilization and bone healing processes. In consensus, osteoconduction means that the bone grows on a substrate, thus, this term refers to the situation where bone can grow on the surface of an implant [99]. The term bone ingrowth refers specifically to bone formation within a porous surface structure [100]. Therefore, bone ingrowth requires osteoconductive surfaces for leading to a successful osseointegration of the implant.

Even though ceramics have good corrosion resistance, the general opinion is that ceramic-based porous implants, thus far, cannot be employed in load-bearing applications, due to their inherent fragility. Similarly, polymer-based porous devices are not able enough to support the mechanical forces present in a bone replacement surgery. These approaches conducted research labors to focus on porous metals for bone healing due to their superior characteristics of fracture and fatigue resistance, which are required for load-bearing applications [97].

Two kinds of porous metals or metals foams exist depending upon the fabrication methods: closed-cell and open-cell [101], [102]. In a closed-cell foam, pores are surrounded by a material wall and each pore is completely enclosed by a wall of metal. In an open-cell foam pores are connected to each other in space, allowing tissue and vascularization to infiltrate the foam and anchor to it.

Depending on the level of interconnectivity between the pores there are two distinct recognized types of porous implants [97]: 1) solid substrates coated by pores, and 2) porous materials. Implants with solid interiors and porous coating, type 1), are more suitable when the porous metal by itself does not provide sufficient mechanical strength to sustain the physiological loads, for instance, dental implants and joint arthroplasty implants. Also, there are several implant applications which can potentially use porous-type implants: spinal fixation devices, fracture plates, wires, pins and screws, artificial ligaments, attachments, maxillofacial implants, and bone graft materials to fill tumor defects [97].

The geometry of porous scaffolds in the case of bone substitution demonstrated having significant influence on the cellular response and the rate of bone tissue regeneration [103]. As described in section 2.2, there is a consensus about the importance of vascularization in bone regeneration. Thus, in order to complete the vascularization of scaffolds, material chemistry and macro and micro-structural properties ought to be optimized. The rate of vascularization depends on pore size, porosity, pore interconnectivity, and volume ratio [104]. A large surface area can lead to improved cell attachment, whereas highly porous scaffolds favor vascularization and nutrient interchange. Therefore, scaffolds for osteogenesis should mimic bone architecture, structure and function in order to enhance integration into surrounding tissues [33]. The minimum pore size required to regenerate mineralized bone is considered to be 100 μm [105], smaller pores result in ingrowth of unmineralized tissue, and even smaller ones are penetrated only by fibrous tissue [106]. Appropriate pore sizes for bone tissue engineering are reported to be >200 μm [107] while rapid vascularization of matrixes apparently requires a pore size greater than 500 μm [32], [108]. Hence, the general pore size for bone with high vascularization potential should be between 200 and 900 μm [32].

Pore size is not the only important parameter in a foam for bone healing process. Bone ingrowth should not be affected by the pore dimension as long as the assembly is fully interconnected [106], [109]. Highly porous foams could fail to support vessel ingrowth because of deficient communication of pores among each other. Thus, interconnectivity of pores also plays an essential role in the healing process. Furthermore, higher porosity is usually associated with greater bone formation [110]-[113].

The current development requirements which need to be met in order to carry out a bone substitute which can closely mimic the bone tissue microenvironment, have been briefly reviewed in this section to provide a reference for the remaining part of this thesis, with the aim of revealing a new alternative to support the bone tissue healing.

### Manufacturing Routes of Metallic Porous Structures

The advantages of metals that present a surface or bulk porosity have led to conduct systematic research aimed at clarifying the fundamental aspects of interactions between porous metals and hard tissue. In this section some of the main methods for fabricating fully porous metals are outlined. A summary of the fabrication methods is shown in Table 4.

The fabrication of porous materials has been actively studied since 1943, when Sosnik [114] tried to generate pores in a solid Al by adding Hg to the melt. At present, there are a number of methods used to fabricate closed-cell foams. Two general routes to generate porosity should be highlighted: melting and powder metallurgy. Moreover, the fabrication of open-cell porous metal implants can be divided into three categories according to the state of the metal: 1) liquid metal, 2) solid state, 3) metal vapor, and gaseous metallic compounds [97], [115].

TABLE 4
Fabrication methods for porous metals and their categorization
according to the pore distribution and the state of the metal

| | Porosity | |
|---|---|---|
| State of the metal | Closed-cell | Open-cell |
| Solid | | Sintering of powders and fibers |
| | | Hollow sphere structures |
| | | Combustion synthesis |
| Liquid | Gas injection | |
| | Blowing agents | |
| | Spray forming | Spray forming |
| | | Replication |
| | | Casting using polymeric foams |
| | | Rapid prototyping |
| Other | | Electro-deposition |
| | | Vapor deposition |

The first category of metal state consists in the creation of a foam from liquid metal. At melting point, the bulk metal is foamed directly or indirectly using space holders. There are two main ways to foam metallic melts directly: by injecting gas into the liquid from an external source, or causing gas formation in the molten metal by admixing gas-releasing blowing agents to the liquid [115].

Other manufacturing route consists in the partial densification during sintering of metal powders and fibers. This technique is identified as powder metallurgy because the starting materials are powders, which they become liquid metal [115]. This fabrication route is used in the production of porous surfaces and porous metallic implants. In this process the production of the scaffolds is obtained by mixing metal powders with a blowing agent, and binding and sintering the metal powders [118]. During the sintering process, the powder particles are bonded to each other due to the high-temperature. Nevertheless, there is only a minor change in the particle shape.

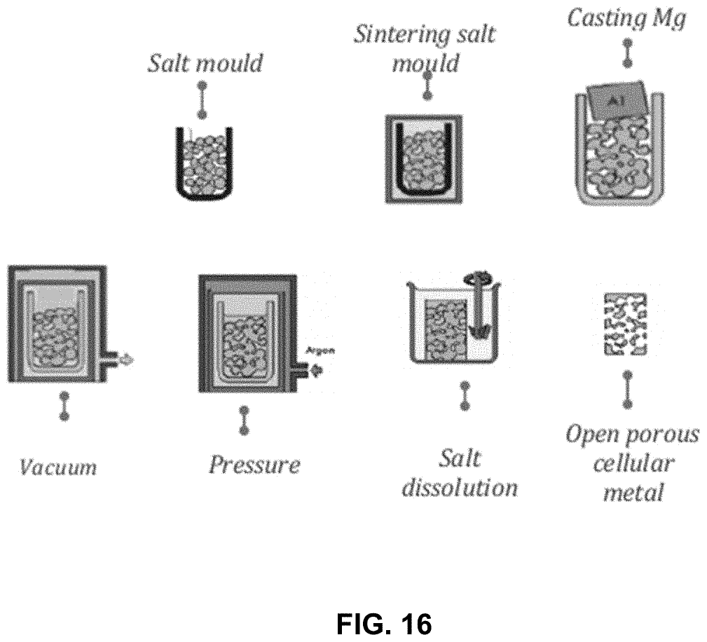

Replication process is a manufacturing method which consists in processing a preform that can be a sintered salt or a polymer foam impregnated with plaster slurry. The preform is then permeated with liquid metal, which infiltrates the free spaces between the grains. At the point of solidification the salt preform is dissolved in water and the spaces of the original preform are mostly copied. This process results in an open-cell almost fully interconnected foam [117]. Each step of the replication process is shown in FIG. 16. In the case of polymer foam, the template is filled with a slurry of a more heat-resistant material. After that, the polymer is removed by heat and liquid metal is casted into the cavities reproducing the original template structure [115].

Both replication process and space holder method are known to be the methods which can produce scaffolds with the greatest porosity. The difference between replication process and space holder techniques is that the free space between the salt grains is filled by fine metal powder instead of liquid metal. Therefore, the space holder process begins by mixing the metal powders with a space holder material and is followed by the compaction of the mix in order to form a green body. The resulting pellet is then subjected to a dissolution process which is designed to remove the space holder [97], [115].

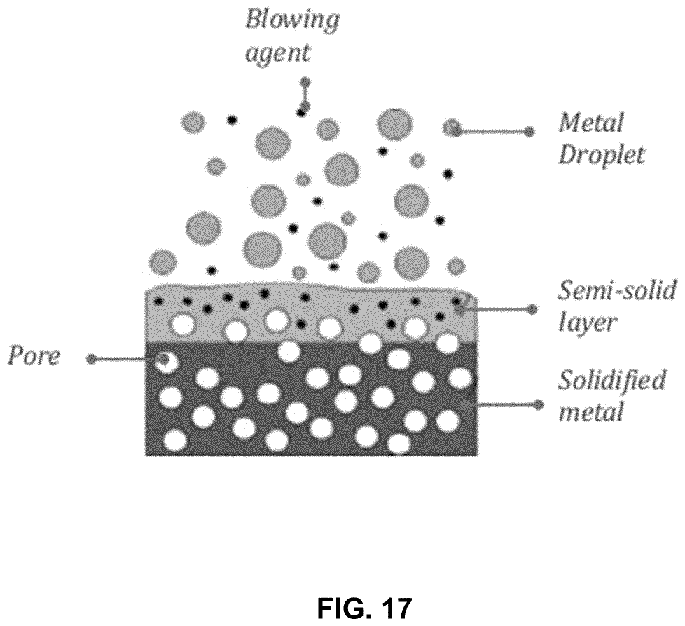

Spray forming is usually employed to create porous surfaces on solid cores; this combination of properties usually cannot be obtained by conventional casting methods. A schematic description is given in FIG. 17. Briefly, the liquid metal is continuously atomized and creates a spray of fast flying small metal droplets. The precipitations are collected and grow on a substrate to generate porous structures in a given shape [115], [118].

Metal foams can also be made from gaseous metal; two examples of porous structures obtained from metal gas will be highlighted here. The first one is the gas entrapment technique. In this method, metal powders are compressed into solid precursor materials with gas cavities trapped inside the material. This is followed by the heating process, in which pressure generated by heated gas expands the metal [44]. In the second technique, a solid precursor, as the polymeric template mentioned in the replication process, is required to define the geometry of the scaffold. Metal vapor in a vacuum chamber can be produced and used to condense on the cold mold. The condensed metal covers the polymer and forms a film. Finally, the thickness of the film is defined by the density of the vapor and the exposure time [115].

### Manufacturing of Porous Mg Scaffolds

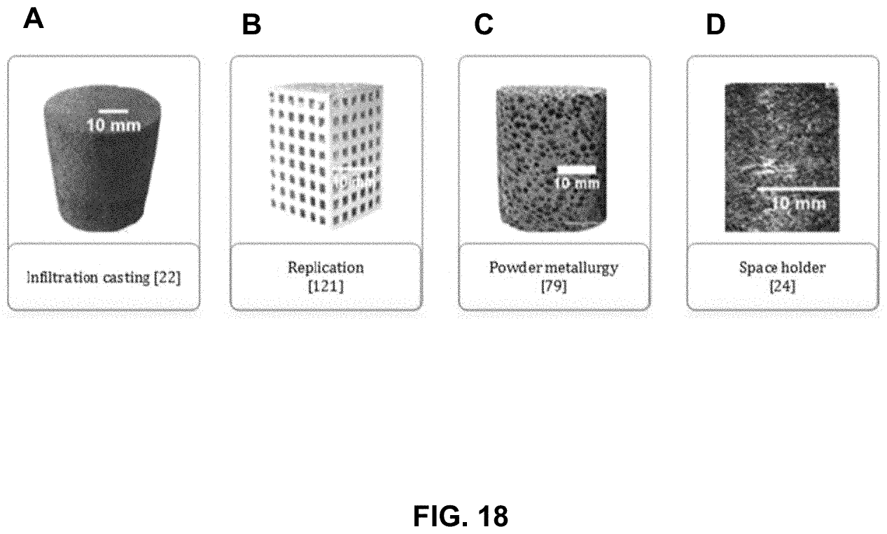

Some of the techniques described in herein have been employed by the researchers to fabricate Mg foams for tissue engineering. For instance, powder metallurgy [25], [65], space holder [23], [26], [119], gas eutectic unidirectional solidification (GASAR) [120], and infiltration casting [22], [24], [79], [121]. The properties of some porous metals obtained are given in Table 4, and the final structure is shown in FIG. 18.

TABLE 5
Properties of porous Mg devices

| | | | Pore | Yield | |
|---|---|---|---|---|---|
| | | Porosity | size | stress | |
| Alloy | Method | [%] | [mm] | [MPa] | Ref. |
| AZ91E | Infiltration | 68 | NR | 3.62 | [22] |
| | casting | | | | |
| WE43 | Infiltration | 39 | NR | 8.67 | [22] |
| | casting | | | | |
| ZM20 | Infiltration | 43 | NR | 3.48 | [22] |
| | casting | | | | |
| ZWM200 | Infiltration | 69 | NR | 2.31 | [22] |
| | casting | | | | |
| ZXM200 | Infiltration | 44 | NR | 8.66 | [22] |
| | casting | | | | |
| Pure Mg | Powder | 80-40 | 0.75-1.75 | 0.9 | [25] |
| | metallurgy | | | | |
| Pure Mg | Space holder | 54.4-70.4 | 0.6-0.9 | 3.57-8.65 | [23] |
| Pure Mg | Infiltration | 67 | 1.7 | 2.5 | [24] |
| | casting | | | | |
| Pure Mg | GASAR | 28 | 0.170 | 23.9 | [120] |
| AZ91 | Infiltration | 25 | NR | 7 | [122] |
| | casting | | | | |

(NR: Not reported)

Trinidad et al. [22] investigated porous Mg processed by infiltration casting with porosity ranging from 30 to 60%. They described the effect of the process parameters on the properties of the foams with five Mg-based alloys. The lowest achieved porosity was 30% with the ZWM200 alloy. The highest porosity was 69% which is the typical porosity in the Al foams obtained by this method. The mechanical properties of the scaffolds were directly related to the porosity achieved. It can be seen, in the first five alloys in Table 5, that there is an inverse relationship between the porosity and the mechanical response. Moreover, Hao et al. [25] showed that the morphology and sizes of the pores can be controlled by selecting an appropriate carbamide particle; furthermore, a tailored porosity can also be obtained by varying the volume fraction of the space holder.

Wang et al. [23] developed a weakly corrosive and highly flexible salt mixture space holder to fabricate Mg foams by infiltration casting. Their results showed foams with stress-strain behavior dependent on the porosities and the relative densities of the foam samples. Osorio-Hernandez et al. [24] also used replication process to obtain Mg foams and found that increasing the pore size, the relative density decreased, while the porosity increased. Hence, the smaller pore size (1 mm) and the lower porosity percentage (67%) exhibited the highest mechanical properties. Jiang and He [119] proposed the use of titanium wire as space holder, and obtained a porous structure as interconnected pipe-like pores (FIG. 18D). The pores geometry was individually controlled to obtain mechanical properties comparable to those of the cancellous bone.

In general, the fabrication of porous structures based on Mg alloys has been limited, mainly because of the high reactive potential of this material, hence, the complications with the sintering. In the current state of this review, all the processes described require modification of the equipment and the procedures already standardized for other metals. Since Mg is highly flammable, processing of the material is normally performed in vacuum or in an inert gas environment. There is a need for studies which focus their efforts on developing a reliable, safe and low-cost method for manufacturing porous Mg foams. Degradation rate control of Mg implants by surface modification

Chemically active Mg possesses a strong electronegativity and standard potential of 2.3V [123]. Hence, the degradation rate is one of the biggest problems in its real applications. In the recent years, the corrosion mechanism of Mg alloys has been actively researched. Therefore, many solutions, from the chemical and physical aspect, have emerged. This section provides a summary about research progress of surface modification of Mg-based biomaterials.

Wu et al. [124] wrote a review describing the prospective methods for surface modification of Mg alloys. They classify the methods into five categories: I) Substrate-involving coatings. It means that a portion of the Mg substrate participates in the preparation process and is eventually converted into deposited coating; II) Ca—P-based coatings. Since Ca and P are the main elements in bone tissue, they have been widely used to promote osteoconduction; Ill) Polymer-based coating, using polymers approved for human clinical applications; IV) Composite coatings, various combinations such as ceramic/ceramic and ceramic/polymer have been proposed; V) Ion implantation, this technique involves a process in which ions are accelerated and impinge on the surface. This method provides the possibility of introducing different species into a substrate independently from thermodynamic limitations such as solubility.

Microarc oxidation (MAO) or plasma electrolytic oxidation (PEO) is one of the ways to fabricate substrate-evolving coatings on Mg-based materials. Pan et al. [125] used this technique to fabricate Ca—P ceramic coatings on pure Mg, Mg-0.6Ca, and Mg-0.55Ca-1.74Zn. They studied the coating formation, and the growth and mineralization mechanism. Some conclusions can be drawn from their results: the porosity and microcracks of the coating are two important factors affecting the electrochemical corrosion behavior. The movement of dissolution-precipitation balance depends on the coating corrosion resistance and apatite's forming ability.

Xiao et al. [126] prepared a Ca—P coating on AZ60 Mg alloy using phosphating technology. The degradation behavior was studied in vivo using coated and uncoated samples. Then, significant differences in mass losses and corrosion rates between uncoated samples and Ca—P coated samples were observed by micro-computed tomography.

Usually, Ca—P based coatings have relatively high dissolution rates in the biological environment and do not have long-term stability. Researches have emphasized in paying attention to the degradation rate in the initial healing stage. To meet the healing requirement, Li et al. [127] presented a study that addresses the deposition of a duplex coating by MAO treatment and polycaprolactone (PCL) coating to improve the corrosion resistance of pure Mg. The corrosion resistance of the complex was evaluated by immersion test. Their results revealed that PCL coating increased the corrosion resistance of Mg.

As another strategy, Wu et al. [128] implanted Zn ions, one of the vital elements in the human body, into pure Mg by using a cathodic arc source, at an accelerating voltage of 35 kV. The degradation rate in SBF decreased significantly. X-ray photoelectron spectroscopy (XPS) revealed galvanic effects between the metallic Zn-rich surface and the lower Mg matrix.

A description of some surface modification methods recently applied for Mg-based materials was presented. Nevertheless, the aim of the surface design is to alter the surface while not changing the bulk properties. Hence, the dynamic interface may be considered in order to provide the materials with the desirable mechanical properties, corrosion resistance, and biocompatibility during tissue healing. Therefore, temporary surfaces that can better meet clinical needs and their application to biodegradable materials are expected to emerge.

### Conclusions and Perspectives

In this section and described herein, main concepts of bone tissue engineering are described. Also described are examples about the addressing of Mg behavior problems. Development of biodegradable implants has revolutionized the paradigm of hard bone replacements. Since the reported works based on Mg behavior had shown some successful results, it is promising but still challenging to obtain such a new kind of biodegradable implant.

### Biodegradable Mg-Based Alloys: Properties and Selection

### Introduction

Magnesium (Mg) alloys, when used in biomedical applications, have advantages over other materials such as ceramics, metals and traditional degradable polymers because of their mechanical properties. Metals are more suitable for load bearing applications in comparison to ceramics and polymers due to their high mechanical strength and fracture resistance [55]. The density values of Mg and its alloys are very similar to those of cortical bone while commercial biomedical materials usually used as bone implants, such as titanium Ti6Al4V, 316L stainless steel and synthetic hydroxyapatite have very different density values, as shown in Table 6. These density differences may produce stress shielding and micromotions at the bone-implant interface, leading to disproportionate interfacial mechanical stress. This phenomenon may guide to the growth of fibrous tissue and, finally, implant failure [129]. To address both restrictions, new materials with mechanical properties closer to those of the bone may be used. At present, strong requirements in bone repair and regeneration of bone defects are still to be met.

TABLE 6
Density values for materials commonly
used in biomedical applications

| | Density | |
|---|---|---|
| Material | [g/cm³] | Reference |
| Mg | 1.74 | [130] |
| Mg alloys | 1.75-1.85 | [130] |
| Cortical bone | 1.80-2.00 | [68] |
| Ti6Al4V | 4.47 | [68] |
| hydroxyapatite | 3.05-3.15 | [130] |
| 316L | 8 | [68] |

In order to ensure the safety of biodegradable materials, researchers have worked on the development of new types of Mg alloys with other transition metals, without toxicity or with low toxicity values, mainly including systems like Mg—Ca, Mg—Zn and Mg—Mn. Advances in the description of the properties provided by the new groups of alloys, have been described [55], [57], [67]. For instance, Mg—Ca alloys do not induce cytotoxicity in cells and reduce the corrosion rate compared to pure Mg. Furthermore, osteoblasts and osteocytes have shown high activity around Mg—Ca implants [131]. Zn is an essential element in the human body and has a strengthening effect in the alloys. Additionally, Zn can increase both the corrosion potential and toughness of Mg [132]. Other alloying elements such as Mn, Si and Ag have been studied to evaluate its biological behavior against to pure Mg. The lowest adverse effects have been found in Mg—X alloys systems [56].

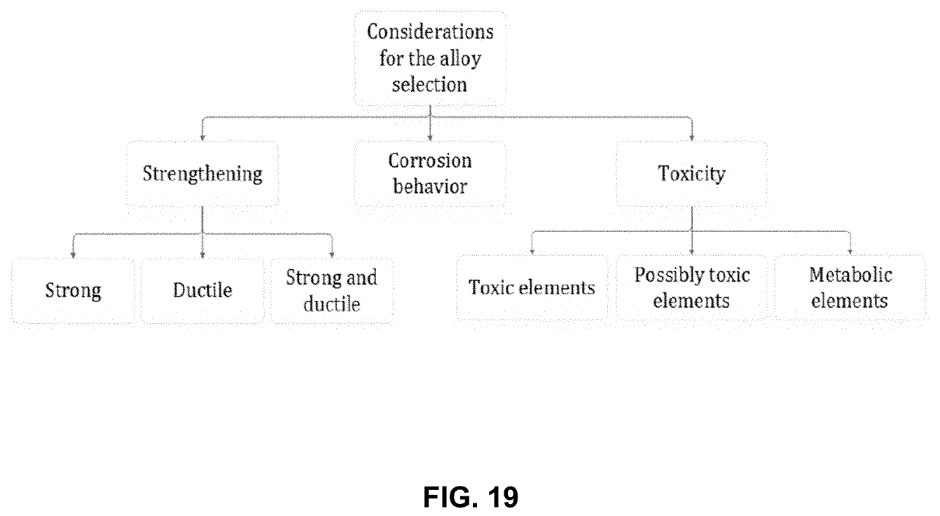

Mg and its alloys are lightweight metals, highly biocompatible and have mechanical properties similar to those of natural bone [55]. These materials have the potential to be osteoconductive and biodegradable replacements for load-bearing applications in the domain of hard tissues. However, the high corrosion problems in physiological environments have limited their use in the human body. For these reasons, a review of the Mg-based alloys used for orthopedic and tissue engineering over a period of 10 years is provided. The considerations for choosing the most appropriate Mg alloy are summarized in FIG. 19. A strategy for the accurate selection of the alloy is also described herein.

Corrosion Behavior

Biodegradable materials are designed to provide temporary support during the healing process and then progressively disappear. This structure requires materials that offer adequate mechanical properties and a degradation rate similar to the tissue healing rate. Hence, the key for the development of a biodegradable Mg-based implant is to bring the corrosive attack at the lowest possible rate. Alloying is one of the most effective ways to control degradability [19], [55].

The designation system of the Mg alloys generally follows the nomenclature of the American Society for Testing and Materials (ASTM) [133], [134] and uses a combination of letter and typical figure. Mg alloys can be divided in three main groups: pure Mg (Mg) with traces of other elements, Mg alloys containing Al and Al free alloys. From these groups, only the alloys that enable slow corrosion rate are useful for orthopedic applications.

Generally, elements can directly strengthen the mechanical properties of the alloy through different strategies such as precipitation hardening and grain refinement [56], [135]. The most common elements in Mg alloys are Al, Ca, Li, Zn and Mn. These elements can react with Mg or between themselves and form intermetallic phases. When these metallic phases are distributed within the grain boundaries or in the Mg matrix, they influence the corrosion behavior as described in Table 7. Some points can be summarized: elements such as Al, Mn, La and Nd improve the corrosion resistance of Mg alloys. For another elements such as Ca, Sr and Zn, the effect depends on the element concentration: if the concentration is high, the corrosion resistance is deteriorated, whereas when the concentration is low corrosion rate decreases. The corrosion resistance is always affected by the addition of Li.

The degradability of Mg-based alloys is an issue because of the acceptance of alloying elements by the human body. When the element concentration exceeds the tolerance limit, the corrosion rate is significantly accelerated [19]. Hence, the high corrosion rate of an Mg-based sample changes pH and concentration of ions, which have a negative effect on cell viability. Orthopedic materials need a period of 3 to 4 months from the callus formation to the new bone growth [136]. Unfortunately, most of currently investigated Mg alloys degrade too fast and the mechanical properties fall abruptly in the initial stage of corrosion. Erinc et al [137] suggested that, in order to keep the mechanical properties long enough, the degradation rate may be less than 0.5 mm/year in simulated body fluid. Pure Mg, in its compact form, has a corrosion rate of 2.89 mm/year. The corrosion rate of the most common Mg alloys is shown in Table 8.

TABLE 7
Effect of the alloying elements on Mg corrosion rate

| Element | Effect | Reference |
|---|---|---|
| Al | The microstructure of the alloys containing Al positively affects the corrosion | [65], [66], |
| | properties in simulated body fluid (SBF). The corrosion resistance increases in | [138], [139] |
| | proportion to the content of Al in the alloy. | |
| Ca | Excessive addition of Ca in Mg alloys deteriorates the corrosion resistance. The | [57], [66], |
| | concentration of Ca in Mg alloys should be less than 1%. The corrosion behavior | [67] |
| | of samples with small amounts of Ca was milder and more uniform that of the | |
| | samples with higher amount. | |
| Li | Li is more active than the Mg metal and that condition is reflected in its corrosion | [140], [141] |
| | resistance. Li improves corrosion resistance at concentrations below 9% in pure | |
| | Mg but significantly accelerates the corrosion rate with higher additions. | |
| Mn | Mn improves corrosion resistance in small amounts. A high concentration of Mn | [57], [131] |
| | causes deterioration in the corrosion resistance of the Mg-based alloys by forming | |
| | Al intermetallic phases containing Mn. Studies suggest that the addition of 1% Mn | |
| | enhances the corrosion resistance in alloys based on Mg—Mn. | |
| Zn | Zn inhibits the damaging effects of impurities like Fe and Ni. The corrosion | [55], [57], |
| | resistance improves with the addition of Zn. However, excessive addition of Zn can | [142], [143] |
| | impair corrosion resistance. The optimum content of Zn in Mg alloys may be at | |
| | least 5% | |
| Sr | Its influence on corrosion depends on the volume fraction, the optimum content is | [142], [144] |
| | less than 2%. | |
| La | The addition of less than 1%, results in a uniform corrosion. | [57] |
| Y | Its influence on the corrosion resistance is not clearly established. It depends on | [57] |
| | the alloy composition. In Mg—Y systems the corrosion resistance increases when | |
| | the concentration is >2%. | |

TABLE 8
Mg alloy corrosion rates

| | Corrosion rate | | | Corrosion rate | |
|---|---|---|---|---|---|
| Alloy | [mm/year] | Ref. | Alloy | [mm/year] | Ref. |
| Mg—1Zn—1.1Mn—1.0Ca | 0.01 | [145] | Mg—10Gd | 1.75 | [146] |
| Mg—3.09Nd—0.22Zn—0.44Zr | 0.01 | [147] | Mg—1Zn—1Ca | 2 | [83] |
| LAE442 | 0.2 | [56] | Mg—6Zn | 2 | [83] |
| Mg—Nd—Zn | 0.3 | [56] | Mg—1Al | 2.07 | [148] |
| WE43 | 0.3 | [149] | ZK60 | 2.2 | [150] |
| Mg—15Dy | 0.35 | [151] | Mg—1Zr | 2.2 | [148] |
| WEA31 | 0.4 | [149] | Mg—2Zn—1Ca | 2.3 | [83] |
| Mg—2Zn—1.5Y | 0.4 | [56] | Mg—1In | 2.32 | [148] |
| Mg—0.6Si | 0.4 | [131] | Mg—1Mn | 2.46 | [148] |
| Mg—0.8Ca | 0.5 | [1.31] | Mg—3Zn—1Ca | 3 | [83] |
| AZ31 | 0.5 | [138], [139] | Mg—4Zn—1Ca | 4.7 | [83] |
| Mg—8Y—1Br—2Zn | 0.7 | [56] | Mg—1Si | 5 | [148] |
| Mg—2Sr | 0.8 | [145] | Mg—5Zn—1Ca | 6 | [83] |
| Pure Mg | 0.9 | [55], [131] | Mg—1Ag | 8.12 | [131] |
| Mg—5.6Zn—0.55Zr—0.9Y | 1 | [150] | Mg—6Zn—1Ca | 9 | [56] |
| AZ91 | 1.3 | [138] | Mg—1Ca | 12.56 | [148] |
| AZ61 | 1.5 | [138] | ZM21 | 18 | [152] |
| Mg—1Ca | 1.7 | [148] | ZK61 | 26 | [83], |
| | | | | | [152] |

### Mechanical Properties

The therapeutic targets of the material configuration proposed in this thesis are load bearing orthopedic applications. Pauwels established the first ideas on how the tissue regeneration process is related to mechanical factors (see [153] for a review). According to his work, new bone formation during fracture repair occurred only under well stabilized mechanical conditions. The Mg alloys dedicated to this purpose may combine high strength with low Young's module, near to the bone, in order to prevent stress shielding. A description of this phenomenon is provided herein.

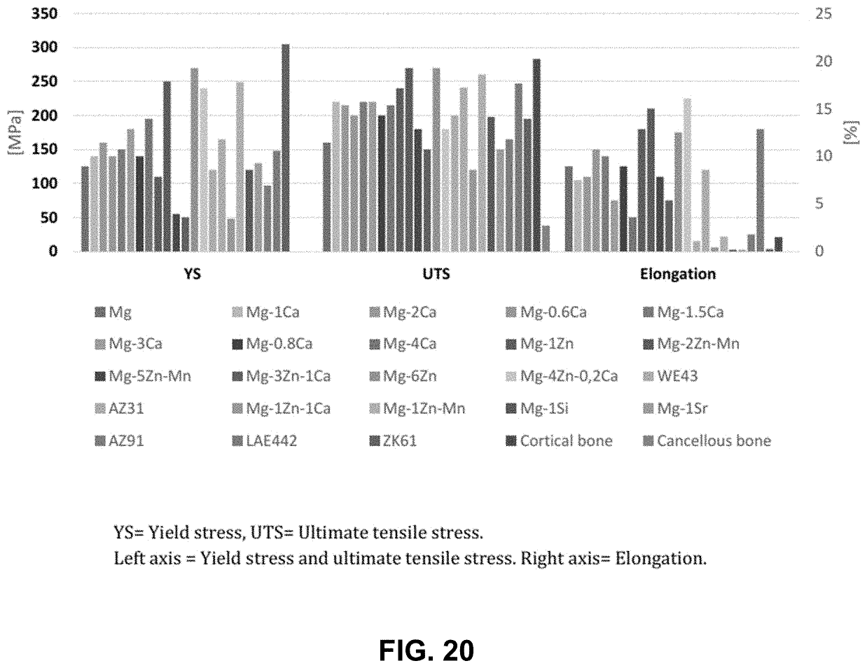

Erinc et al. [137] proposed some mechanical and corrosion requirements for bone fixations: corrosion rate may be less than 0.5 mm/year in simulated body fluid at 37° C., higher strength than 200 M Pa and elongation greater than 10%. A summary of the mechanical properties of the most commonly used alloy elements in biomedical applications is listed in Table 9. The mechanical integrity during the degradation is as important as the initial resistance. As illustrated in FIG. 20 Mg alloys show a large range of tensile strengths and ultimate elongation.

TABLE 9
Effects of the alloying elements on Mg mechanical properties

| Ele- | | | | | | Yield | |
|---|---|---|---|---|---|---|---|
| ment | UTS | Ductility | UCS | Hardness | Creep | strain | Castability |
| Al | ++ | ++ | | + | | + | + |
| Ca | + | − | + | + | ++ | | + |
| Li | − | + | − | | | − | |
| Mn | + | + | | | + | + | |
| Zn | + | − | | | | | − |
| Zr | + | + | + | | | ++ | |
| Sr | − | + | − | | + | + | |
| La | + | | | | + | + | |
| Nd | + | | | | − | + | |
| Y | ++ | + | | | ++ | ++ | + |

UTS = ultimate tensile stress,
UCS = ultimate compressive stress.
Effect coding:
++ = excellent,
+ = good,
− = bad

### Cytotoxicity

There are many considerations in the selection of the elements in an Mg alloy for biological applications, as shown schematically in FIG. 19. A basic consideration is the toxicity. Degradation products should be nontoxic and easily absorbed by the surrounding tissues or dissolved by excretion through the kidneys [132]. Alloys and alloying elements that have been reported for the manufacture of Mg-based biomaterials, can be considered in the following groups [56], [131], [154], [155]: I) well known as toxic elements: Be, Ba, Pb, Cd, Th; II) elements that may become toxic or cause allergy problems: Al, V, Cr, Co, Ni, Cu, La, Ce, Pr; Ill) elements that are part of human metabolism: Ca, Mn, Zn. Sn, Si, Al, Bi, Li, Ag, Sr, Zr.

Even though there are elements that are part of human metabolism, no metal can be consumed without restriction and many of the alloying elements may cause toxic reactions when consumed beyond its tolerance limits. The biocompatibility of alloys is influenced by the amount of free elements, and this factor is directly related to the corrosion rate of the alloy in the environment in which it is used [63]. This statement indicates that the alloying elements are safe if their release rate in the alloy is within the allowed limits. The maximum daily consumption for the elements commonly used in Mg alloys and normal levels of certain elements in the blood serum are shown in Table 10 and Table 11, respectively. It is clear that Ca has the higher limit, followed by Mg, Fe, Zn and Al, in that order.

TABLE 10
Toxic limits of frequently used elements in Mg alloy

| | Daily allowed | | Daily allowed |
|---|---|---|---|
| Element | limit [mg] | Element | limit [mg] |
| Be | 0.01 | Pr | 4.2 |
| Y | 0.016 | RE | 4.2 |
| Ni | 0.6 | Sr | 5 |
| Ti | 0.8 | Cu | 6 |
| Mn | 2.6 | Al | 14 |
| Sn | 3.5 | Zn | 15 |
| Ce | 4.2 | Fe | 40 |
| La | 4.2 | Mg | 400 |
| Nd | 4.2 | Ca | 1400 |

TABLE 11
Normal level in blood serum of elements
commonly used in Mg alloys

| | Normal level | |
|---|---|---|
| Element | in blood serum | Magnitude |
| Mg | 0.73-1.06 | mmol/L |
| Ca | 0.919-0.993 | mg/L |
| Al | 2.1-4.8 | μg/L |
| Zn | 12.4-17.4 | μmol/L |
| Mn | <0.8 | μg/L |
| Li | 2-4 | ng/g |
| Ni | 0.05-0.23 | μg/L |
| Fe | 5.0-17.6 | g/L |
| Cu | 74-131 | μmol/L |

It should be noted that the maximum dose of the elements listed in Table 10, refers to the intake dose allowed and might give an idea of the permissible amount in the human body. Nevertheless, the determination of the admissible limits also depends on the location of the implant, healing mechanisms and corrosion. For example, it is reasonable to expect different considerations for manufacturing vascular replacements, located in direct contact with the bloodstream, when compared to implants for orthopedic applications. Therefore, cytotoxicity tests can be used as quick indicators of the alloys behavior in the biological environment. The cytotoxic behavior of Mg alloys used in orthopedic applications is shown in Table 12.

TABLE 12
Cytotoxic effect of Mg alloys

| | | Culture | Cell | |
|---|---|---|---|---|
| | | time | viability | |
| Material | Cell line | (Days) | (%) | Reference |
| Mg | L929 | 4 | 65.7 | [55], [131] |
| Mg | NIH3T3 | 7 | 90.6 | [55], [131] |
| Mg | MC3T3-E1 | 7 | 87.5 | [55], [131] |
| Mg | ECV304 | 7 | 76.8 | [55], [131] |
| Mg—0.8Ca | L929 | 4 | 81 | [158] |
| Mg—1Ca | L929 | 4 | 81.8 | [1], [26] |
| Mg—3Ca | L929 | 4 | 55 | [55], [159] |
| Mg—1Zn | L929 | 4 | 111.8 | [55], [131] |
| Mg—1Zn | NIH3T3 | 7 | 114.1 | [55], [131] |
| Mg—6Zn | L929 | 4 | 100 | [55], [83] |
| Mg—1Zn—Mn | L929 | 3 | 100 | [55], [145] |
| Mg—1Zn—1Ca | L929 | 7 | 75 | [55], [160] |
| Mg—2Zn—1Ca | L929 | 7 | 70 | [55], [160] |
| Mg—3Zn—1Ca | L929 | 7 | 72 | [55], [160] |
| Mg—4Zn—3Al—0.2Ca | MG63 | 3 | 90 | [145] |
| Mg—1Si | L929 | 4 | 88.3 | [55], [131] |
| Mg—1Sr | MG63 | 5 | 84 | [55], [131] |
| Mg—2Sr | L929 | 4 | 83 | [158] |
| AZ31 | MC3T3 | 4 | 88.3 | [161] |
| ZK41 | MC3T3 | 3 | 80 | [162] |
| ZK61 | MG63 | 3 | 80 | [163] |
| Mg—Y—Ca—Zr | MC3T3-E1 | 3 | 75 | [164] |
| Mg—3Sn—0.5Mn | L929 | 3 | 65 | [165] |
| WE43 | L929 | 3 | 65 | [165], [166] |

Table 12 summarizes the viability of various cell lines cultured in extracts of Mg alloys. According to ISO 10993-5: 2009 [167], when there is a reduction in cell viability of over 30%, it is considered that there are cytotoxic effects. According to Table 12, pure Mg, Mg-3Ca, Mg-0.5Mn-3Sn and WE43 alloys have cytotoxic effects on L929 cells.

Mg and Ca are documented as to be biocompatible elements and have high allowed doses in the diet (Table 10). Besides different tolerance capabilities of the cell lines, the reason for these results can be attributed to the corrosion rate. Chen et al. [56] studied the influence of pure Mg with different corrosion rates obtained with different extrusion temperatures. The results confirm that the corrosion rate has significant influence on viability, adhesion and cell proliferation.

### Analysis of the Alloys and Selection Method

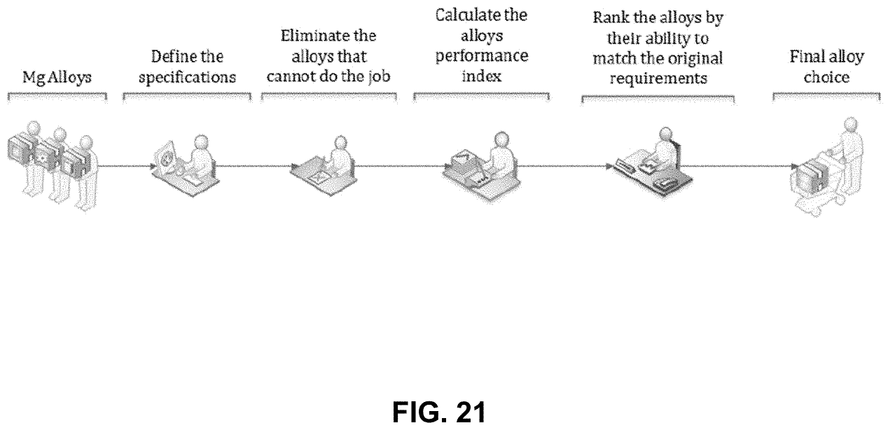

The sequence followed for the selection of the best Mg-based alloy is shown in FIG. 21. The specific considerations in the selection process are described below. Briefly, The first step is to specify the performance requirements of the component and broadly outline the main materials characteristics [168]. Then, certain classes of materials may be eliminated or chosen as probable candidates. Finally, the relevant alloys are ranked in order of performance and the optimal material is selected.

The selected alloy should satisfy its requirements in service. For bone replacement cases, the success of an implant for orthopedic applications is highly dependent on three major factors: The first and foremost requirement for the choice is its acceptability by the human body. A biomaterial used for body implant should have enough biocompatibility for long-term usage without rejection. Second, biodegradable implants may have the less corrosion rate possible and this is basically coupled with the third condition: mechanical properties close to those of human bones: yield strength of 104-121 MPa, ultimate tensile strength of 283 MPa and elongation of 1.07-2.10% [19], [169]. All of these requirements were included in the studied properties and pondered with corresponding values to obtain a global result.

The principal group of alloys was chosen by ranking corrosion behavior (Table 8) and cytotoxicity (Table 12). The properties of the selected alloys are presented in Table 14. The alloying elements which are part of the chosen alloys, were excluded due to the toxicological limits in Table 10. Therefore, no rare earths containing alloys were taken into account.

In the well-known Weighted Properties Method, for each material property a weight is assigned, depending on its importance. A weighted property value is obtained by multiplying the scaled value of the property by the weighting factor (a). The individual weighted property values of each material are then summed to obtain a comparative materials performance index (γ). The material with the highest γ is considered as the best option for the application. In the cases where numerous material properties are specified and the relative importance of each property is not clear, determinations of a may be mostly intuitive, which reduces the consistency of the selection [170], [171]. This is true especially when combining the numerical values of the mechanical and physiological properties. This problem may be solved by adopting a systematic approach to the determination of a and introducing the scaling factors, as described in [172].

To calculate a, using the Digital Logic Approach as described in [172], evaluations are arranged in a way that only two properties are considered at a time. Every possible combination of properties or performance goals is compared using a yes (1) or no (0) decision for each evaluation. For example, it can be seen in Table 13 that cytotoxicity (CC) is more significant parameter than yield stress (YS), therefore, CC has 1 and YS has a 0. The properties are listed in the left column, and comparisons are made in the columns to the right.

For the determination of the factors, each property is scaled, thus its maximal numerical value does not exceed 100. Equation 1 and Equation 2 were used for this purpose. Each time when a list of candidate materials is evaluated, each property will be taken into consideration only once. The highest value appreciated in the list is 100, and all other properties are scaled proportionally. Introducing the scaling factor facilitates the conversion of the normal values of each property of the candidate material into dimensionless scaled values.

Based on the selection method exposed above, the positive decisions and the pondering factors are listed in Table 13. Finally, the performance indices were calculated using the data summarized in the aforementioned tables, and the results are presented in Table 15.

For a given property, the scaled value B, for a candidate material will be:

$$B = scaled property = \frac{Numerical value of the property \times 100}{Maximum value in the list} \qquad ( Eqn 1 )$$

For properties where a low value is more desirable is calculated as:

$$B = scaled property = \frac{Minimum value of the list \times 100}{Numerical value of the property} \qquad ( Eqn 2 )$$

Then, the material performance index is:

γ=Σᵢ₌₁ⁿYᵢ∝ᵢ (Eqn 3)

Then, α calculated with the Equation 4:

$$α = \frac{Positive decisions}{N} \qquad ( Eqn 4 )$$

TABLE 13
Determination of the pondering factors for each property

| | | | | | | | Pondering |
|---|---|---|---|---|---|---|---|
| | | | | | | Positive | factor |
| Property | CC | YS | UTS | E | CR | decisions | (α) |
| Cytotoxicity | | 1 | 1 | 1 | 1 | 4 | 0.4 |
| (CC) | | | | | | | |
| Yield Strength | 0 | | 1 | 0 | 0 | 1 | 0.1 |
| (YS) | | | | | | | |
| Ultimate Tensile | 0 | 0 | | 1 | 0 | 1 | 0.1 |
| Strength (UTS) | | | | | | | |
| Elongation (E) | 0 | 1 | 0 | | 0 | 1 | 0.1 |
| Corrosion rate | 0 | 1 | 1 | 1 | | 3 | 0.3 |
| (CR) | | | | | | | |

TABLE 14
Properties of selected candidates

| | | | | | | CR |
|---|---|---|---|---|---|---|
| | | CC | YS | UTS | ε | [mm/ |
| No. | Material | [% Viab.] | [MPa] | [MPa] | [%] | year] |
| 1 | WE43 | 65 | 120 | 200 | 1.5 | 0.5 |
| 2 | Mg—0.8Ca | 81 | 147 | 198 | 8 | 0.5 |
| 3 | Mg—2Sr | 83 | 149 | 151 | 7 | 0.8 |
| 4 | AZ31 | 88.3 | 165 | 241 | 12 | 0.5 |
| 5 | Mg—1Zn—1Ca | 75 | 120 | 120 | 6 | 2 |
| 6 | Mg—1Zn—Mn | 100 | 249 | 260 | 22 | 0.3 |
| 7 | Mg—1Si | 88.3 | 120 | 198 | 2.5 | 5 |
| 8 | Mg—1Sr | 84 | 130 | 150 | 2.7 | 0.8 |
| 9 | Mg—1Ca | 81.8 | 140 | 220 | 10.5 | 2.08 |
| 10 | ZK61 | 80 | 305 | 195 | 3.5 | 26 |

TABLE 15
Hierarchization according to the performance index

| | | | | | | CR | |
|---|---|---|---|---|---|---|---|
| | | CC | YS | UTS | ε | [mm/ | Performance |
| No. | Material | [Viab. %] | [MPa] | [MPa] | [%] | year] | index |
| 1 | Mg—1Zn—Mn | 40.00 | 4.82 | 10.00 | 18.33 | 30.00 | 103.15 |
| 2 | AZ31 | 35.32 | 7.27 | 9.27 | 10.00 | 18.00 | 79.86 |
| 3 | Mg—0.8Ca | 32.40 | 8.16 | 7.62 | 6.67 | 18.00 | 72.85 |
| 4 | Mg—2Sr | 33.20 | 8.05 | 5.81 | 5.83 | 11.25 | 64.14 |
| 5 | WE43 | 26.00 | 10.00 | 7.69 | 1.25 | 18.00 | 62.94 |
| 6 | Mg—1Ca | 32.72 | 8.57 | 8.46 | 8.75 | 4.33 | 62.83 |
| 7 | Mg—1Sr | 33.60 | 9.23 | 5.77 | 2.25 | 11.25 | 62.10 |
| 8 | Mg—1Si | 35.32 | 10.00 | 7.62 | 2.08 | 1.80 | 56.82 |
| 9 | Mg—1Zn—1Ca | 30.00 | 10.00 | 4.62 | 5.00 | 4.50 | 54.12 |
| 10 | ZK61 | 32.00 | 3.93 | 7.50 | 2.92 | 0.35 | 46.70 |

Generally, in decision making the intuition is more common than numerical approaches. However, in biomaterials selection, in which there are numerous life-involving criteria, a more precise methodology would be required. When selecting materials, a large number of factors should be taken into account. During the design process, mechanical properties are of primary concern. However, in the biomaterials selection process one of the most important properties usually implicates corrosion rate and human body tolerance.

From the viewpoint of the corrosion mechanism, mechanical properties and cytotoxicity, this example summarizes the behavior of the most used alloys based on a survey of work accomplished over a period of the last 10 years. The ranking according to the performance index of the alloys is presented in Table 15. It can be observed that the materials with the best performance index are Mg-1Zn-Mn and AZ31 alloys. The Mg-1Zn—Mn alloy is on the first place, in spite of its mechanical properties which are different from those of human bone. This is due to the inclusion in calculations of the cytotoxicity and corrosion resistance, which are higher than in other alloys. The importance of those properties is accentuated by the pondering factor value, which have in this case the greatest value.

AZ31 alloy is on the second place according to the performance index. The AZ31 cytotoxicity and corrosion behavior are appropriate for bone implant and its mechanical properties are close to those of human bone. Moreover, the maximum allowed dose of the elements, listed in Table 10, shows a permissible amount of Al and Zn in the human body higher than that of the commonly used element Ti. Therefore, this is the alloy chosen as the most suitable for the aim of this work. Hence, it is recommended that Mg—Al alloy systems be used as experimental alloys to investigate the improvements of processing and surface modification technologies. A lot of data from long term toxic effects of Al are available today (see for a review).

### A Perspective of Alloy Design

The biological implications of alloying elements included in Mg bulk material as well as the improvement on corrosion, biological behavior and mechanical properties have been described. The research efforts during the last ten years have positively charted both mechanical and material properties. Mg-based alloys show a tendency for biocompatibility, thus, new biomaterials may be developed.

Mn is an essential mineral which plays a main role in the stimulation of multiple enzymes [174]. Mn is also predominantly used to enhance ductility and can be adopted to control the corrosion of Mg alloys given to the detrimental effect of Fe on the corrosion behavior [132]. As described in Table 7 the optimum content of Zn in Mg alloys may be at least 5% and small quantities of Mn could enhance the corrosion resistance of Mg alloys. Taking into account the aforementioned considerations, Mn and Zn, which have no toxicity, may successfully improve the corrosion resistance and mechanical properties of Mg-based alloys.

### Synthesis and Properties of Mg-Based Porous Materials

### Introduction

Bone morphology is composed by porous structures which create a natural environment with 50-90% porosity [33], and pore sizes of around 1000 μm in diameter, these structures are known as trabecular bone [175]. Cortical bone is surrounding trabecular bone, and is a solid structure with a series of voids (e. g. haversian canals), with a cross-sectional area of 2500-12,000 pmt resulting in 3-12% porosity [176]. As described in section 2.3, bone is at continuous state of remodeling with osteoblasts producing and mineralizing new tissue, osteoclasts resorbing it, and osteocytes supporting the created matrix [37].

Porosity is well-known as the percentage of air space in a solid [177]. Pores are essential for bone tissue healing because they allow migration and proliferation of cells, and vascularization, as well as nutrients interchange. Also, a porous surface improves the mechanical connecting between the implant and the host bone, providing greater mechanical stability and compatibility with this interface [96]. Pore size and porosity for bone application were reviewed in the section 2.5 in the context of mechanical properties and type of bone formation.

Highly porous foams, completely communicated, are desired for the material configuration proposed in this thesis. The minimum pore size necessary to regenerate mineralized bone is considered to be 100 μm [33], [105]. Some authors [33], [107] recommended pore size>300 μm for enhancing osteogenesis. Furthermore, as outlined above, the trabecular bone pore size of around 1000 μm. Hence, the pore size range considered in this thesis as the best for mimicking trabecular bone and reinforcing bone ingrowth is between 300 μm and 1200 μm. Similar arguments are the reasoning for the porosity value selection: 50-90% is the approximate trabecular bone porosity, however, around 50-80% porosity is wanted in this work due to >90% porosities could compromise the mechanical and structural integrity of the implant before degradation and substitution by newly formed bone [33].

Van Bael et al. [178] designed six distinct unit cells in three different pore shapes (triangular, hexagonal, and rectangular) to obtain the best pores geometry for a bone scaffold. The results showed a circular cell growth pattern which was independent of the pore shape. Therefore, pore size but not pore shape was found to significantly influence cells behavior. Nevertheless, Nguyen [44] performed a finite element analysis with square shapes and found stress concentration at the corners of the pores. Therefore, ellipsoidal shape was chosen to avoid corners and stress concentration in the implant pores.

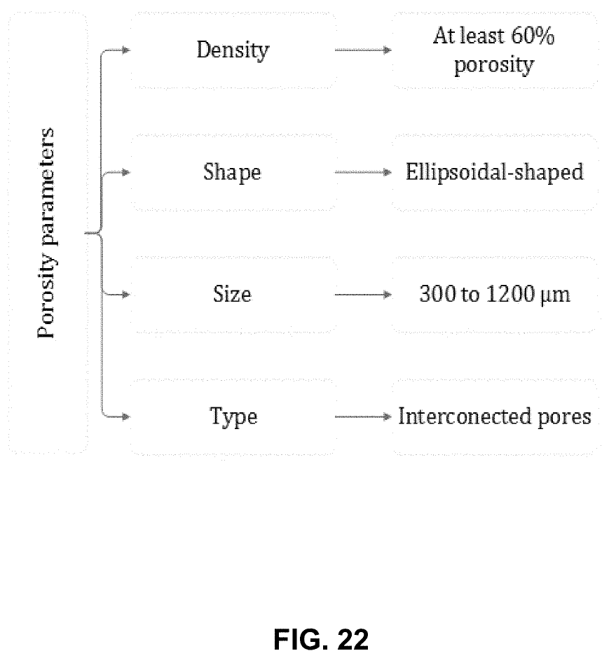

The architectural bone scaffold parameters determine the biological outcome [103], [178]. Hence, the fabrication route and characterization of a foam for osteoconduction that mimics bone morphology and structure are described. The selected parameters are shown in FIG. 22. It was necessary to adapt a method for optimizing the casting process described herein. The results are expected to provide useful information for further studies on the development of Mg-based foams and their applications in the field of bone implant.

### Experimental Methods

### Fabrication of Mg-Based Foams

The Mg-based foams fabrication assays were performed in Universidad Pontificia Bolivariana, Laboratorio de sintesis y procesos especiales, Medellin, Colombia. The method for obtaining the Mg-based foams was infiltration casting because of its replication capability. The starting material was an ingot of commercially available AZ31 Mg-based alloy. Then, foams were manufactured using a preform of salt particles sieved and separated in two average sizes, 500 μm and 1200 μm.

NaCl particles were dried and deposited into a refractory and the metal pieces were placed over the salt preform. Mg was protected from oxidation by using salt fluxes. At that time the crucible was sealed and the temperature was slowly increased from room temperature to around 680° C. Then, it was maintained during the whole process. No protective atmosphere was used.

As soon as Mg was completely molten, a vacuum was applied to create a negative pressure inside the chamber and this pressure was held for a short period. During this step, the liquid Mg was stirred occasionally to remove any corrosion layer that usually interfered with the melting process. Then, the system was cooled down at room temperature until the solidification of Mg/NaCl composite ended. The structure obtained was cut in several cylindrical samples that were leached by dissolution of the salt in water in order to reveal the open cells and the interconnected network of metal matrixes.

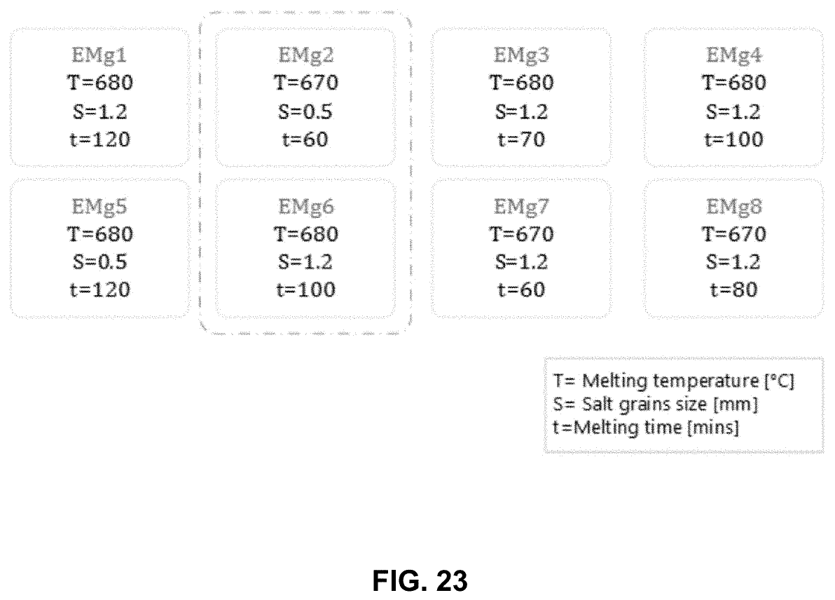

A total of eight assays were performed in order to assess control over the fluidity of the molten alloy. For the purpose of discussion EMg1-EMg8 nomenclature will be used. The process variables expected as metal fluidity and pore structure definers (melting temperature, salt grains size, and melting time), were mixed to have the best combination of the process variables. The values used in the eight different assays are shown in FIG. 23.

### Morphology Evaluation

The metal foam samples in this study were initially evaluated by stereo-microscope and pores of 500 μm and 1100 μm were estimated. Because of the small pore size a fairly high spatial resolution was required, leading to the use of X-ray μCT. The CT software and MIMICS software were then used to create 3D volumetric renderings by reconstructing the CT slices (radiographs). The X-ray power was set to the maximum of 8 Watts and 60 keV. This experiments were performed at the University of Illinois-Urbana Champaign, Beckman Institute for Advanced Science and Technology, Champaign, Ill., USA.

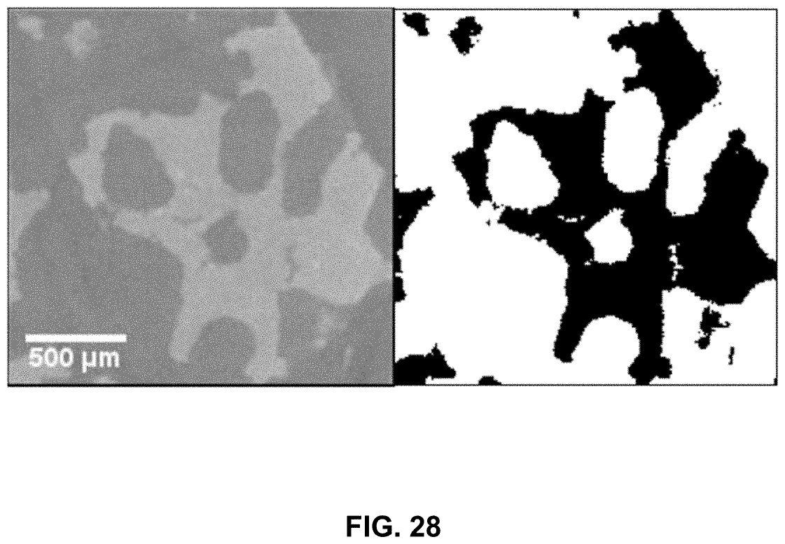

The porosity percentage in gray scale was estimated from X-ray μCT slides by converting the original slide to a binary image and obtaining a numerical calculation of the area covered by pores vs. the area occupied by material. ImageJ software was used for this purpose (FIG. 28). This analysis was repeated three times for each image and 10 slides were evaluated in order to ensure accurate data.

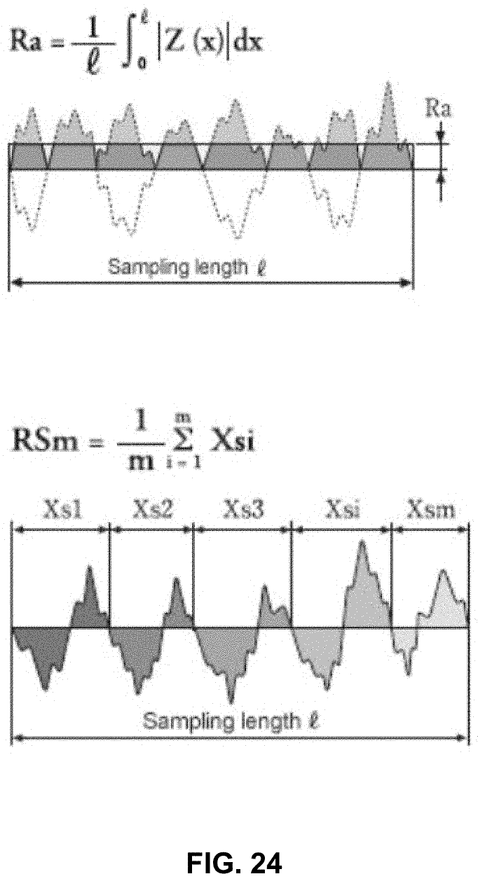

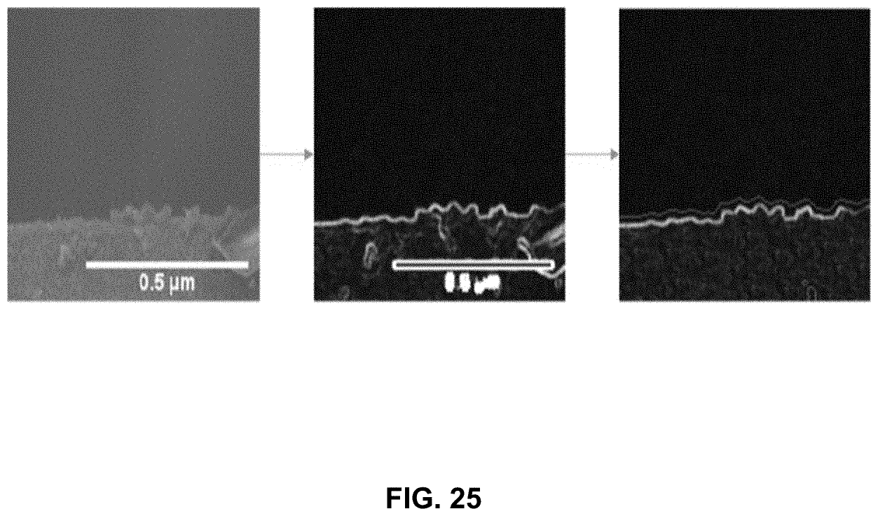

The surface roughness was evaluated and the approximate roughness was determined based on the profile lines of the surfaces on the porous structures in the 2D cross-sectional SEM images. These profile lines were obtained using an in-house developed MatLab tool, and were then used to calculate Ra and Sm roughness parameters [179], as shown in FIG. 24. In FIG. 25 the plot in the right displays the process of the profile line extraction.

Irregular samples were cut directly from two Mg-based foams. For the purpose of discussion, the following nomenclature will be used:

EMg6a-EMg6d: Mg-based foam with 500 μm pore size.
EMg2a-EMg2d: Mg-based foam with 1000 μm pore size.

The chemical composition was assessed by EPA test methods for evaluating solid waste with the techniques listed in Table 16 and Table 17, in Universidad Pontificia Bolivariana, Laboratorio Ambiental, Medellin, Colombia.

The crystalline phase of the samples was studied by X-ray diffraction (XRD) with a Cu Kα source at the University of Illinois-Urbana Champaign, Materials Research Laboratory, Champaign, Ill., USA.

### Compression Test

The density of the foam was calculated based on the weight and the dimension, according to Equation 5, where M represents the mass of the foam and V represents de volume by approximating the foam to a cylinder.

$$ρ = \frac{M}{V} \qquad ( Eqn 5 )$$

Uniaxial compression tests were carried out in a universal testing machine at a crosshead speed of 2 mm/min. The specimens for the compression test were cut to 2.5 mm in diameter and 2 mm in height.

### Results

### Infiltration Casting

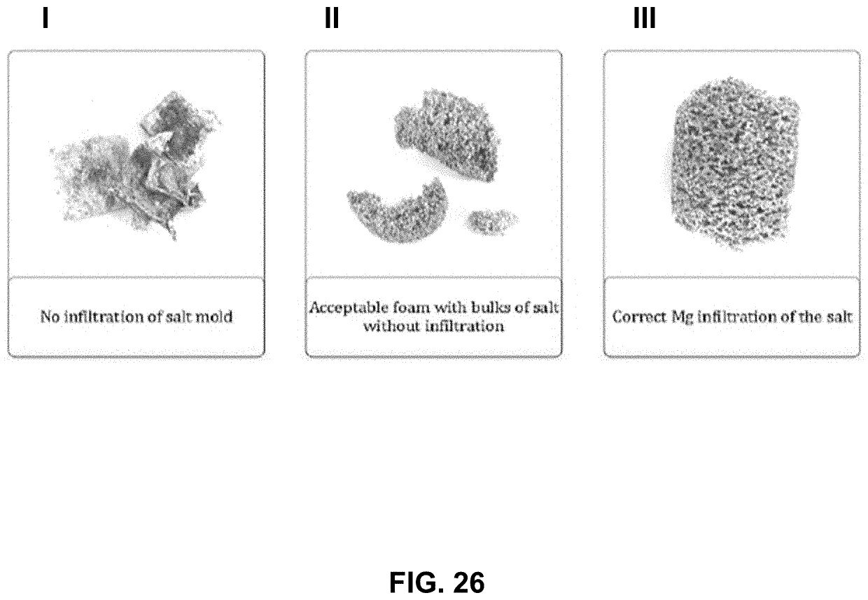

In the terms of the alloy's fluidity and the alloy ability to infiltrate the mould, three different behaviors were observed among the obtained foams (FIG. 26): I) no infiltration into the salt mold, II) acceptable foam with bulks of salt without infiltration, II) correct Mg infiltration into the salt.

The manufacturing parameters are shown in FIG. 23. There, the combinations of the variables that yield foams for bone replacement were marked by a dotted line. According to this, the most suitable parameters are a melting temperature of 670° C. and melting time of 60 mins for infiltrating 500 μm salt preform, and 680° C. and 100 min for infiltrating 1200 μm salt preform. Since that, EMg2 and EMg6 were selected as the sample foams for the remaining parts of this thesis.

### Morphology Evaluation

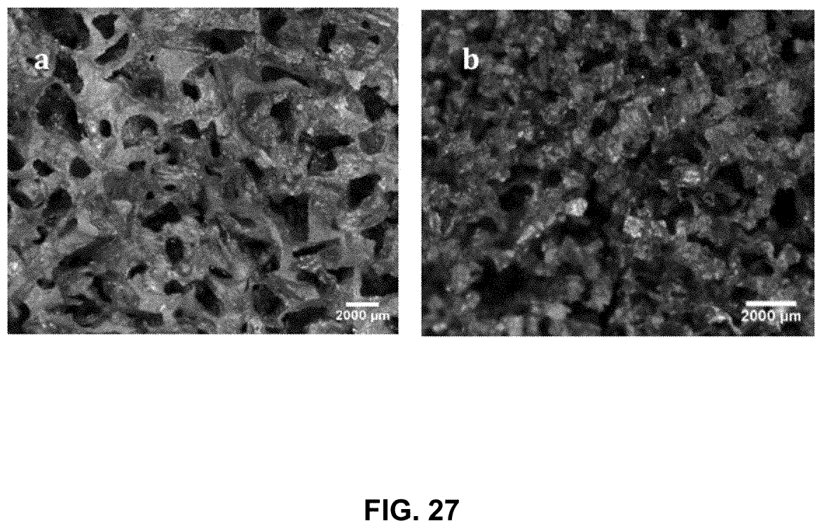

Corresponding stereo micrographs of the obtained open cell Mg foams are shown in FIG. 27. Pores of 500 μm and 1100 μm were initially estimated by this technique. In these images, it can be observed that both samples show open cell structures with interconnected pores.

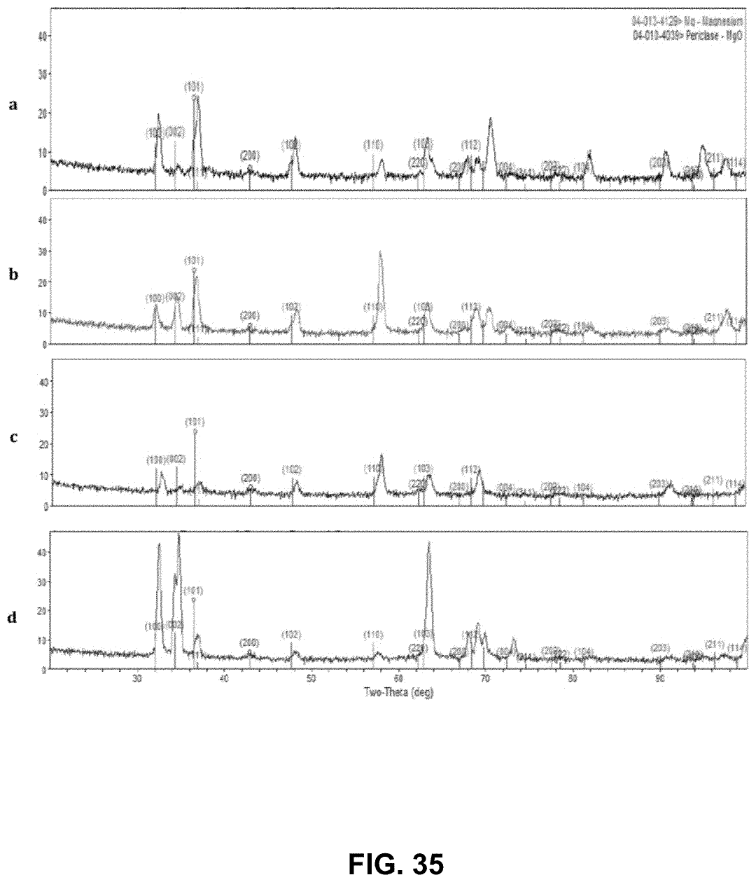

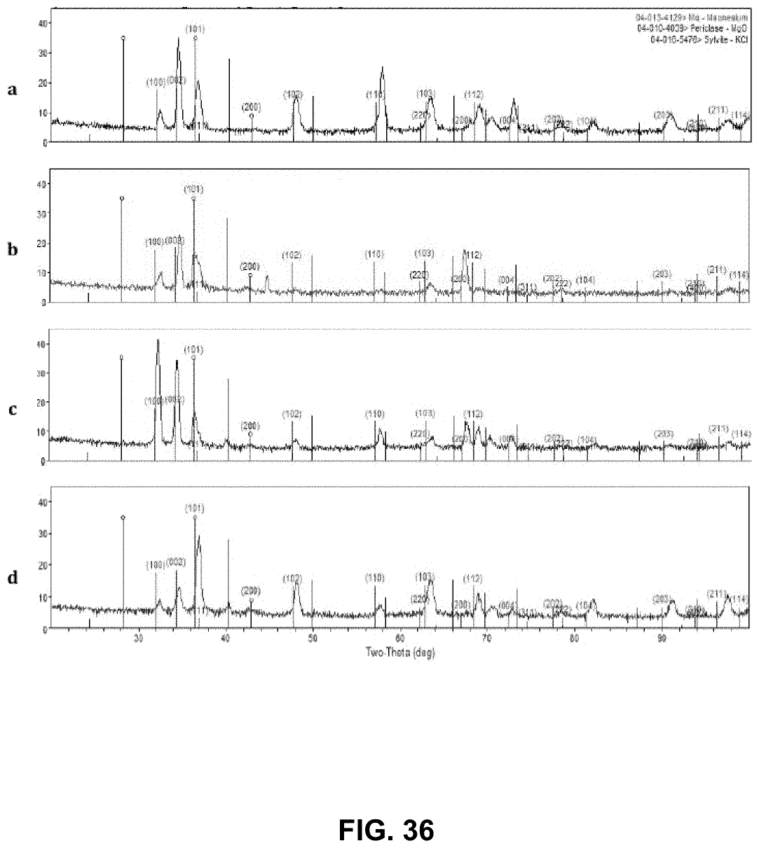

X-ray μCT provides an overview of the internal structure instead of only the surface as shown in the SEM images. Since the metal foam samples were Mg-based alloy, the images obtained presented high contrast between the metal (1.77 g/cm³) and the air (0.00119 g/cm³). FIG. 28 shows a 2D slice from X-ray μCT in the dimension scale of 500 μm. The dark gray sections are pores, while the bright gray represents solid, most of which is Mg according to the X-ray diffraction (XRD) analysis (FIG. 35 and FIG. 36).

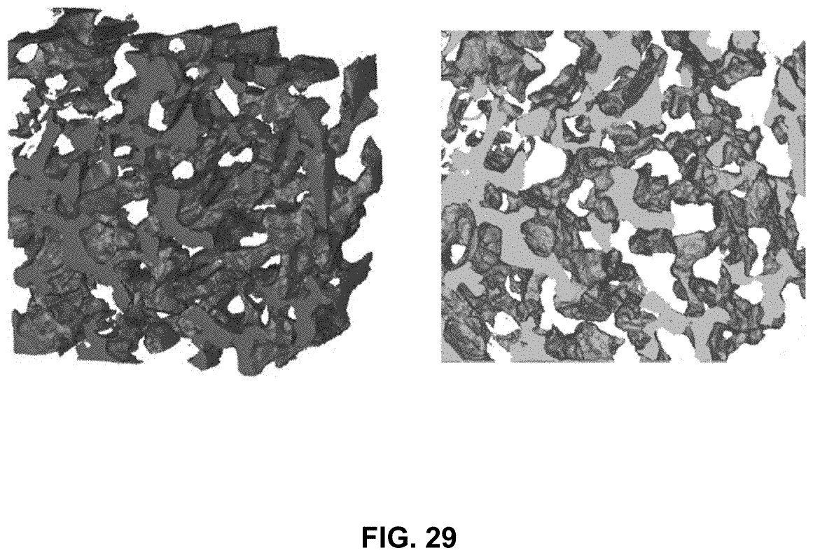

The boundary of the pores is more linear than curvy, and roughness values were obtained as Sm=27.16 μm and Ra=14.5 μm for EMg2, and Sm=13.36 μm and Ra=14.5 μm for EMg6, which are characteristic of the shape of salt preform and also agree with assumed ellipsoidal shape in the pores. The intraparticle pores, with diameters of less than 500 μm, were developed and most of them are interconnected as shown in 3D representation, FIG. 29. The volume data is processed to study in detail the interior structure. A representative volume with physical dimensions of 4.21 mm×4.82 mm×0.212 mm, was selected from the volume data (FIG. 29).

In CT practice, the proper grayscale value for defining a boundary between two phases is the average of their two mean end-member grayscales [134]. Therefore, the threshold gray scale was set as 150 for Mg and MgO area. With this threshold, binary images based on the segmentation of the pores were applied to estimate the porosity (FIG. 28). From FIG. 29 it can be seen that the highest porosity contribution for this material is in elongate pores followed by intermediate pores. Average porosities were determined as 87.19% for EMg2, and 71.35% for EMg6, using ImageJ.

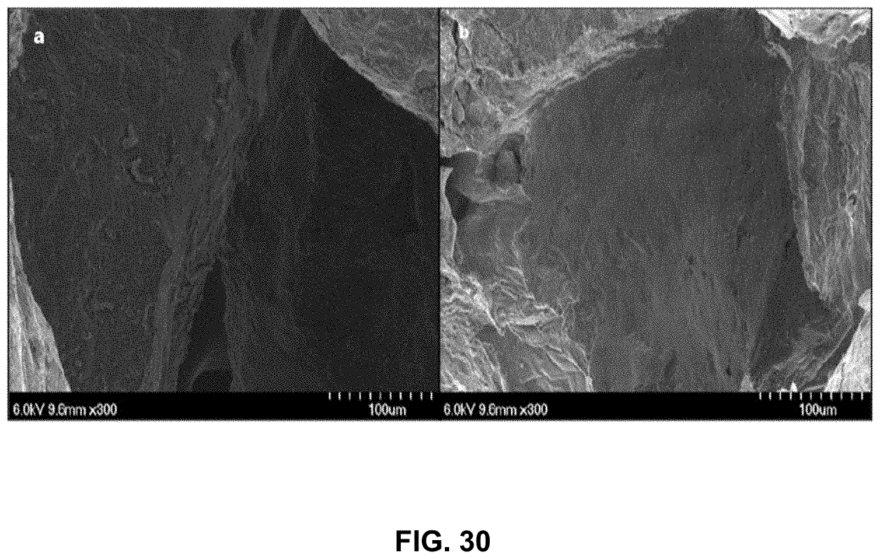

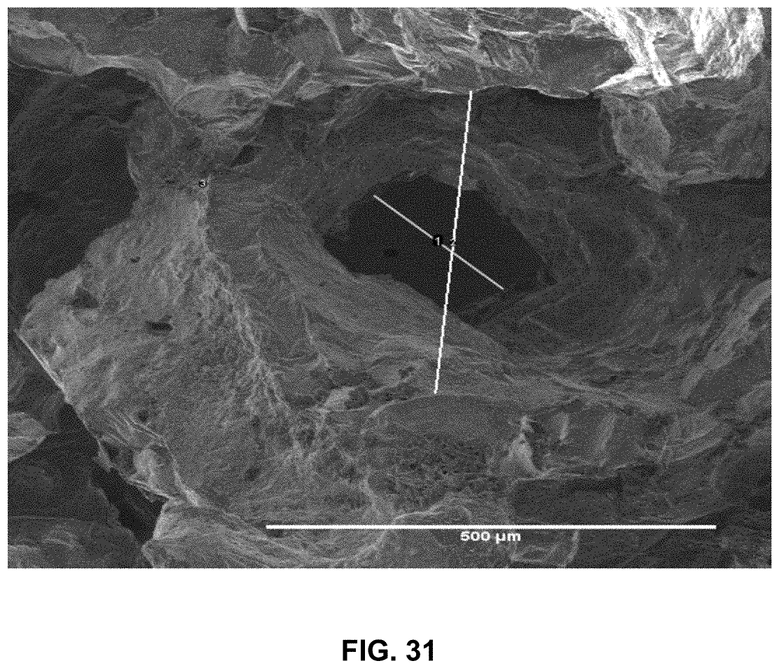

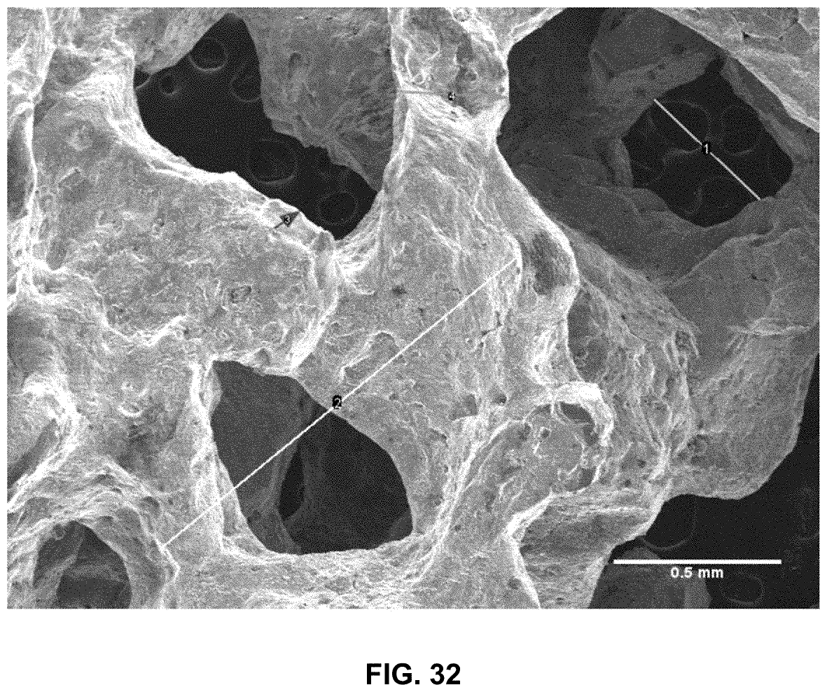

The two foam samples fabricated from the same ingot have similar porosity structures with different pore size, showing nearly isotropic characteristics in the surface, FIG. 30. The foams were discovered to contain mainly two types of pores (FIGS. 31 and 32): the macropores (white line) acquired as a result of the dissolution of salt grains and the small pores (cyan line) derivate from the interparticle contacts. The small pores (˜210 μm for EMg6 and ˜435 μm for EMg2) are usually distributed on the cell center, generating the connection tunnels that made the foams present very high open porosity (71.35% and 87.19%). The cell wall thickness (red arrow) was in the range of 90 μm and the cell edge (green row) has non-uniformity in dimension.

The size of the macropores, ˜497 μm for EMg6 and ˜1300 μm for EMg2, was mainly determined by the infiltration pressure, the salt particle dimensions and the casting temperature when the density of Mg is constant. Thus, it could be deduced that the infiltration parameters play a role on the connectivity of Mg foams, which would also provide great flexibility for the structural design of ellipsoidal pores Mg-based foams.

### Chemical Evaluation

EPA test methods for evaluating solid waste were used to investigate the levels of Al, Ca, Mg, K, and Zn extracted from EMg2 and EMg6. A summary of the applied methods and the results obtained is shown in Table 16 and Table 17.

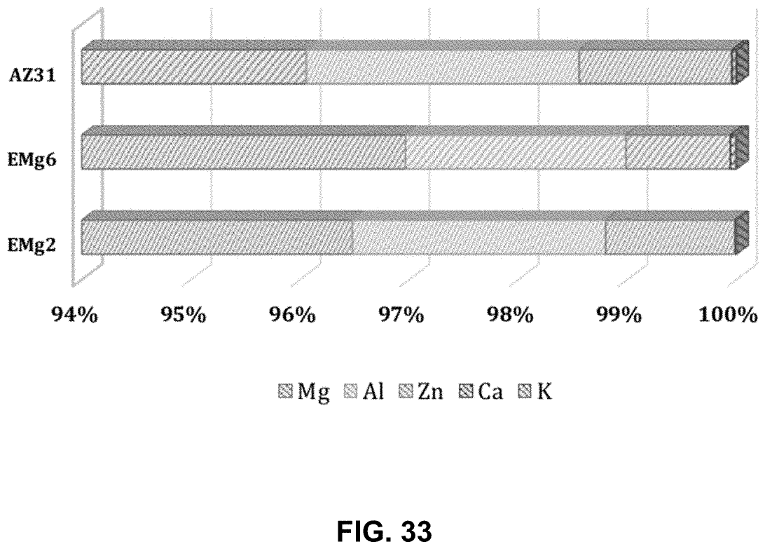

In both cases, the levels for all metals were hierarchized as follows: Mg>>Al>Zn>>Ca>K. The metal concentration levels in EMg2 and EMg6 are shown in FIG. 33. The results reveal only minor discrepancies between porous samples composition and AZ31 alloy composition [181], except for the apparition of a small K trace in the EMg2 and EMg6 samples.

TABLE 16
EPA test methods for EMg2

| Analysis | Method | Units | Results | Uncertainty |
|---|---|---|---|---|
| Al-Residue | EPA-1620 | mg Al/Kg_{(BS)} | 20934.015 | 372.629 |
| | EPA-3050 B | | | |
| | EPA-7020 | | | |
| Ca-Residue | EPA-1620 | mg Ca/Kg_{(BS)} | 100.443 | 2.009 |
| | EPA-3050 B | | | |
| | EPA-7140 | | | |
| Mg-Residue | EPA-1620 | mg Mg/Kg_{(BS)} | 870284.103 | 17405.682 |
| | EPA-3050 B | | | |
| | EPA-7450 | | | |
| K-Residue | EPA-1620 | mg K/Kg_{(BS)} | 4.930 | 0.099 |
| | EPA-3050 B | | | |
| | EPA-7610 | | | |
| Zn-Residue | EPA-1620 | mg Zn/Kg_{(BS)} | 10718.711 | 214.374 |
| | EPA-3050 B | | | |
| | EPA-7950 | | | |

TABLE 17
EPA test methods for EMg6

| Analysis | Method | Units | Results | Uncertainty |
|---|---|---|---|---|
| Al-Residue | EPA-1620 | mg Al/Kg_{(BS)} | 16535.377 | 294.333 |
| | EPA-3050 B | | | |
| | EPA-7020 | | | |
| Ca-Residue | EPA-1620 | mg Ca/Kg_{(BS)} | 432.579 | 8.652 |
| | EPA-3050 B | | | |
| | EPA-7140 | | | |
| Mg-Residue | EPA-1620 | mg Mg/Kg_{(BS)} | 794641.757 | 15892.835 |
| | EPA-3050 B | | | |
| | EPA-7450 | | | |
| K-Residue | EPA-1620 | mg K/Kg_{(BS)} | 37.890 | 0.758 |
| | EPA-3050 B | | | |
| | EPA-7610 | | | |
| Zn-Residue | EPA-1620 | mg Zn/Kg_{(BS)} | 7832.828 | 156.657 |
| | EPA-3050 B | | | |
| | EPA-7950 | | | |

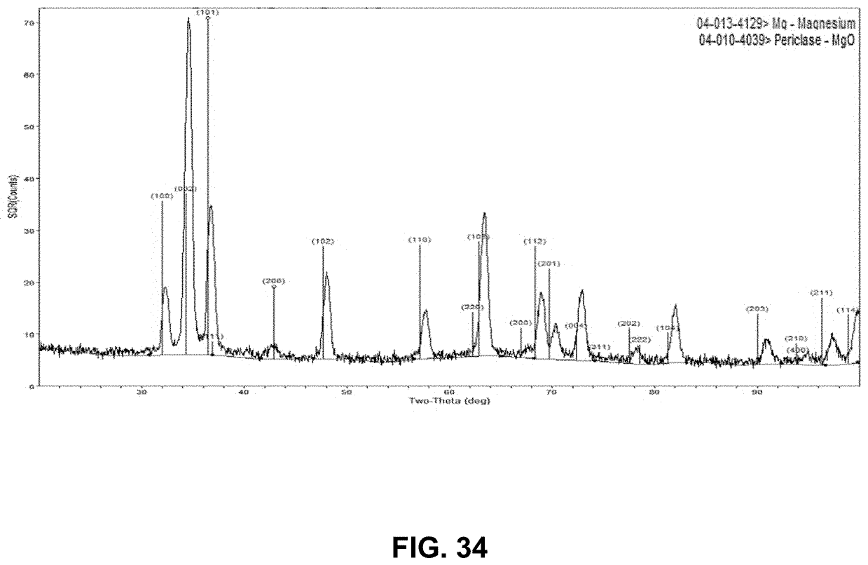

The crystalline phase of surfaces and original material was studied by XRD. FIG. 34 shows the XRD patterns of the original ingot. This material contains mainly two kinds of picks: Mg and MgO. No other phases were identified within the sensitivity limits of XRD. Zn and Al elements might not be detected because only small amounts of them are present on the alloy.

The peaks that appeared at 2e=42.9° and 78.4° respectively, represent (2 0 0) and (2 2 2) planes of the cubic MgO crystal structure.

FIG. 35 shows that the phase compositions of the Mg-based foams are very similar to the original ingot, and same peaks of MgO can be identified. FIG. 36 shows a comparable XRD pattern. However, MgO peaks were almost completely disappeared in samples EMg6a and EMg6b (FIGS. 36A and 36B). There is also a peak at 2e=42.22° that turns up in samples EMg6c and EMg6d (FIGS. 36C and 36D), this peaks represent KCl, which was one of the salt fluxes used during the foams infiltration process.

In all the cases, original ingot and obtained foams, Mg peaks were shifted to lower 2e values which could correspond to the dissolution of the larger Al and Zn atoms into the Mg matrix. Sometimes, doping introduces a low amount of impurities or more generally point defects in a crystal [182].

### Compression Test

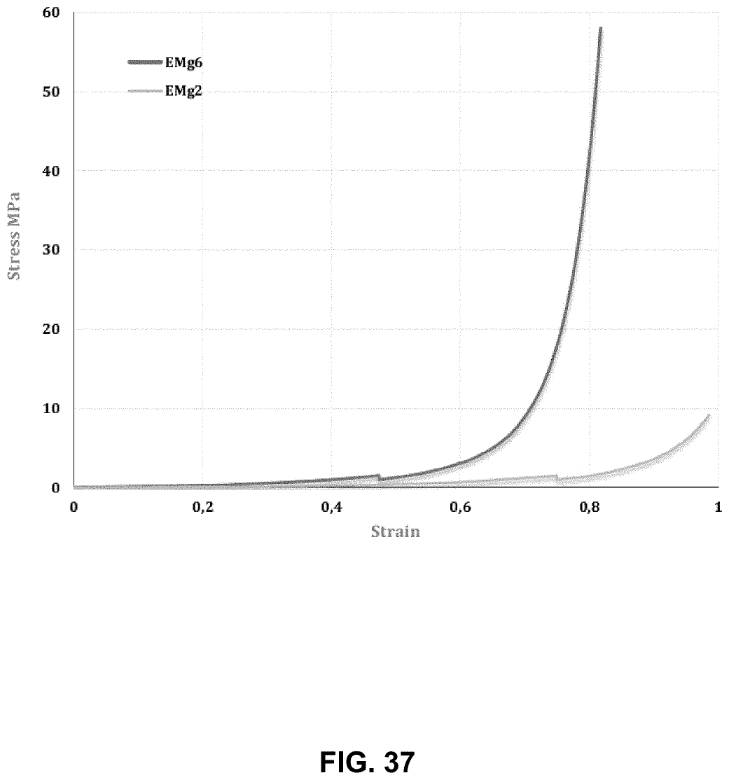

The effects of density and pore size on the mechanical properties of the Mg-based foams were examined by compressive test, as shown in FIG. 37. The curves exhibit a quite long plateau with a nearly constant stress level where the stress increases slowly as the cells deform plastically. This region ends at strain as high as 65-75% for EMg6 and 80-85% for EMg2. Then, there is a densification stage where the stress rapidly increases while the collapsed cells are compacted together.

The plateau regions are smooth, suggesting that foams are not brittle. This smooth plateau reflects the compaction deformation mode. As the compression goes on, the foam compacts quickly, therefore, it can be seen a point at 48% strain for EMg6 and 75% for EMg2 were the foams are compacted and a change in the foams stiffness is noticeable. It can be seen that there is an inverse relationship between the achieved porosity in the scaffolds and their mechanical response; the higher the porosity the lower the mechanical properties found.

### Discussion

Emerging casting technologies for Mg alloys were inspired by the Al foundry techniques. Al casting processes were already developed, and successfully optimized [116], [117]. However, Mg-based alloys have a lower density and volume heat capacity compared to Al-based alloys. Sintering of Mg-based alloys is challenging because of the high affinity of Mg. Therefore, casting fillings are inherently difficult due to low metallostatic pressure and rapid alloy solidification [183]. Fluidity was considered a crucial casting property because it defines the ability of metal to fill the mold cavities. Therefore, this property may be improved by manipulating the casting parameters.

Fluidity was one of the important steps in the manufacturing process since a high fluidity level or an extended holding time would result in an over-infiltrated NaCl template. Yim et al. [184] found an increase of fluidity of AZ31 with an increase in temperature and melting time. Moreover, oxide films on the melt surface can significantly raise the surface tension and viscosity of the molten metal and reduce its ability to fill the mold [22], [183], [184]. Mechanical destruction was used to break down the oxide layers to achieve the desired fluidity values to fill the salt cavities. Consequently, in this thesis a combination of high temperature and large melting time, as in the case of EMg5 FIG. 23, resulted in an over-infiltrated foam. While a combination of low temperature and low melting time, as in the case of EMg7, FIG. 23, resulted in the apparition of the oxide layer and a noninfiltrated foam.

The versatility provided by this technique allowed the fabrication of Mg-based implants with two different pore sizes which can mimic the complex architecture of bone to optimize bone tissue regeneration. The more suitable process parameters mainly depend on the alloy interaction with the salt. It can be seen in FIGS. 31 and 32 that pores are rounded with a non-uniform and non-homogeneous distribution, and centered largely in ˜497 μm for EMg6 and ˜1300 μm for EMg2. The foams inherited the shape and the cell sizes of the salt particles indicating that the morphology and sizes of the pores can be controlled by selecting an appropriate salt preform. Furthermore, the internal surfaces of the pores are smooth and reflect the surface quality of salt particles (FIG. 30).

The cover fluxes melted over the surface of the molten alloy formed a protective coating which inhibits the molten metal from contacting ambient gases. Moreover, salt fluxes are suspected to attract and wet impurities in the Mg melt, resulting in their subtraction [183]. Therefore, although there was no protective atmosphere, chemical analysis suggests that no chemical reaction occurred during the infiltration process, showing a very promising application prospect as biodegradable bone implant material. The amount of MgO observed on the pore wall, was probably due to the surface oxidation of the foams stored at room temperature and also oxidation occurred during salt grains dissolution.

AFM is a measuring technique that can be applied to scan large areas for micro-scale roughness of surfaces with a small curvature [185]. For our highly porous materials with small contact area inside de porous, AFM can no longer measure the surface. Hence, the surface and pore morphology were observed on a scanning electron microscopy (SEM). Very smooth surfaces lead to images with uniform brightness levels, whereas rough surfaces have a variety of gray values. With this understanding, Banerjee et al. recognized that analysis of the gray scale of the SEM images could represent a noncontact surface indicator [186].

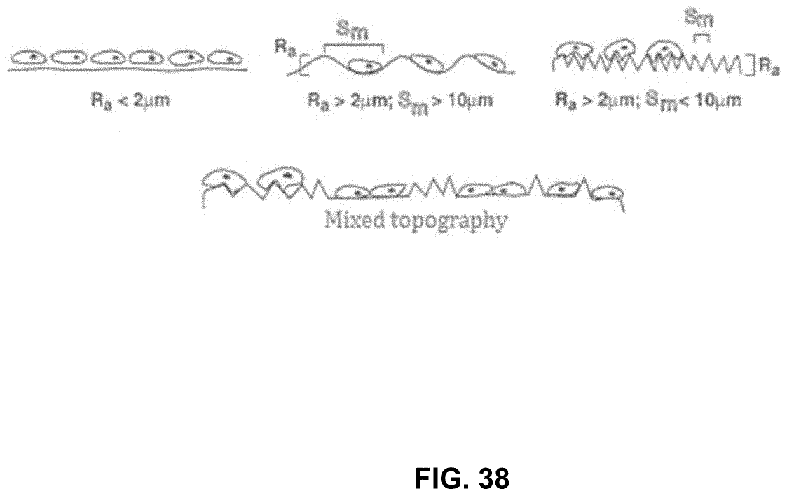

The real surface topography is so complicated that a finite number of parameters cannot provide a full description. Although, an amplitude parameter (Ra) and a spacing parameter (Sm) [179] were selected in order to have a better comparison with other authors' results. Anselme and Bigerelle [187] evaluated the influence of the surface topography on cell adhesion, their results agree with the consensus of Badenas [38], who attempted to determine a threshold in sensitivity on human adherent cells, which is shown in FIG. 38.

They concluded that the cells need to perceive the microroughness for answering the stimulus. Therefore, roughness values may be in the order of the cells size. The roughness values obtained in this thesis were Sm=27.16 μm and Ra=14.52 μm for EMg2, and Sm=13.36 μm and Ra=6 μm for EMg6. These values could correspond to the second behavior of the consensus shown in FIG. 38, when Ra>2 μm and Sm>10 μm and Sm< to the size of an osteoblast which is reported to be 20-30 μm [188]. According to that, this surface could be considered by the cells as a smooth surface, and cells can be attached by focal contacts and topography peaks. Thus, it could force the cells to assume a more osteoblast-like form.

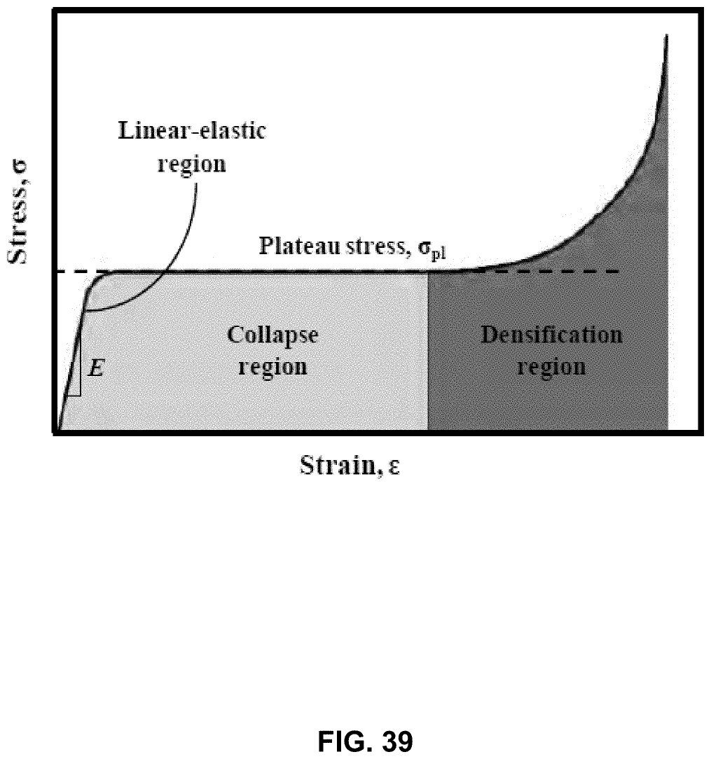

The typical compressive stress-strain curve for metal scaffold with high porosity shows three differentiated behaviors (FIG. 39) [189],[190]: Firstly, there is a linear-elastic region which is characterized by an initial increase in stress. This initial high slope is associated with the stiffness of the porous samples. Subsequently, due to the collapse of the pores, the flow stress no longer increases with strain and there appears a wide stress plateau known as plateau or collapse region.

The compression curves obtained in this thesis began with the plateau region. These regions are smooth, without the presence of serrations which are typically observed in other open-cell Mg-based foams [23], [25]. This result suggests that foams are not brittle. The increase of plateau stresses is very slow and the densification did not begin until about a strain of 65% or over was reached. This means that the present Mg-based foam could be good in impact-absorption applications.

### Conclusions

Described herein is the fabrication and characterization of Mg-based foams by infiltration casting suitable for manufacturing Mg-based foams for bone tissue engineering. Foams with two different porosities and pore sizes were obtained. For the infiltration process temperature and melting time have been defined as being 680° C. and 670° C. and 100 mins and 60 mins for infiltrating 1200 μm and 500 μm salt preform respectively. The precise selection depends on the pore size and porosity desired.

The result of the crystalline phase and chemical analysis further indicate a safe and biologically inert process. This avoids the problem of toxic solvents or materials and hold significant promise for biomedical application as bone interfacing implants. The foams also presented enough mechanical properties to fulfill the required mechanical response of some scaffold materials, depending on the porosities. The observed topography can be considered an advantage from a bone-implant interface point of view as potential surface for cell adhesion, osteoblastic differentiation, and osteoconduction. Surface modification of biodegradable porous Mg-based implant

### Introduction

The first step in the process of designing a surface for a biological process is to define two important terms: roughness in this thesis refers to patterns and structures that appear from the process of manufacturing a material and to mechanical characteristics exhibiting randomness and polydispersity in terms of size, shape and periodicity, as the roughness values described herein. While the term designed surface will be used in topographies with a specific target.

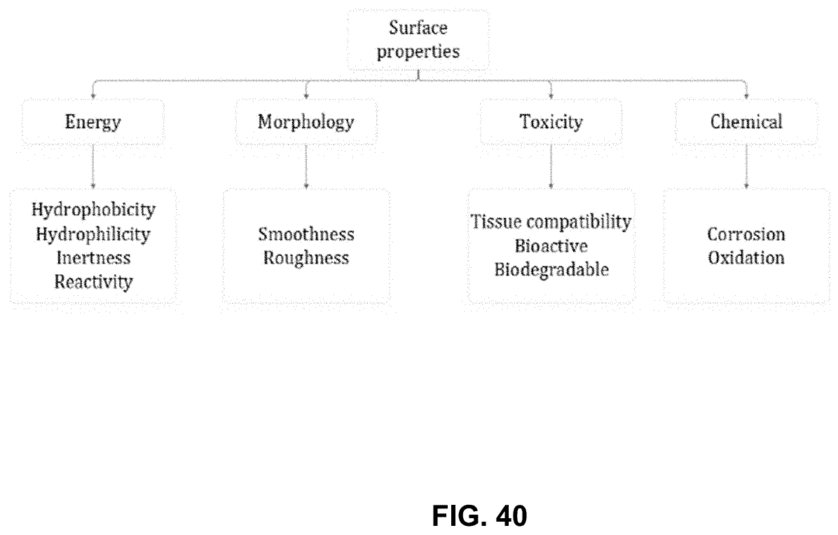

In the case of designed surfaces, some authors have selected characteristics taking into account a specific biological response, such as cell morphology, cytoskeleton organization, gene expression, proliferation, and adhesion [192]-[194]. Once a particular type of cell, organism or biological system is selected, this topography becomes a predefined system in which the geometry, size and arrangement of features are adapted to elicit a specific response in the living target. When a surface is designed for influencing a natural process the surface is called bio-inspired design[195]. In this work, the bio-inspired surface does not pretend to be a structural and dimensional copy of a natural form, but a design adaptable to intentionally induce a biological response. The characteristics that define the activity of a surface are listed in FIG. 40.

It is also important to introduce and identify the scope of the terms attachment and adhesion. The first one in the English literature refers to the number of cells that bind to the surface in the first hours. While the second one is used to incorporate the idea of the strength of the bond between cells and substrate [38]. In this example, the terms adhesion or attachment will be used as the measure of the number of cells adhered to the surface.

Mg-bone and bone tissue interaction is dynamic (i.e. bio-resorbable), as opposed to that of Ti alloys and stainless steels devices. Nevertheless, Mg and its alloys are highly reactive materials and exhibit extremely low corrosion resistance in surroundings containing chloride; that restricts their applications especially in the physiological environment and obstructs their extensive use in orthopedic applications [196], [197]. The corrosion dynamics is certainly complex and it has been established that the degradation rate is too large, particularly in the early stage [124]. As a result of their poor anticorrosive properties, Mg alloy implants cannot enhance the cell response to bone formation around the implant or successfully integrate with the host tissue, mainly at the initial implantation [16]. Consequently, in order to have enough interaction between Mg implant and tissue, Mg-based foams should be designed in a way to control the surface degradation.

Furthermore, Mg dissolution results in emission of hydrogen and basification, which means, an increased pH in the implantation zone. When the OH— concentration neighboring the surface increases to a certain extent, Mg or Ca containing phosphates precipitate and form an external layer on the surface [124], [198]. Hence, the corrosion process will alter the interface between the Mg-based biomaterials and the environment. Commonly, the surface morphology, topology, and composition, which play roles in the efficacy of artificial implants, can alter protein absorption which mediates adhesion of cells [195], [199]. The interactions between biological environments and biomaterials take place on the material surface, and the biological response from living tissues to these biomaterials depends on the surface characteristics [200].

Significant effort has been applied to developing methods for preventing fast Mg degradation in vitro and in vivo [139], [152], [201]. Addition of alloying elements is one of the first approaches. For instance, Zn improves the alloy mechanical conditions due to solid solution strengthening. It can also improve the castability but in larger amounts (>2 wt. %) Zn leads to an embrittlement of the material [19], [65]. Descriptions of the effects of alloy elements on the Mg corrosion behavior are listed in Table 7.

Since alloying has not proven to be sufficient to overcome the issues stated above [19], [202], surface modification of Mg by chemical, and physical means, and a combination of both has been used for treating it. Wu et al. [124], wrote a review describing surface design and potential methods for surface modification of Mg alloys. Since then, techniques such as electrochemical deposition [203]-[205], plasma electrolytic oxidation [206], [207], non-substrate-involving coatings as physical vapor deposition[208]-[210], ion implantation [211]-[213], and polymer [92], [214] and composite-based coatings [215]-[217] have been investigated.

However, in the case of Mg-based foams, the purpose of the surface design is to alter but not to change the overall bulk material structure and properties. Moreover, the surface thickness and density factors influence the anticorrosion performance [218]. Nevertheless, the benefit obtained with the lightweight properties of Mg-based foams could be lost if the surface is modified and increased in its thickness and density. Thus, it is important to develop a modification method to improve the corrosion resistance, while maintaining the lightweight properties of Mg-based foams. Surface modification may provide a means to selectively enhance anticorrosion property and cellular response without affecting the required bulk attributes of Mg and its alloys. In this respect, surface modification to form a hard, biocompatible and corrosion resistant modified substrate has recently become a focus for research and development of Mg-based biomaterials [124], [219].

Recently, various methods have been explored to engineer hydrophobic surfaces on Mg-based materials [220]-[224]. Generally, the way to fabricate a hydrophobic surface implicates two steps: roughening a surface and lowering its energy. In the current state of this review, only one modification technique to improve corrosion resistance of porous Mg-based alloys on biomimetic calcium phosphate coating has been found [44]. As a result, it did not perform as good as when applying roughness on Mg surfaces. Meanwhile, surface roughness has an effect on adhesion, proliferation, differentiation, and protein synthesis of osteoblast cells [225].

Hydrophobic surfaces can prevent contact of Mg-based materials with water in order to reduce the prolongation of corrosion, especially in a humid environment [226]. An increase in proliferation and adhesion, with an increase in surface roughness, can occur as well [195], [227]-[229]. Therefore, the fabrication of hydrophobic surfaces on the Mg-based foam is considered to improve its corrosion resistance. An effective method to decrease the progress of corrosion is to fabricate a water-repellent surface to prevent the infiltration of water into porous Mg-based substrate. A hydrophobic substrate would inhibit the contact between the surface and water and environmental humidity, while an increase in surface roughness can enhance adhesion and proliferation of cells. A modification route intended to create a hydrophobic surface modified by low-energy irradiation is described herein as a promising method to improve the performance of porous Mg-based materials corrosion, because it could inhibit the contact of the Mg surface with water, cell secretions and environmental humidity without changing definitely the bulk properties of Mg-based foams.

### Experimental Methods

### Samples Preparation

Irregular samples were cut directly from two Mg-based foams. For the purpose of discussion, the following nomenclature will be used:

EMg6: Mg-based foam with 500 μm pore size.
EMg2: Mg-based foam with 1000 μm pore size.

### Irradiation

Samples were exposed to a low-energy Ar+ irradiation with two different ion energy values. The energy values selection process is described below. Then, samples were stored and labeled with the ion energy conditions to which they were exposed. These conditions are shown in Table 18.

TABLE 18
Irradiation Conditions

| | Conditions | | | | |
|---|---|---|---|---|---|
| | Working pressure | Gas pressure | Energy | Current | Time |
| Samples | [Torr] | [Torr] | [eV] | [mA] | [mins] |
| EMg6a | 5 × 10⁻⁷ | 5 × 10⁻⁴ | 600 | 9 | 8 |
| EMg6b | 8.2 × 10⁻⁷ | 5.4 × 10⁻⁴ | 400 | 10 | 5 |
| EMg2a | 5 × 10⁻⁷ | 5 × 10⁻⁴ | 600 | 9 | 5 |
| EMg2b | 8.2 × 10⁻⁷ | 5.4 × 10⁻⁴ | 400 | 10 | 5 |

From XRD patterns EMg2a, EMg2c, EMg6a and EMg6b samples (FIGS. 35b, 36a, and 37c, respectively) were selected for exposition to a low-energy Ar+ irradiation. The ion energy values were identified from the literature [230]-[234] taking into account the incidence of low energy Ar+ irradiation in MgO surfaces roughness, considering the constant presence of MgO in the samples surface, and the roughness influence in corrosion and biological behavior of materials [235].

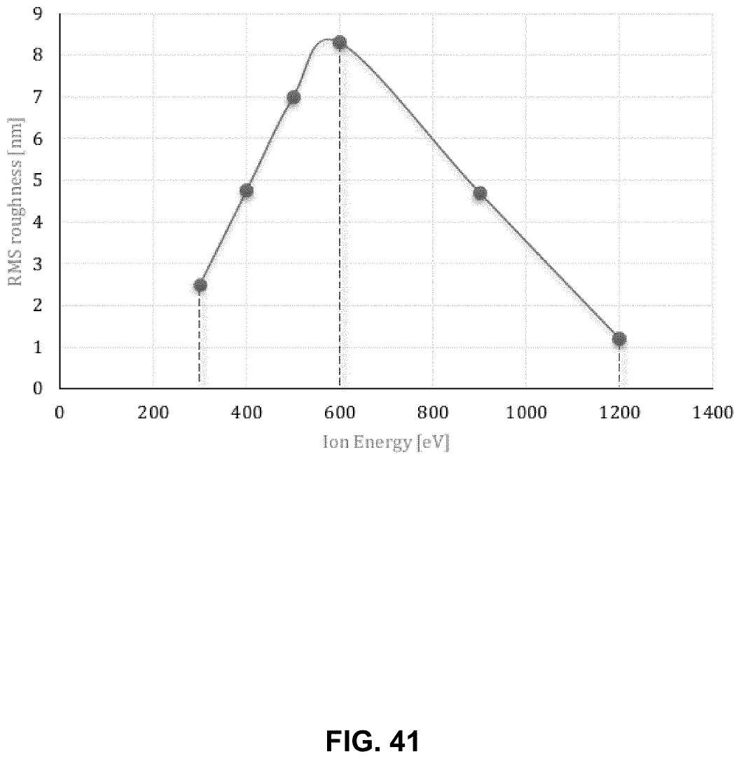

In the literature cases [230], [233], [236], the roughness root mean square (RMS) increases at first, reaches maximum and then, decreases in a relatively higher energy region. The RMS value reaches maximum at about 400 eV to 500 eV instead of increasing with the ion energy. It keeps increasing before 400 eV, decreases after 600 eV to 750 eV and later drops to almost 0. Hence, the roughness maximum will be found between the regions of 400 eV to 700 eV. A summary of the low energy irradiation effect on the MgO surface roughness is shown in FIG. 41.

Since MgO surfaces roughening capability showed to be dependent on ion energy in the region from 200 eV through 750 eV, the discussion of the authors [231], [233], [236], [237] about the variation of secondary electron emission coefficient (γ) was reviewed in order to understand the behavior of the surface morphology. Summarizing, the surface roughness is a relevant factor which intensely influenced they value, in (111)-oriented surfaces they increased as the surface became smooth. The crystal orientation strongly affects the γ value. Kim et al. [237] described that a (111)-oriented surface had a higher γ than films with (200) or (220)-orientation (the planes that emerged in XRD patterns, FIGS. 34-36). For (200)-oriented films with low γ, the γ value became highest when the ion energy was between 350 eV and 500 eV. At that moment, the γ decreased as the energy increased and surface became smooth [234]. Therefore, the ion energy values chosen for samples irradiation were 400 eV and 600 eV.

Once ion energy values were defined, foams were fixed on a target holder, and the ion beam was directed onto the foam. In these experiments, the ion energy was varied while the other parameters were kept as constant as possible. The irradiation conditions were summarized in Table 19. There was no control over the incidence angle because of the random porous structure.

### Corrosion Test

The irradiated and non-irradiated specimens were cut to the dimensions of approximately 7 mm×7 mm×10 mm to evaluate the corrosion rate by the immersion corrosion test method. The corrosion test specimens were cut from the top of samples exposed to guarantee that they were irradiated. Then, the specimens were washed with distilled water, 70% ethanol and dried by vacuum.

The predominant factors that have a greater influence on the corrosion behavior of Mg-based alloy were identified from the literature [238]-[241]. As the range of individual factor was wide, a two-factor, three-level factorial design matrix was selected with one replication and blocks between the replications to differentiate the samples by the pores size. This design implies 18 runs for a quantitative evaluation of the influence of ion energy (E) on biological and corrosion behavior of the Mg-based foams and how the effect of the E varies with the level of corrosive medium exposition time (T). Hence, the response variables were Weight Loss and pH. Table 19 represents the range of factors considered and Table 20 shows the 18 sets of values used to conduct the experiments. The method of designing such a matrix is dealt with elsewhere [242].

The 18 experiments were carried out in random order. The more freely experimental runs are randomized, the less extraneous factors possibly affect the results. Minitab statistical software, version 16, was used to analyze the significance of the effects of the factors via Analysis of Variance (ANOVA). The ANOVA was used to find the significant main and interaction factors. The significance of all terms in the linear relationship will be judged statistically by computing the F-value at a probability (P)<0.05.

TABLE 19
Corrosion test factors and their levels

| | | | | Levels | | |
|---|---|---|---|---|---|---|
| No | Factor | Notation | Unit | A | B | C |
| 1 | Energy | E | eV | 0 | 400 | 600 |
| 2 | Exposure | T | Hours | 24 | 48 | 96 |
| | time | | | | | |

TABLE 20
Corrosion test design matrix

| | | Blocks | | | Weight | |
|---|---|---|---|---|---|---|
| Standard | Run | (pores size) | T | Energy | Loss | |
| Run | order | [μm] | [h] | [eV] | [mg] | PH |
| 16 | 1 | 1000 | 96 | 0 | 2.5 | 6.5 |
| 15 | 2 | 1000 | 48 | 600 | 1.5 | 6.5 |
| 18 | 3 | 1000 | 96 | 600 | 1.6 | 6.5 |
| 10 | 4 | 1000 | 24 | 0 | 0.9 | 7 |
| 13 | 5 | 1000 | 48 | 0 | 1 | 6.5 |
| 11 | 6 | 1000 | 24 | 400 | 0.1 | 9 |
| 12 | 7 | 1000 | 24 | 600 | 0.6 | 9 |
| 14 | 8 | 1000 | 48 | 400 | 0.5 | 7 |
| 17 | 9 | 1000 | 96 | 400 | 0.3 | 6.5 |
| 7 | 10 | 500 | 96 | 0 | 2.7 | 6 |
| 5 | 11 | 500 | 48 | 400 | 0.2 | 7 |
| 4 | 12 | 500 | 48 | 0 | 1.6 | 6.5 |
| 6 | 13 | 500 | 48 | 600 | 1.5 | 6.5 |
| 2 | 14 | 500 | 24 | 400 | 0.6 | 8.5 |
| 8 | 15 | 500 | 96 | 400 | 0.5 | 7.5 |
| 1 | 16 | 500 | 24 | 0 | 1.9 | 8 |
| 3 | 17 | 500 | 24 | 600 | 0.6 | 8.5 |
| 9 | 18 | 500 | 96 | 600 | 1.8 | 7 |

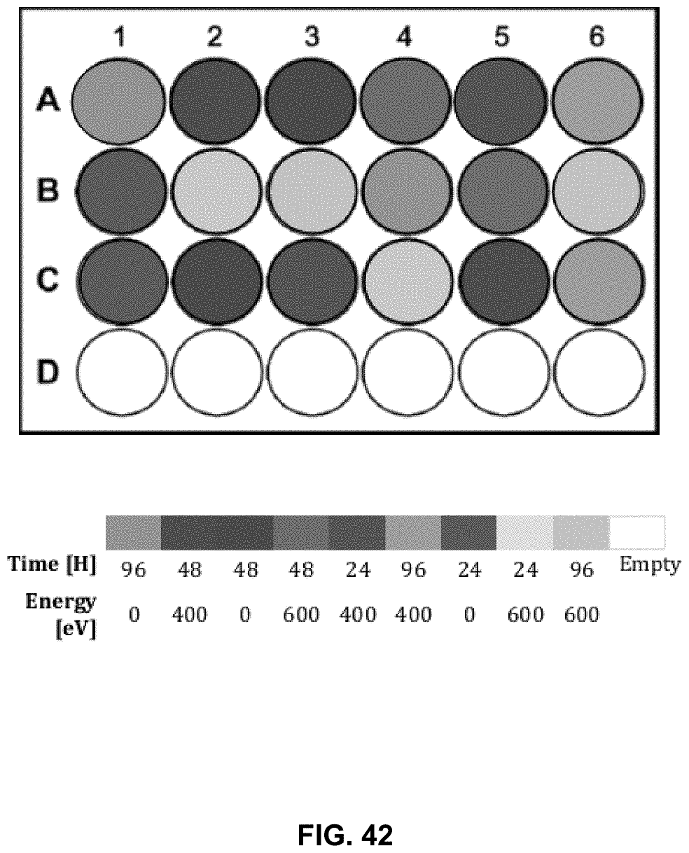

Solution of Medium 231 and SMGS, which has an ionic composition and concentration similar to that of human environment, was prepared. The pH value of the solution was maintained at pH9 with concentrated HCl. The test method consists of immersing the specimens in a 24-well flat-bottomed cell culture plate filled with culture Medium 231 and SMGS at 37° C. temperature and 5% CO₂. The distribution of the samples in the plate is shown in FIG. 42. The pH value was measured using pH Test Strips. Strips were immersed fully in the medium with the sample and left for a minimum of 3 minutes (and until there was no further color change) before reading.

The corrosion rate of the alloy specimen was estimated by weight loss measurement as described by [238]. The original weight (w0) of each sample was recorded and the specimen was then immersed in the solution. The immersed samples were taken out after 24 h, 48 h and 96 h. The morphology and composition of the corrosion products layer adhered to the surface were assessed by optical microscopy. For the testing, the corrosion products were removed by submerging the specimens for two minutes in hydrochloric acid. Finally, samples were gently rinsed with flowing distilled water and 70% ethanol for a few seconds, dried at room temperature to avoid cracking and weighed again to obtain the final weight (w1). The weight loss (w) was measured using the following relation w=(wo−w1).

### Viability Test

### Liquid Extract of the Material

The experiments were performed following ISO 10993-5:2009 [167] for biological evaluation of medical devices. Samples were sterilized by steam in an autoclave (3 cycles of 15 minutes at 121° C.). The extraction vehicle was culture Medium 231 with serum because of its ability to support cellular growth as well as extract both polar and non-polar substances [167]. The extraction was performed in a sterile, chemically inert and closed container. The conditions of the extraction were 24±2 h at 37±1° C. Thereafter, dilutions of the series of the extracts were made from the original extract by using Medium 231 as a diluent. Finally, 75%, 50%, 25% concentrated dilutions were obtained, and the Medium 231 without extract was used as a control.

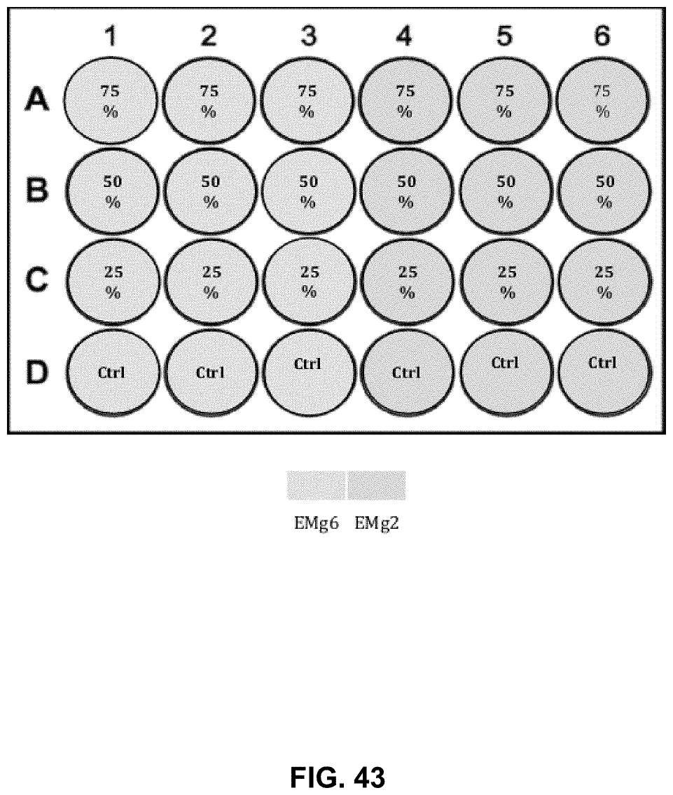

Cells were subcultured twice before using them in the Mg experiments. Then, they were seeded at 4×104 cells/cm², and grown for 48±2 h in air with CO₂ at 37±1° C. Each individual experiment was carried out in triplicate wells in a 24-well flat-bottomed cell culture plate, the distribution of the liquid extracts of material in the plate is shown in FIG. 43. After 48 h, cells were harvested and the number of viable cells was estimated by trypan blue exclusion test, as described by [243]. Cells were washed three times with phosphate buffered saline (PBS) and exposed to trypan blue (0.25% in PBS). The results of the three individual experiments gave rise to the means and standard errors in each graph. The results were expressed as a percentage of untreated control.

### Direct Contact Test

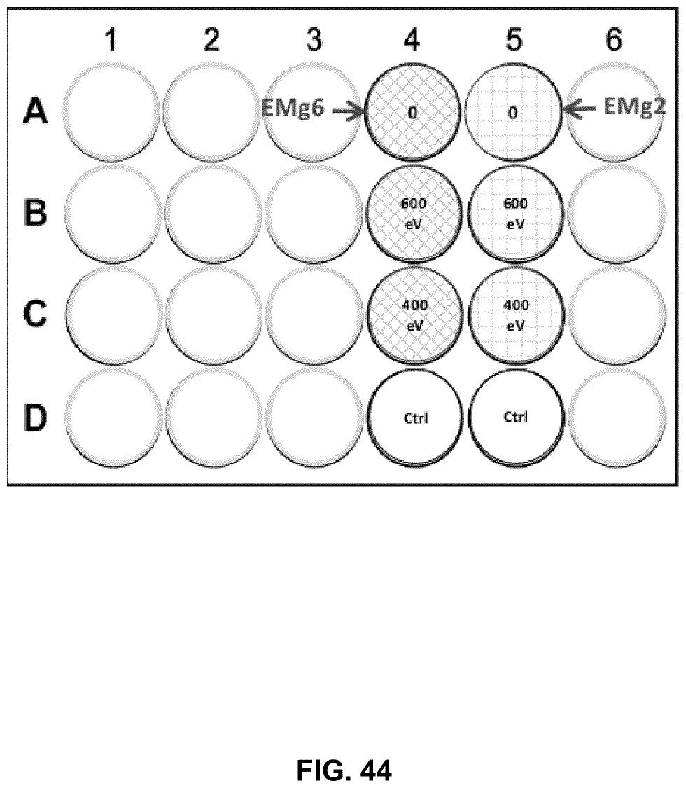

Samples were also sterilized by steam in an autoclave (3 cycles of 15 minutes at 121° C.). Then, they were soaked with culture medium prior to the testing and the cells were subcultured twice before use. Irradiated and non-irradiated samples were disposed in 24-well flat-bottomed cell culture plates. The distribution of the samples in the plate is shown in FIG. 44. Cells were seeded at 4×10⁴ cells/cm² in each well and incubated for two days to allow cell attachment on the surface of the samples. Cells were placed in standard cell culture conditions (i. e., a humidified, 5% CO², 95% air environment and 37° C.). After incubation in a humid atmosphere for 48 h, cells were fixed in PBS containing 3% formaldehyde and 0.2% glutaraldehyde. Substrates were rinsed with PBS to remove any non-adherent cells. The remaining cells morphology and adhesion location on the foams were examined by low voltage SEM at an acceleration voltage of 10 kV. Three random fields per substrate were counted.

### Results

### Corrosion Test

The corrosion experiments were designed to investigate the effects of two variable factors on two measured responses. The variable factors were ion energy (E) and exposition time (T). The E levels were selected as described above and T levels, which represent the times that the sample was exposed to a corrosive environment, were selected in a periodic order. Therefore, a 3×2 full factorial design of two factors with three levels was adopted here to determine which factors have significant effects on a response as well as how the effect of the E varies with the level of corrosive medium exposition time T. The Analysis of Variance (ANOVA) technique was used to find the significant main and interaction factors.

The effects of these factors and their interactions were measured by performing 18 experiments, the responses values for each condition are given in Table 19. Afterward, Table 21 shows the ANOVA results for Weight Loss and Table 22 is the ANOVA obtained for pH. The P-values for the effects of individual factors and their interaction are listed. For the significant level of 5%, the P values of <0.05 indicate a statistically significant and P<0.001 a statistically highly significant one. Hence, Table 21 shows that E has a highly significant effect over the Weight Loss and there is only a 0.00% chance that this could occur due to noise. T and the interaction of T and E also have significant effect on Weight Loss. In Table 22, E and T can be seen as significant factors for pH although there is not interaction between T and E. In both cases, Pores Size shows P values of >0.05 which means that the pores size does not have significant influence on Weight Loss and pH.

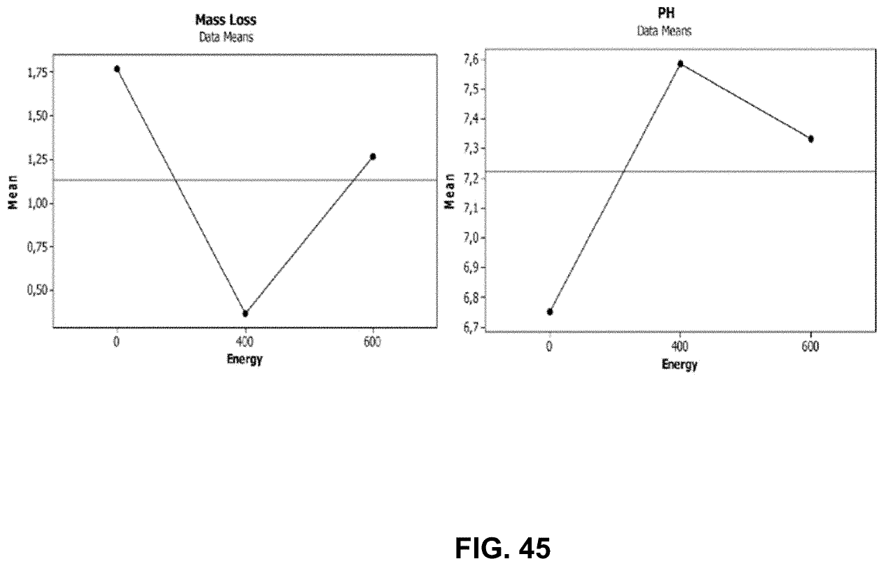

A main effects plot graphs the mean response for each factor level connected by a line. Therefore, main effects plots were used for examining the differences between level means for E. There is a main effect when different levels of a factor affect the response differently. E main effects on Weight Loss and pH were plotted in FIG. 45. It is evident that the 400 eV ion energy drastically reduces the Weight Loss while keeping the pH in a high level. 600 eV ion energy also has an effect on the reduction of Weight Loss but it is not as significant as the effect of 400 eV.

TABLE 21
ANOVA Test results for weight loss

| Source | DOF | SC src. | F | P |
|---|---|---|---|---|
| Pores Size | 1 | 0.32 | 4.34 | 0.071 |
| T | 2 | 1.90 | 12.90 | 0.003 |
| E | 2 | 6.04 | 40.95 | 0.000 |
| T*E | 4 | 1.57 | 5.31 | 0.022 |
| Error | 8 | 0.59 | | |
| Total | 17 | 10.42 | | |
| Std. dev. | 0.27 | | | |
| R-squared | 94.34% | | | |
| Adj. R-squared | 87.97% | | | |

DOF. Degrees of freedom, SC src. Sum of squares.

TABLE 22
ANOVA test results for pH

| Source | DOF | SC src. | F | P |
|---|---|---|---|---|
| Pores size | 1 | 0.056 | 0.31 | 0.594 |
| T | 2 | 11.11 | 30.77 | 0.000 |
| E | 2 | 2.19 | 6.08 | 0.025 |
| T*E | 4 | 0.81 | 1.12 | 0.413 |
| Error | 8 | 1.44 | | |
| Total | 17 | 15.61 | | |
| Std. dev. | 0.424 | | | |
| R-squared | 90.75% | | | |
| Adj. R-squared | 80.34% | | | |

DOF. Degrees of freedom, SC src. Sum of squares.

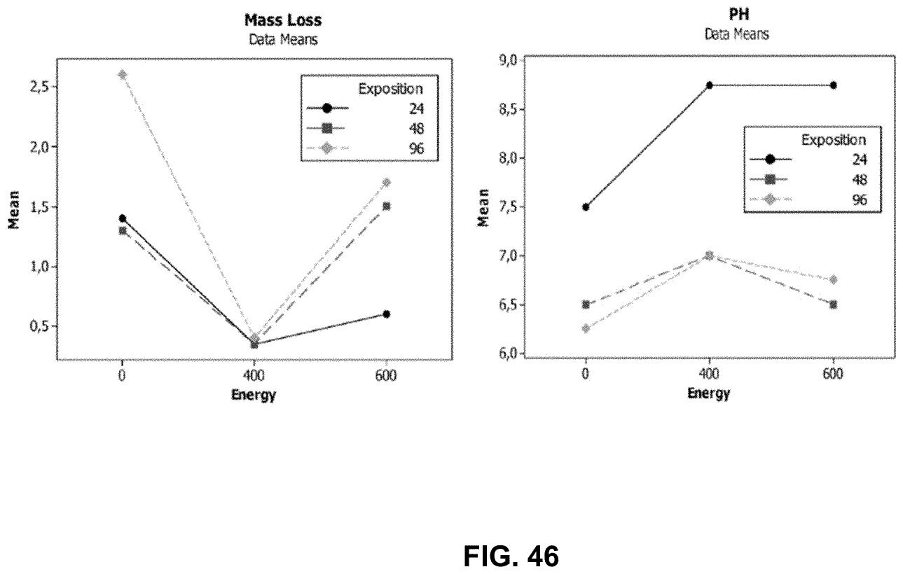

The interaction plots (FIG. 46) show the relation between E and T. As it was expected, irradiation has been used to enhance the corrosive performance of the samples in the time. From this figure, the following points can be inferred: The pH value has an inversely proportional relationship with Weight Loss, if the pH value stays in a high level, Weight Loss decreases. However, Weight Loss decreases slightly with the increase in exposure time (T). It possibly resulted from the increase in hydrogen evolution because of acidification of the medium with an increase in exposure time. This behavior could be attributed to the corrosion occurring over an increasing fraction of the surface and, consequently, the emergence of insoluble corrosion products on the surface of the alloy that could slow down the corrosion rate.

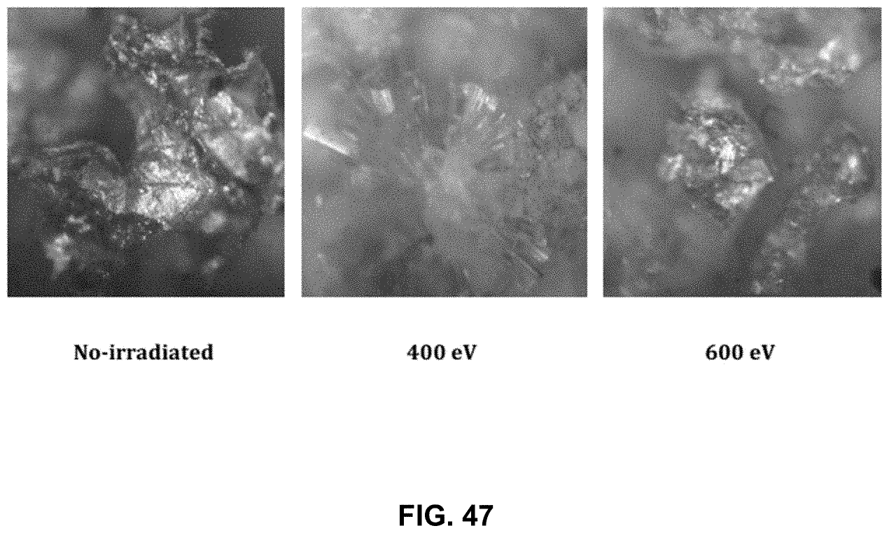

Particularly, for 400 eV ion energy, in FIG. 46, it can be seen that Weight Loss decrease with the increase in exposure time. For 600 eV ion energy the behavior was similar in the first 24 hours. Nevertheless, the increase in exposure time enhanced the tendency of the samples irradiated with 400 eV to form bigger corrosion products, which accumulated over the surface of the samples. FIG. 47, is an optical image of the corrosion products after 96 h of exposition. The microscope images reveal the presence of individual small assemblies in the samples irradiated with 400 eV. These corrosion products could depress the Weight Loss due to the passivation in the medium immersion.

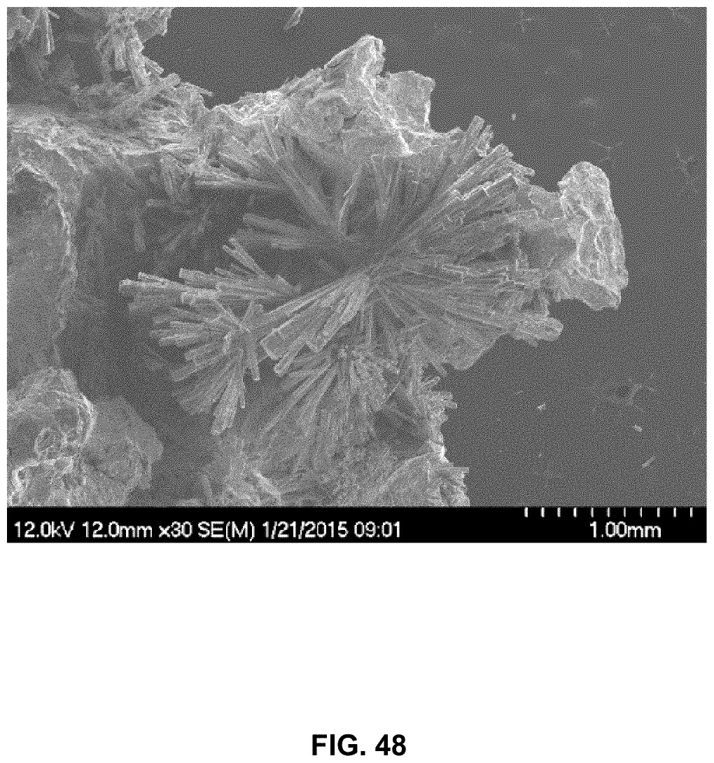

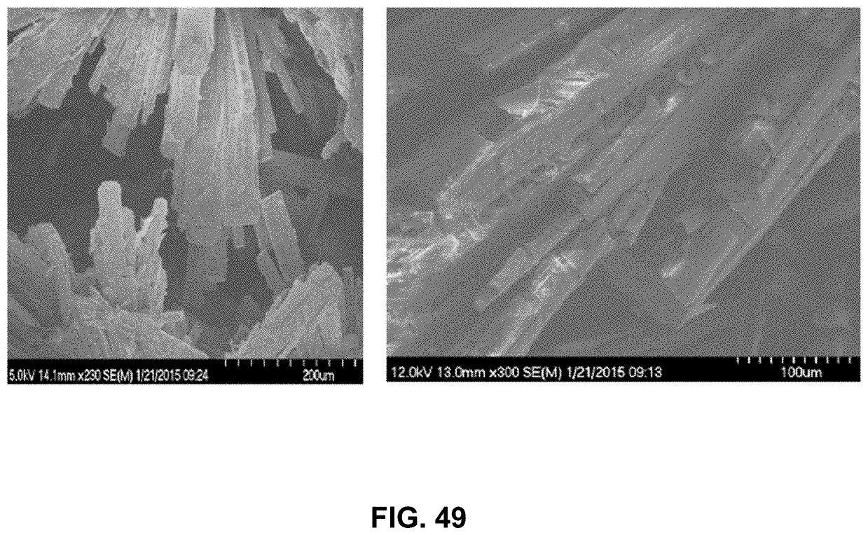

The presence of corrosion products was also assessed with SEM. The assemblies were typically around 150 μm to 900 μm in size. The flower-like features grew in size and started to merge with immediate neighbors. An enlarged view of the flower-like assemblies is presented in FIG. 48. FIG. 49 presents a detailed end view of the individual pieces showing their panel-like structure. Usually, at the end of a 96 h immersion period substrates were completely covered with this large flower-like features.

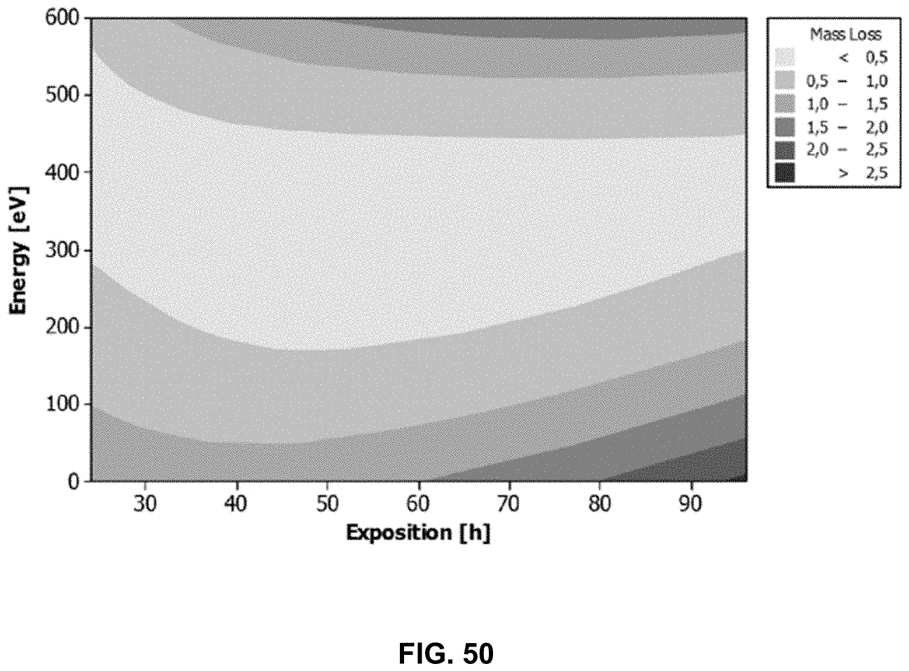

A contour plot was graphed to explore the potential relationship between Ion Energy and Weight Loss during the Exposition Time. It is a useful tool to find a region with the desired properties within the studied level range of the variable factors. In FIG. 50, it can be clearly observed that different patterns of responses are generated by the factors in the studied region. An interesting energy region has been found where the values are around 275 eV to 575 eV. These values show less Weight Loss for all the immersion times. Therefore, more experiments need to be performed with points selected from this energy region in order to obtain a more accurately control of the irradiation effect on the Mg-based foam surfaces.

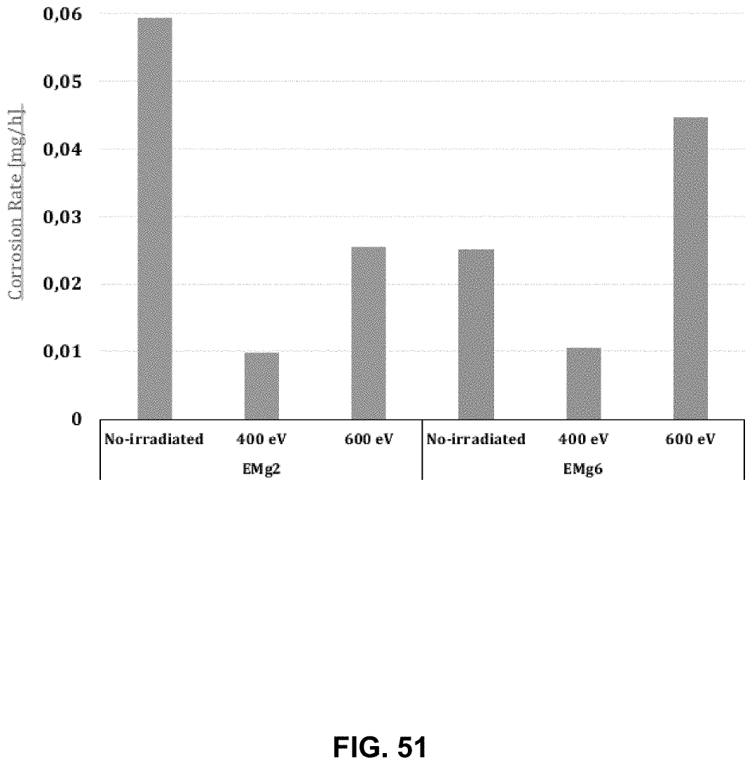

The degradation rate was calculated as the slope of the line resulting from the linear regression between Weight Loss and Exposition Time, and it was expressed as [mg/h]. The results are listed in FIG. 51.

### Viability Test

### Liquid Extract of the Material

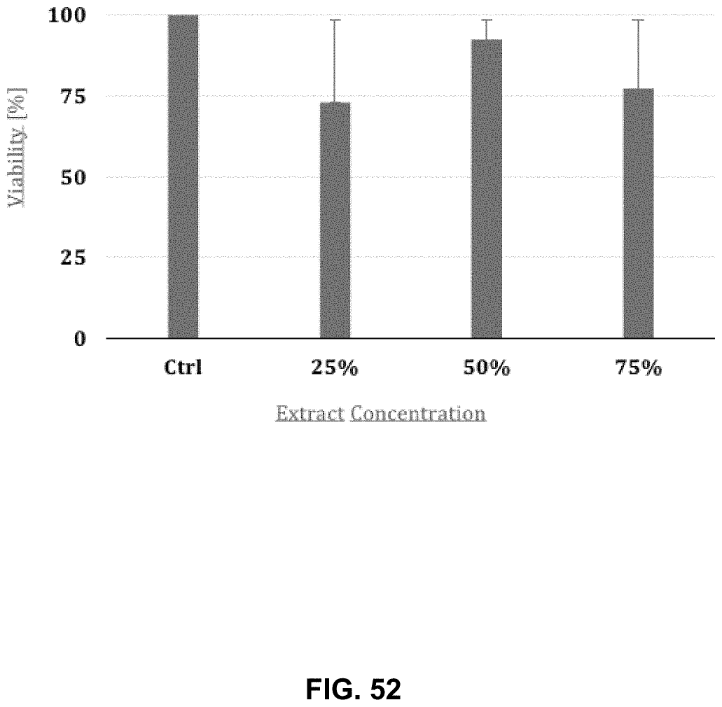

Cytotoxic action of the corrosion product of the alloy has been evaluated and expressed as the proportion of viable cells compared with untreated control cultures. Incubation with the extract of Mg-based material affected viability even at the lowest dose used. Similar proportionality between cytotoxic action of the extracts and their concentrations is evident. FIG. 52 shows that Mg-based extracts have less than 27% cytotoxicity against the cells. According to ISO 10993 5:2009 [167], there are cytotoxic effects when the reduction in cells viability is more than 30%.

### Direct Contact Test

Biocompatibility of Mg-based alloy as porous material was assessed by using an adherent cell culture to determine the cell responses on the surfaces. The remaining cells morphology and adhesion location on the foams were examined by low voltage SEM. Three random regions per substrate, with dimensions of 120 μm×90 μm, were counted. The results are showed in Table 23.

TABLE 23
Number of cells counted by surface type.

| Sample | Cells |
|---|---|
| No-irradiated | 36 |
| 400 eV | 110 |
| 600 eV | 16 |

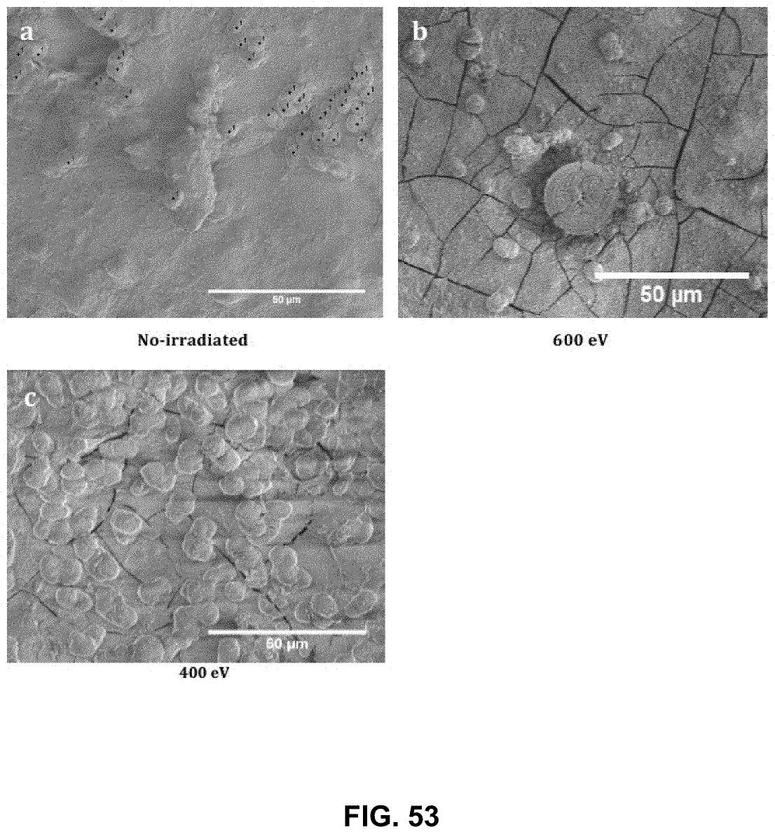

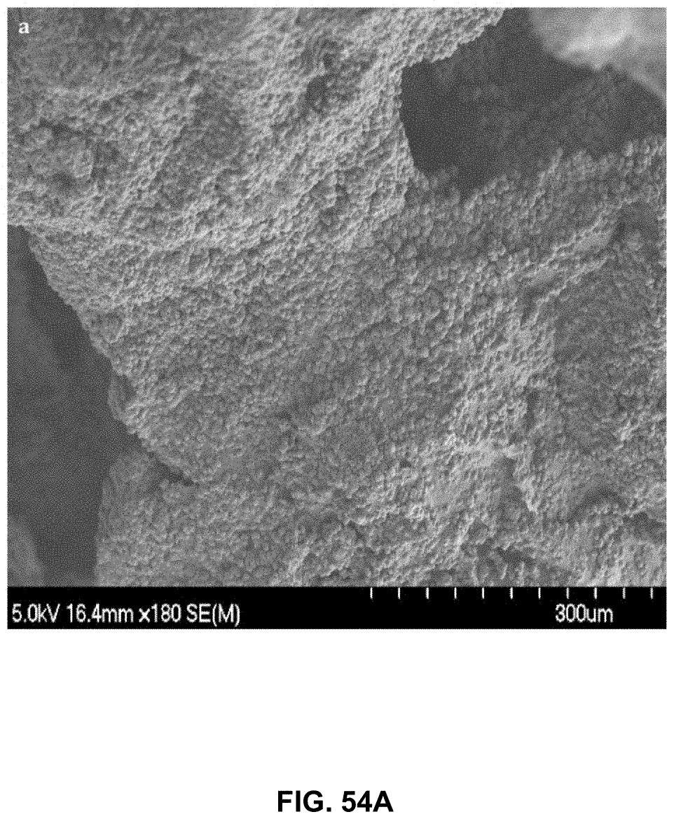

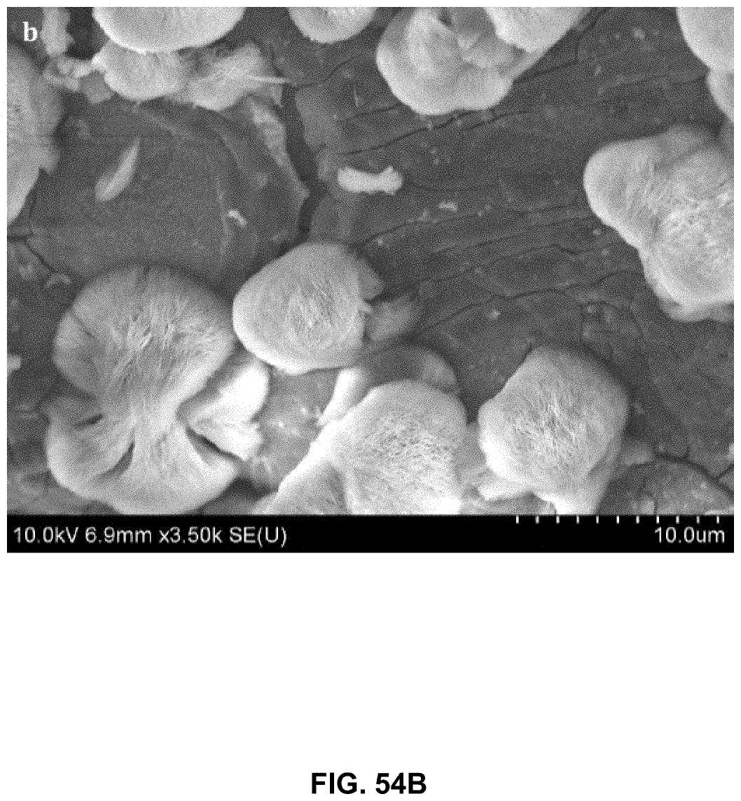

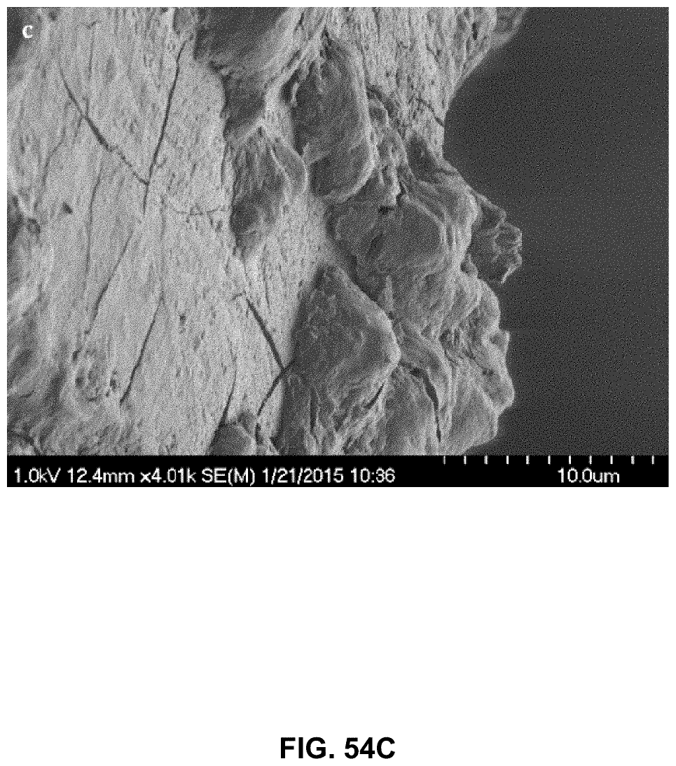

These results indicate that the number of cells attached to a 400 eV irradiated surface is around seven times bigger than on a 600 eV surface and around three times bigger than on a non-irradiated surface. FIG. 53 shows representative images of the 120 μm×90 μm region for each surface type. FIG. 54A, which corresponds to a 400 eV surface, shows that numerous cells were well attached but non-uniformly distributed on the surface. Cells were attached closely together (FIG. 54B), showing good interaction between them and corrosion products (FIG. 54C) formed on the surface.

### Discussion

Ion irradiation is a process by which ions of a material are accelerated in an electrical field and impacted onto another solid [244]. Low energy irradiation often results in pronounced modification of the near surface of a solid, a wide range of physical and chemical phenomena is associated with the interaction of low-energy ions with surfaces [245]. Therefore, electron or ion beams may be used to tailor the structure and properties of a substrate. The irradiation of a solid by particles leads to introduce disorder, and low energy ion beam irradiation can lead to the formation of structures on the surfaces of metals.

The type of effect created on the surface depends on incident ion energy, angle, current, and fluence [246]. Very low-energy range appears to have important implications, surface displacements can occur without concurrent production of bulk defects. At the very surface, ion irradiation may alter the composition. It defines defects at the surface, backscattering of incident particles, emission of electrons and photons, and ejection of target atoms and molecules (sputtering) may take place. These processes may cause extensive displacement cascades and point defects [247].

Mg-based scaffolds for bone tissue engineering were fabricated [22], [24], [25], [79], [119], [248]. In the early stage of the investigation on porous devices based on Mg alloys, the main objective is to develop manufacturing processes to create sponge-like architectures, controlled microstructures, and understanding the resulting mechanical behavior. At present state of this review few surface modifications of porous Mg have been found. Nguyen [44], applied a biomimetic calcium phosphate (CaP) coating system on porous samples to improve the corrosion resistance. Thirumalaikumarasamy et al. [238] reported a three factors, five level, design matrix that was used to evaluate the corrosion behavior of solid AZ31 Mg-based alloy. Both assays were performed in NaCl solution and corrosion resistance was assessed by mass loss measurement.

Also, Yazdimamaghani et al. [248] applied biocompatible polymeric layer reinforced with a bioactive ceramic made of polycaprolactone (PCL) and bioactive glass (BG) on the surface of Mg scaffolds. Their results showed noticeable improvement in corrosion resistance compared with the uncoated samples. Nevertheless, the surface modification proposed may provide enhanced anticorrosion property and cellular response without affecting the light weight and bulk attributes of the device.

The in vitro corrosion test protocols adopted in aforementioned studies were relatively basic, making it difficult to interpret the results in relation to degradation profiles for biomedical applications. The influence of culture medium ions on the Mg corrosion has been assessed. Yamamoto and Hiromoto [249] performed immersion tests of pure Mg into NaCl, Earle (+), E-MEM+FBS to examine the effects of inorganic salts and organic compounds on the degradation of Mg. They found a surface layer formed during the immersion dependent on the kind of the fluid, this layer controls the degradation of Mg. Thus, the use of a solution such as E-MEM+FBS, having the similar composition to blood plasma, is mandatory for in vitro estimation of the Mg degradation rate inside the human body. Also, Yunchang et al. [8] established that phosphate ions induce rapid surface passivation due to precipitation of Mg carbonate, which totally suppresses pitting corrosion. These results are in agreement with the results of other authors [250], [251].

Regarding the suitability of applying in vitro corrosion testing results in biomedical applications, Culture Medium 231 and SMGS, which has an ionic composition and concentration similar to that of human environment [252], at a constant temperature (37° C.) and pH9, was maintained in the current study to mimic the in vivo environment. Some studies have found that corrosion increases with the increase in temperature [253], and the effect of the pH in the corrosion rate has been well established [238], [254].

Some studies [255]-[257] indicate that the corrosion rate of the Mg alloy can be modified to some extend by controlling texture. The results herein agreed with those but our results do not yet confirm an increase in the roughness of the surface, nevertheless the surface modification influenced the corrosion tendency of Mg. Ion irradiations were capable of providing the most significant reduction in the corrosion rate of around 0.0098 mg/h compared to 0.0592 mg/h for the untreated samples. These results cannot be yet adequately explained within the framework of the theory. More characterization is required. Therefore, the contribution of the interaction to the process of corrosion and cell attachment is obvious.

Table 12 summarizes the cell viability of cells cultured in several Mg-based alloys. Since that, cells cultured in 50% concentrated AZ31 are more viable than most of the alloys listed, except for the Mg—Zn systems. In general, the sensitivity of cells to inorganic stimuli is different for different cell species, and the maximal stimulation occurs at different ionic concentrations. For human vascular smooth cell, Mg2+ is well tolerated and does not significantly reduce cell survival after a 24 h exposure at concentrations of up to 10 mmol/L [258]. Therefore, it is assumed that the decline in cell viability might be caused by the change of pH value rather than the released ions in the corrosion procedure.

Cells adhesion, spreading and migration are the first sequential reactions when coming into contact with a material surface, which is crucial for cells to survive [259]. The observation on cell morphology (FIG. 54) and the number of cells present on the surface (FIG. 53) reflected that the treated Mg-based foams had significant positive effect on the adhesion cells. Thus, there was a higher amount of cells adhered on the surface with better morphology compared to the naked Mg-based foam. Cell adhesion can be affected by various factors such as hydrogen gas evolution, increment of localized pH and released ions and corrosion products. Some investigations showed that Mg-based metals had stimulatory effects on the cells growing in vivo [260], [261]. Thus, good cell interaction on the surface of Mg-based foams means that the increment in pH and Mg ions produced from the foams dissolution is not expected to lead to toxic reactions during biodegradation.

### Conclusions

The effects of ion irradiation at different ion energy values on the degradation behavior of a metallic foam based on AZ31 Mg alloys have been systematically studied in culture Medium 231 and SMGS with different exposition times. Also, the cells behavior on the surface of the samples and cultured with the liquid extract of the material has been assessed.

Surface modification may provide a means to selectively enhance anticorrosion property and cellular response without affecting the required bulk attributes of Mg and its alloys. In this respect, a biocompatible and corrosion-resistant surface was the focus for this example of surface treatment. The aim was to create a hydrophobic surface by roughening the substrate with ion irradiation. The irradiated samples showed in both cases (corrosion and biological behavior) better performances than the naked samples. Finally, an energy region was proposed based on the results in FIG. 50 with values around 275 eV to 575 eV in order to gain control over the responses generated on the surface.

### Summary of Principal Results

Studies focused on the development of a metal foam for bone tissue applications are carried out and described herein. The gold target of this work was to enhance the cells interaction and corrosion behavior on the surface of pores.

The first step was described herein, and it is based on the need for a selection of an Mg-based alloy to develop a porous structure with enough corrosion resistance and biocompatibility. In this review, mainly the effect of the alloy elements on the corrosion resistance and biocompatibility was analyzed. The principal requirements for an Mg alloy were established and used for ranking the alloys. The alloy selected was AZ31 which is a widely studied material and was found to have the second highest performance index as compared with other alloys used for the same purpose. The AZ31 cytotoxicity and corrosion behavior are appropriate for bone implants and the mechanical properties are close to those of human bone. Moreover, the combination of Mg and Zn has the potential to be bioactive.

Once the Mg-based alloy was selected, the structure of the porous system was decided taking into account the bone organization. With this data, the fabrication route of the metal foam was described herein. The selection of the process variables and the structure obtained were also described. Characterization experiments were performed and it was determined that the foam with a pore size of 500 μm (EMg6) has a structure closer to bone structure. The topography observed and calculated was considered an advantage from a viewpoint of bone-implant interface, because the roughness values could direct the cells to a more osteoblast-like behavior.

Surface modification may provide a means to selectively enhance anticorrosion property and cellular response without affecting the required bulk attributes of Mg and its alloys. In this respect, a biocompatible and corrosion resistant surface was described herein. The aim was to create a hydrophobic surface by roughening the substrate with ion irradiation. Two ion energy values were selected taking into account the literature results. Irradiation affected the surface and the results showed better performance by the irradiated samples compared to naked ones. Ion irradiation has been found to have a great effect by improving corrosion resistance and cells adhesion to the surface. The best performances were obtained with energy values of 400 eV.

### Limitations

Characterization is an important issue for understanding the results and the behavior of the obtained products. Described herein is an attempt to avoid some of the limitations of the characterization was made.

It was necessary to calculate the roughness of the surface as well as the chemical composition. AFM and XPS techniques were considered, because of their reported results, as the more suitable method. However, AFM is a measuring method that can be applied to scan large areas for micro-scale roughness of surfaces with a small curvature [185]. For our highly porous materials with small contact area inside the pores, AFM can no longer measure the surface, and this limitation is common to other contact techniques for measuring the roughness.

Also, XPS was tried to be applied for measuring the chemical composition of the samples, but there is no control on the contact angle due to the configuration of the samples and after applying sputtering only Cu peaks appeared in the XPS pattern.

### REFERENCES

[1] M. Pietri and S. Lucarini, “The orthopaedic treatment of fragility fractures,” Clin. Cases Miner. Bone Metab., vol. 4, no. 2, pp. 108-116, 2007.
[2] M. Scaglione, L. Fabbri, D. Dell'Omo, F. Gambini, and G. Guido, “Long bone nonunions treated with autologous concentrated bone marrow-derived cells combined with dried bone allograft,” Musculoskelet. Surg., vol. 98, no. 2, pp. 101-106, August 2014.
[3] A. Olivares and A. Luis, “Evaluación computacional en andamios por prototipado rapido para la ingenieria de tejidos,” TDX Tesis Dr. En Xarxa, February 2012.
[4] S. Wu, X. Liu, K. W. K. Yeung, C. Liu, and X. Yang, “Biomimetic porous scaffolds for bone tissue engineering,” Mater. Sci. Eng. R Rep., vol. 80, pp. 1-36, June 2014.
[5] M. Nair and E. Elizabeth, “Applications of titania nanotubes in bone biology,” J. Nanosci. Nanotechnol., vol. 15, no. 2, pp. 939-955, 2015.
[6] S. L. Pownder, M. F. Koff, P. H. Shah, L. A. Fortier, and H. G. Potter, “Magnetic resonance imaging of an equine fracture model containing stainless steel metal implants,” 2015.
[7] Q. Chen and G. A. Thouas, “Metallic implant biomaterials,” Mater. Sci. Eng. R Rep., vol. 87, pp. 1-57, 2015.
[8] Y. Xin, K. Huo, H. Tao, G. Tang, and P. K. Chu, “Influence of aggressive ions on the degradation behavior of biomedical magnesium alloy in physiological environment,” Acta Biomater, vol. 4, no. 6, pp. 2008-2015, November 2008.
[9] A. R. Costa-Pinto, R. L. Reis, and N. M. Neves, “Scaffolds based bone tissue engineering: the role of chitosan,” Tissue Eng. Part B Rev., vol. 17, no. 5, pp. 331-347, October 2011.
[10] A. Das, D. A. Barker, T. Wang, C. M. Lau, Y. Lin, and E. A. Botchwey, “Delivery of bioactive lipids from composite microgel-microsphere injectable scaffolds enhances stem cell recruitment and skeletal repair,” PLoS ONE, vol. 9, no. 7, 2014.
[11] K. R. Remya, J. Joseph, S. Mani, A. John, H. K. Varma, and P. Ramesh, “Nanohydroxyapatite incorporated electrospun polycaprolactone/polycaprolactone-polyethyleneglycol-polycaprolactone blend scaffold for bone tissue engineering applications,” J. Biomed. Nanotechnol., vol. 9, no. 9, pp. 1483-1494, September 2013.
[12] Y. Liu, J. Lim, and S.-H. Teoh, “Review: development of clinically relevant scaffolds for vascularised bone tissue engineering,” Biotechnol. Adv., vol. 31, no. 5, pp. 688-705, October 2013.
[13] M. Montazeri, S. Karbasi, M. R. Foroughi, A. Monshi, and R. Ebrahimi-Kahrizsangi, “Evaluation of mechanical property and bioactivity of nano-bioglass 45S5 scaffold coated with poly-3-hydroxybutyrate,” J. Mater. Sci. Mater. Med., vol. 26, no. 2, p. 5369, February 2015.
[14] V. Guneta, J. K. Wang, S. Maleksaeedi, Z. He, M. T. C. Wong, and C. Choong, “Three dimensional printing of titanium for bone tissue engineering applications: A preliminary study,” J. Biomim. Biomater. Biomed. Eng., vol. 21, no. 1, pp. 101-115, 2014.
[15] Q.-H. Qin, Mechanics of cellular bone remodeling: coupled thermal, electrical, and mechanical field effects. Boca Raton, Fla.: CRC Press, 2013.
[16] T. L. Nguyen, M. P. Staiger, G. J. Dias, and T. B. F. Woodfield, “A Novel Manufacturing Route for Fabrication of Topologically-Ordered Porous Magnesium Scaffolds,” Adv. Eng. Mater., vol. 13, no. 9, pp. 872-881, 2011.
[17] G. Song and S. Song, “A Possible Biodegradable Magnesium Implant Material,” Adv. Eng. Mater., vol. 9, no. 4, pp. 298-302, April 2007.
[18] P. Neacsu, R. Ion, V. Mitran, A. Staras, and A. Cimpean, “State of the Art and Recent Patents on Mg-Based Biodegradable Bone Implants,” Recent Pat. Regen. Med., vol. 4, no. 3, pp. 168-188, February 2015.
[19] F. Witte, N. Hort, C. Vogt, S. Cohen, K. U. Kainer, R. Willumeit, and F. Feyerabend, “Degradable biomaterials based on magnesium corrosion,” Curr. Opin. Solid State Mater. Sci., vol. 12, no. 5-6, pp. 63-72, October 2008.
[20] J. Y. Rho, R. B. Ashman, and C. H. Turner, “Young's modulus of trabecular and cortical bone material: Ultrasonic and microtensile measurements,” J. Biomech., vol. 26, no. 2, pp. 111-119, February 1993.
[21] A. K. Khanra, H. C. Jung, S. H. Yu, K. S. Hong, and K. S. Shin, “Microstructure and mechanical properties of Mg-HAP composites,” Bull. Mater. Sci., vol. 33, no. 1, pp. 43-47, 2010.
[22] J. Trinidad, I. Marco, G. Arruebarrena, J. Wendt, D. Letzig, E. Saenz de Argandona, and R. Goodall, “Processing of Magnesium Porous Structures by Infiltration Casting for Biomedical Applications: Processing of Magnesium Porous Structures by . . . ,” Adv. Eng. Mater., vol. 16, no. 2, pp. 241-247, February 2014.
[23] X. Wang, Z. Li, Y. Huang, K. Wang, X. Wang, and F. Han, “Processing of magnesium foams by weakly corrosive and highly flexible space holder materials,” Mater. Des., vol. 64, pp. 324-329, December 2014.
[24] J. O. Osorio-Hernandez, M. A. Suarez, R. Goodall, G. A. Lara-Rodriguez, I. Alfonso, and I. A. Figueroa, “Manufacturing of open-cell Mg foams by replication process and mechanical properties,” Mater. Des., vol. 64, pp. 136-141, December 2014.
[25] G. Hao, F. Han, and W. Li, “Processing and mechanical properties of magnesium foams,” J. Porous Mater., vol. 16, no. 3, pp. 251-256, June 2009.
[26] Z. S. Seyedraoufi and S. Mirdamadi, “Synthesis, microstructure and mechanical properties of porous Mg□Zn scaffolds,” J. Mech. Behay. Biomed. Mater., vol. 21, no. 0, pp. 1-8, May 2013.
[27] H. Zhuang, Y. Han, and A. Feng, “Preparation, mechanical properties and in vitro biodegradation of porous magnesium scaffolds,” Mater. Sci. Eng. C, vol. 28, no. 8, pp. 1462-1466, December 2008.
[28] A. Oryan, S. Alidadi, A. Moshiri, and N. Maffulli, “Bone regenerative medicine: classic options, novel strategies, and future directions,” J. Orthop. Surg., vol. 9, no. 1, p. 18, March 2014.
[29] I. Manjubala, A. Woesz, C. Pilz, M. Rumpler, N. Fratzl-Zelman, P. Roschger, J. Stampfl, and P. Fratzl, “Biomimetic mineral-organic composite scaffolds with controlled internal architecture,” J. Mater. Sci. Mater. Med., vol. 16, no. 12, pp. 1111-1119, December 2005.
[30] S. K. Nandi, S. Roy, P. Mukherjee, B. Kundu, D. K. De, and D. Basu, “Orthopaedic applications of bone graft & graft substitutes: a review,” Indian J. Med. Res., vol. 132, pp. 15-30, July 2010.
[31] Y. Shibata and Y. Tanimoto, “A review of improved fixation methods for dental implants. Part I: Surface optimization for rapid osseointegration,” J. Prosthodont. Res., vol. 59, no. 1, pp. 20-33, 2015.
[32] A. R. Boccaccini, U. Kneser, and A. Arkudas, “Scaffolds for vascularized bone regeneration: advances and challenges,” Expert Rev. Med. Devices, vol. 9, no. 5, pp. 457-460, September 2012.
[33] V. Karageorgiou and D. Kaplan, “Porosity of 3D biomaterial scaffolds and osteogenesis,” Biomaterials, vol. 26, no. 27, pp. 5474-5491, September 2005.
[34] I. H. Kalfas, “Principles of bone healing,” Neurosurg. Focus, vol. 10, no. 4, p. El, 2001.
[35] E. N. Marieb, Essentials of Human Anatomy & Physiology. Pearson Education, 2014.
[36] G. J. Tortora and B. H. Derrickson, Principles of Anatomy and Physiology, 13th edition. Hoboken, N.J.: Wiley, 2011.
[37] H. Altenbach, “Qin, Qing-Hua: Mechanics of Cellular Bone Remodeling—Coupled Thermal, Electrical, and Mechanical Field Effects. CRC Press: Taylor & Francis Group, Boca Raton, London, New York,” ZAMM—J. Appl. Math. Mech. Z. Für Angew. Math. Mech., vol. 93, no. 5, pp. 345-345, May 2013.
[38] C. Aparicio Bádenas, “Tratamientos de superficie sobre titanio comercialmente puro para la mejora de la osteointegración de los implantes dentales,” TDX (Tesis Doctorals en Xarxa), 15 Apr. 2005. [Online]. Available: http://www.tdx.cat/handle/10803/6044. [Accessed: 9 Apr. 2015].
[39] S. J. Hazelwood, R. Bruce Martin, M. M. Rashid, and J. J. Rodrigo, “A mechanistic model for internal bone remodeling exhibits different dynamic responses in disuse and overload,” J. Biomech., vol. 34, no. 3, pp. 299-308, March 2001.
[40] R. B. Martin, “The usefulness of mathematical models for bone remodeling,” Am. J. Phys. Anthropol., vol. 28, no. S6, pp. 227-236, January 1985.
[41] R. Gunzburg, M. Szpalski, N. Passuti, and M. Aebi, The Use of Bone Substitutes in Spine Surgery: A State of the Art Review. Springer Science & Business Media, 2001.
[42] S. M. Kurtz and J. N. Devine, “PEEK Biomaterials in Trauma, Orthopedic, and Spinal Implants,” Biomaterials, vol. 28, no. 32, pp. 4845-4869, November 2007.
[43] W. M. Haynes, Ed., CRC Handbook of Chemistry and Physics, 95th Edition, 95 edition. Boca Raton, Fla.: CRC Press, 2014.
[44] T. Nguyen, “Synthesis of topologically-ordered porous magnesium,” 2011.
[45] N.-E. L. Saris, E. Mervaala, H. Karppanen, J. A. Khawaja, and A. Lewenstam, “Magnesium: An update on physiological, clinical and analytical aspects,” Clin. Chim. Acta, vol. 294, no. 1-2, pp. 1-26, April 2000.
[46] M. M. Avedesian and H. Baker, ASM Specialty Handbook: Magnesium and Magnesium Alloys. ASM International, 1999.
[47] P. D. M. Z. Iqbal, Elements in Health and Disease. Dr. Ahsan lqbal.
[48] C. Fox, D. Ramsoomair, and C. Carter, “Magnesium: its proven and potential clinical significance,” South. Med. J., vol. 94, no. 12, pp. 1195-1201, December 2001.
[49] W. Jahnen-Dechent and M. Ketteler, “Magnesium basics,” Clin. Kidney J., vol. 5, no. Suppl 1, pp. i3-i14, January 2012.
[50] A. Romani and A. Scarpa, “Regulation of cell magnesium,” Arch. Biochem. Biophys., vol. 298, no. 1, pp. 1-12, October 1992.
[51] K. W. Beyenbach, “Transport of magnesium across biological membranes,” Magnes. Trace Elem., vol. 9, no. 5, pp. 233-254, 1990.
[52] R. Swaminathan, “Magnesium Metabolism and its Disorders,” Clin. Biochem. Rev., vol. 24, no. 2, pp. 47-66, May 2003.
[53] M. J. Arnaud, “Update on the assessment of magnesium status,” Br. J. Nutr., vol. 99 Suppl 3, pp. S24-36, June 2008.
[54] S. Castiglioni, A. Cazzaniga, W. Albisetti, and J. A. M. Maier, “Magnesium and Osteoporosis: Current State of Knowledge and Future Research Directions,” Nutrients, vol. 5, no. 8, pp. 3022-3033, July 2013.
[55] N. Li and Y. Zheng, “Novel Magnesium Alloys Developed for Biomedical Application: A Review,” J. Mater. Sci. Technol., vol. 29, no. 6, pp. 489-502, June 2013.
[56] Y. Chen, Z. Xu, C. Smith, and J. Sankar, “Recent advances on the development of magnesium alloys for biodegradable implants,” Acta Biomater., vol. 10, no. 11, pp. 4561-4573, November 2014.
[57] Y. Ding, C. Wen, P. Hodgson, and Y. Li, “Effects of alloying elements on the corrosion behavior and biocompatibility of biodegradable magnesium alloys: a review,” J. Mater. Chem. B, vol. 2, no. 14, pp. 1912-1933, March 2014.
[58] T. Schlich, The Origins of Organ Transplantation: Surgery and Laboratory Science, 1880-1930. University Rochester Press, 2010.
[59] D. A.-Z. e.V and C. Kammer, Magnesium-Taschenbuch, Auflage: 1. Düsseldorf: Alu Media, 2000.
[60] M. Haude, R. Erbel, P. Erne, S. Verheye, H. Degen, D. Böse, P. Vermeersch, I. Wijnbergen, N. Weissman, F. Prati, R. Waksman, and J. Koolen, “Safety and performance of the drug-eluting absorbable metal scaffold (DREAMS) in patients with de-novo coronary lesions: 12 month results of the prospective, multicentre, first-in-man BIOSOLVE-I trial,” Lancet, vol. 381, no. 9869, pp. 836-844, March 2013.
[61] H. Hermawan, D. Dube, and D. Mantovani, “Developments in metallic biodegradable stents,” Acta Biomater., vol. 6, no. 5, pp. 1693-1697, May 2010.
[62] F. Witte, “The history of biodegradable magnesium implants: A review,” Acta Biomater., vol. 6, no. 5, pp. 1680-1692, May 2010.
[63] M. P. Staiger, A. M. Pietak, J. Huadmai, and G. Dias, “Magnesium and its alloys as orthopedic biomaterials: A review,” Biomaterials, vol. 27, no. 9, pp. 1728-1734, March 2006.
[64] M. Navarro, A. Michiardi, O. Castano, and J.. Planell, “Biomaterials in orthopaedics,” J. R. Soc. Interface, vol. 5, no. 27, pp. 1137-1158, October 2008.
[65] F. Witte, H. Ulrich, M. Rudert, and E. Willbold, “Biodegradable magnesium scaffolds: Part 1: appropriate inflammatory response,” J. Biomed. Mater. Res. A, vol. 81, no. 3, pp. 748-756, June 2007.
[66] H. U. F Witte, “Biodegradable magnesium scaffolds: Part II: peri-implant bone remodeling.,” J. Biomed. Mater. Res. A, vol. 81, no. 3, pp. 757-65, 2007.
[67] X.-N. Gu, S.-S. Li, X.-M. Li, and Y.-B. Fan, “Magnesium based degradable biomaterials: A review,” Front. Mater. Sci., vol. 8, no. 3, pp. 200-218, September 2014.
[68] N. T. Kirkland, N. Birbilis, and M. P. Staiger, “Assessing the corrosion of biodegradable magnesium implants: A critical review of current methodologies and their limitations,” Acta Biomater., vol. 8, no. 3, pp. 925-936, March 2012.
[69] A. S. Barling, “A New Ligature,” Br. Med. J., vol. 2, no. 2278, p. 473, August 1904.
[70] A. A. Zierold, Reaction of bone to various metals. Chicago: American Medical Association, 1924.
[71] V. S. Borodkin, E. M. Savitsky, K. M. Sivash, G. B. Stroganov, V. F. Terekhova, N. M. Tikhova, and M. V. Volkov, “Magnesium-base alloy for use in bone surgery,” U.S. Pat. No. 3,687,135 A, 29 Aug. 1972.
[72] J. D. Cao, N. T. Kirkland, K. J. Laws, N. Birbilis, and M. Ferry, “Ca—Mg—Zn bulk metallic glasses as bioresorbable metals,” Acta Biomater., vol. 8, no. 6, pp. 2375-2383, 2012.
[73] X.-B. Chen, N. T. Kirkland, H. Krebs, M.-A. Thiriat, S. Virtanen, D. Nisbet, and N. Birbilis, “In vitro corrosion survey of Mg-xCa and Mg-3Zn-yCa alloys with and without calcium phosphate conversion coatings,” Corros. Eng. Sci. Technol., vol. 47, no. 5, pp. 365-373, 2012.
[74] N. T. Kirkland, J. Lespagnol, N. Birbilis, and M. P. Staiger, “A survey of bio-corrosion rates of magnesium alloys,” Corros. Sci., vol. 52, no. 2, pp. 287-291, 2010.
[75] J. Walker, S. Shadanbaz, N. T. Kirkland, E. Stace, T. Woodfield, M. P. Staiger, and G. J. Dias, “Magnesium alloys: Predicting in vivo corrosion with in vitro immersion testing,” J. Biomed. Mater. Res.—Part B Appl. Biomater., vol. 100 B, no. 4, pp. 1134-1141, 2012.
[76] F. Witte, J. Fischer, J. Nellesen, H.-A. Crostack, V. Kaese, A. Pisch, F. Beckmann, and H. Windhagen, “In vitro and in vivo corrosion measurements of magnesium alloys,” Biomaterials, vol. 27, no. 7, pp. 1013-1018, 2006.
[77] F. Witte, V. Kaese, H. Haferkamp, E. Switzer, A. Meyer-Lindenberg, C. J. Wirth, and H. Windhagen, “In vivo corrosion of four magnesium alloys and the associated bone response,” Biomaterials, vol. 26, no. 17, pp. 3557-3563, June 2005.
[78] F. Witte, N. Hort, and F. Feyerabend, “Biodegradable Magnesium Implants-How Do They Corrode in-vivo?,” in Magnesium Technology 2011, 2011, pp. 17-17.
[79] K. Bobe, E. Willbold, I. Morgenthal, O. Andersen, T. Studnitzky, J. Nellesen, W. Tillmann, C. Vogt, K. Vano, and F. Witte, “In vitro and in vivo evaluation of biodegradable, open-porous scaffolds made of sintered magnesium W4 short fibres,” Acta Biomater., vol. 9, no. 10, pp. 8611-8623, 2013.
[80] J. Kuhlmann, I. Bartsch, E. Willbold, S. Schuchardt, O. Holz, N. Hort, D. Hoche, W. R. Heineman, and F. Witte, “Fast escape of hydrogen from gas cavities around corroding magnesium implants,” Acta Biomater., vol. 9, no. 10, pp. 8714-8721, 2013.
[81] W.-C. Kim, J.-G. Kim, J.-Y. Lee, and H.-K. Seok, “Influence of Ca on the corrosion properties of magnesium for biomaterials,” Mater. Lett., vol. 62, no. 25, pp. 4146-4148, September 2008.
[82] Z. G. Huan, M. A. Leeflang, J. Zhou, L. E. Fratila-Apachitei, and J. Duszczyk, “In vitro degradation behavior and cytocompatibility of Mg—Zn—Zr alloys,” J. Mater. Sci. Mater. Med., vol. 21, no. 9, pp. 2623-2635, September 2010.
[83] S. Zhang, X. Zhang, C. Zhao, J. Li, Y. Song, C. Xie, H. Tao, Y. Zhang, Y. He, Y. Jiang, and Y. Bian, “Research on an Mg—Zn alloy as a degradable biomaterial,” Acta Biomater., vol. 6, no. 2, pp. 626-640, February 2010.
[84] D. Chen, Y. He, H. Tao, Y. Zhang, Y. Jiang, X. Zhang, and S. Zhang, “Biocompatibility of magnesium-zinc alloy in biodegradable orthopedic implants,” Int. J. Mol. Med., vol. 28, no. 3, pp. 343-348, 2011.
[85] P. Han, M. Tan, S. Zhang, W. Ji, J. Li, X. Zhang, C. Zhao, Y. Zheng, and Y. Chai, “Shape and site dependent in vivo degradation of Mg—Zn pins in rabbit femoral condyle,” Int. J. Mol. Sci., vol. 15, no. 2, pp. 2959-2970, 2014.
[86] Y. Chen, J. Yan, X. Wang, S. Yu, Z. Wang, X. Zhang, S. Zhang, Y. Zheng, C. Zhao, and Q. Zheng, “In vivo and in vitro evaluation of effects of Mg-6Zn alloy on apoptosis of common bile duct epithelial cell,” 2014.
[87] X. P. Zhang, Z. P. Zhao, F. M. Wu, Y. L. Wang, and J. Wu, “Corrosion and wear resistance of AZ91D magnesium alloy with and without microarc oxidation coating in Hank's solution,” J. Mater. Sci., vol. 42, no. 20, pp. 8523-8528, 2007.
[88] S. Hiromoto and A. Yamamoto, “Control of degradation rate of bioabsorbable magnesium by anodization and steam treatment,” Mater. Sci. Eng. C, vol. 30, no. 8, pp. 1085-1093, 2010.
[89] T. S. N. Sankara Narayanan, I. S. Park, and M. H. Lee, “Strategies to improve the corrosion resistance of microarc oxidation (MAO) coated magnesium alloys for degradable implants: Prospects and challenges,” Prog. Mater. Sci., vol. 60, no. 1, pp. 1-71, 2014.
[90] P. Shi, B. Niu, S. E, Y. Chen, and Q. Li, “Preparation and characterization of PLA coating and PLA/MAO composite coatings on AZ31 magnesium alloy for improvement of corrosion resistance,” Surf. Coat. Technol., vol. 262, pp. 26-32, 2015.
[91] T. Yan, L. Tan, D. Xiong, X. Liu, B. Zhang, and K. Yang, “Fluoride treatment and in vitro corrosion behavior of an AZ31B magnesium alloy,” Mater. Sci. Eng. C, vol. 30, no. 5, pp. 740-748, 2010.
[92] J. N. Li, P. Cao, X. N. Zhang, S. X. Zhang, and Y. H. He, “In vitro degradation and cell attachment of a PLGA coated biodegradable Mg-6Zn based alloy,” J. Mater. Sci., vol. 45, no. 22, pp. 6038-6045, 2010.
[93] H. M. Wong, K. W. K. Yeung, K. O. Lam, V. Tam, P. K. Chu, K. D. K. Luk, and K. M. C. Cheung, “A biodegradable polymer-based coating to control the performance of magnesium alloy orthopaedic implants,” Biomaterials, vol. 31, no. 8, pp. 2084-2096, March 2010.
[94] W. Zhou, T. Shen, and N. N. Aung, “Effect of heat treatment on corrosion behaviour of magnesium alloy AZ91D in simulated body fluid,” Corros. Sci., vol. 52, no. 3, pp. 1035-1041, March 2010.
[95] J. X. Yang, F. Z. Cui, I.-S. Lee, Y. P. Jiao, Q. S. Yin, and Y. Zhang, “Ion-beam assisted deposited C—N coating on magnesium alloys,” Surf. Coat. Technol., vol. 202, no. 22-23, pp. 5737-5741, 2008.
[96] J. J. Li, D. L. Kaplan, and H. Zreiqat, “Scaffold-based regeneration of skeletal tissues to meet clinical challenges,” J. Mater. Chem. B, vol. 2, no. 42, pp. 7272-7306, 2014.
[97] G. Ryan, A. Pandit, and D. P. Apatsidis, “Fabrication methods of porous metals for use in orthopaedic applications,” Biomaterials, vol. 27, no. 13, pp. 2651-2670, May 2006.
[98] J. I. Dawson and R. O. C. Oreffo, “Bridging the regeneration gap: stem cells, biomaterials and clinical translation in bone tissue engineering,” Arch. Biochem. Biophys., vol. 473, no. 2, pp. 124-131, May 2008.
[99] T. Albrektsson and C. Johansson, “Osteoinduction, osteoconduction and osseointegration,” Eur. Spine J. Off Publ. Eur. Spine Soc. Eur. Spinal Deform. Soc. Eur. Sect. Cerv. Spine Res. Soc., vol. 10 Suppl 2, pp. S96-101, October 2001.
H. Kienapfel, C. Sprey, A. Wilke, and P. Griss, “Implant fixation by bone ingrowth,” J. Arthroplasty, vol. 14, no. 3, pp. 355-368, April 1999.
F. Matassi, A. Botti, L. Sirleo, C. Carulli, and M. Innocenti, “Porous metal for orthopedics implants,” Clin. Cases Miner. Bone Metab., vol. 10, no. 2, pp. 111-115, 2013.
H. Nakajima, Porous Metals with Directional Pores. Springer Science & Business Media, 2013.
A. A. Zadpoor, “Bone tissue regeneration: the role of scaffold geometry,” Biomater. Sci., vol. 3, no. 2, pp. 231-245, January 2015.
S. Yang, K. F. Leong, Z. Du, and C. K. Chua, “The design of scaffolds for use in tissue engineering. Part I. Traditional factors,” Tissue Eng., vol. 7, no. 6, pp. 679-689, December 2001.
M. Chatzinikolaidou, S. Rekstyte, P. Danilevicius, C. Pontikoglou, H. Papadaki, M. Farsari, and M. Vamvakaki, “Adhesion and growth of human bone marrow mesenchymal stem cells on precise-geometry 3D organic-inorganic composite scaffolds for bone repair,” Mater. Sci. Eng. C, vol. 48, pp. 301-309, March 2015.
P. Kasten, I. Beyen, P. Niemeyer, R. Luginbühl, M. Bohner, and W. Richter, “Porosity and pore size of β-tricalcium phosphate scaffold can influence protein production and osteogenic differentiation of human mesenchymal stem cells: An in vitro and in vivo study,” Acta Biomater., vol. 4, no. 6, pp. 1904-1915, November 2008.
D. Logeart-Avramoglou, F. Anagnostou, R. Bizios, and H. Petite, “Engineering bone: challenges and obstacles,” J. Cell. Mol. Med., vol. 9, no. 1, pp. 72-84, March 2005.
K. Kim, D. Dean, J. Wallace, R. Breithaupt, A. G. Mikos, and J. P. Fisher, “The influence of stereolithographic scaffold architecture and composition on osteogenic signal expression with rat bone marrow stromal cells,” Biomaterials, vol. 32, no. 15, pp. 3750-3763, May 2011.
M. Bohner and F. Baumgart, “Theoretical model to determine the effects of geometrical factors on the resorption of calcium phosphate bone substitutes,” Biomaterials, vol. 25, no. 17, pp. 3569-3582, August 2004.
T. D. Roy, J. L. Simon, J. L. Ricci, E. D. Rekow, V. P. Thompson, and J. R. Parsons, “Performance of degradable composite bone repair products made via three-dimensional fabrication techniques,” J. Biomed. Mater. Res. A, vol. 66, no. 2, pp. 283-291, August 2003.
M. C. Kruyt, J. D. de Bruijn, C. E. Wilson, F. C. Oner, C. A. van Blitterswijk, A. J. Verbout, and W. J. A. Dhert, “Viable osteogenic cells are obligatory for tissue-engineered ectopic bone formation in goats,” Tissue Eng., vol. 9, no. 2, pp. 327-336, April 2003.
T. M. G. Chu, D. G. Orton, S. J. Hollister, S. E. Feinberg, and J. W. Halloran, “Mechanical and in vivo performance of hydroxyapatite implants with controlled architectures,” Biomaterials, vol. 23, no. 5, pp. 1283-1293, March 2002.
K. U. Lewandrowski, J. D. Gresser, S. Bondre, A. E. Silva, D. L. Wise, and D. J. Trantolo, “Developing porosity of poly(propylene glycol-co-fumaric acid) bone graft substitutes and the effect on osteointegration: a preliminary histology study in rats,” J. Biomater Sci. Polym. Ed., vol. 11, no. 8, pp. 879-889, 2000.
F. Simancik, “Introduction: The Strange World of Cellular Metals,” in Handbook of Cellular Metals, H.-P. Degischer and B. Kriszt, Eds. Wiley-VCH Verlag GmbH & Co. KGaA, 2002, pp. 1-4.
J. Banhart, “Manufacture, characterisation and application of cellular metals and metal foams,” Prog. Mater. Sci., vol. 46, no. 6, pp. 559-632, 2001.
P. Fernández, L. J. Cruz, and J. Coleto, “Procesos de fabricación de metales celulares. Parte II: Via sólida, deposición de metales, otros procesos,” Rev. Metal., vol. 45, no. 2, pp. 124-142, April 2009.
L. J. C. P. Fernandez, “Procesos de fabricación de metales celulares. Parte I: Procesos por via liquida,” Rev. Metal., 2008.
T. S. Srivatsan, Processing and Fabrication of Advanced Materials, XVII: Part 8: Polymer-based composites and nano composites: Volume Two. I. K. International Pvt Ltd, 2009.
G. Jiang and G. He, “A new approach to the fabrication of porous magnesium with well-controlled 3D pore structure for orthopedic applications,” Mater. Sci. Eng. C Mater. Biol. Appl., vol. 43, pp. 317-320, October 2014.
X. N. Gu, W. R. Zhou, Y. F. Zheng, Y. Liu, and Y. X. Li, “Degradation and cytotoxicity of lotus-type porous pure magnesium as potential tissue engineering scaffold material,” Mater. Lett., vol. 64, no. 17, pp. 1871-1874, September 2010.
N. T. Kirkland, I. Kolbeinsson, T. Woodfield, G. J. Dias, and M. P. Staiger, “Synthesis and properties of topologically ordered porous magnesium,” 2nd Symp. Biodegrad. Met., vol. 176, no. 20, pp. 1666-1672, December 2011.
F. Witte, J. Reifenrath, P. P. Müller, H.-A. Crostack, J. Nellesen, F. W. Bach, D. Bormann, and M. Rudert, “Cartilage repair on magnesium scaffolds used as a subchondral bone replacement,” Mater. Werkst., vol. 37, no. 6, pp. 504-508, 2006.
J. Shen, F.-B. Gong, T. Zhang, G.-Q. You, and P.-P. He, “Research progress of anti-corrosion of magnesium alloys surface,” Gongneng CailiaoJoumal Funct. Mater., vol. 45, no. 17, pp. 17022-17028 and 17033, 2014.
G. Wu, J. M. Ibrahim, and P. K. Chu, “Surface design of biodegradable magnesium alloys—A review,” Mech. Tribol. Prop. Biomed. Coat. Surf-Modif Biomater., vol. 233, no. 0, pp. 2-12, October 2013.
Y. Pan, S. He, D. Wang, D. Huang, T. Zheng, S. Wang, P. Dong, and C. Chen, “In vitro degradation and electrochemical corrosion evaluations of microarc oxidized pure Mg, Mg—Ca and Mg—Ca—Zn alloys for biomedical applications,” Mater. Sci. Eng. C, vol. 47, pp. 85-96, February 2015.
X. Xiao, H. Yu, Q. Zhu, G. Li, Y. Qu, and R. Gu, “In Vivo Corrosion Resistance of Ca—P Coating on AZ60 Magnesium Alloy,” J. Bionic Eng., vol. 10, no. 2, pp. 156-161, April 2013.
L.-H. Li, T. S. N. Sankara Narayanan, Y. K. Kim, Y.-M. Kong, I. S. Park, T. S. Bae, and M. H. Lee, “Deposition of microarc oxidation-polycaprolactone duplex coating to improve the corrosion resistance of magnesium for biodegradable implants,” Thin Solid Films, vol. 562, pp. 561-567, 2014.
G. Wu, L. Gong, K. Feng, S. Wu, Y. Zhao, and P. K. Chu, “Rapid degradation of biomedical magnesium induced by zinc ion implantation,” Mater. Lett., vol. 65, no. 4, pp. 661-663, 2011.
E. S. N. Lopes, R. J. Contieri, S. T. Button, and R. Caram, “Femoral hip stem prosthesis made of graded elastic modulus metastable β Ti Alloy,” Mater. Des., vol. 69, pp. 30-36, 2015.
L. Li, J. Gao, and Y. Wang, “Evaluation of cyto-toxicity and corrosion behavior of alkali-heat-treated magnesium in simulated body fluid,” Surf. Coat. Technol., vol. 185, no. 1, pp. 92-98, July 2004.
X. Gu, Y. Zheng, Y. Cheng, S. Zhong, and T. Xi, “In vitro corrosion and biocompatibility of binary magnesium alloys,” Biomaterials, vol. 30, no. 4, pp. 484-498, February 2009.
D. Persaud-Sharma and A. McGoron, “Biodegradable Magnesium Alloys: A Review of Material Development and Applications,” J. Biomim. Biomater. Tissue Eng., vol. 12, pp. 25-39, February 2012.
B07 Committee, “Practice for Temper Designations of Magnesium Alloys, Cast and Wrought,” ASTM International, 2014.
E07 Committee, “Guide for Computed Tomography (CT) Imaging,” ASTM International, 2011.
M. Bamberger and G. Dehm, “Trends in the Development of New Mg Alloys,” Annu. Rev. Mater. Res., vol. 38, no. 1, pp. 505-533, 2008.
T. Ruedi and R. Buckley, AO Principles of Fracture Management, Books and DVD. Thieme, 2007.
M. Erinc, W. H. Sillekens, R. G. T. M. Mannens, R. J. Werkhoven, and TNO Science and Industry, De Rondom 1, P.O. Box 6235, 5600 HE Eindhoven, Netherlands, “Applicability of existing magnesium alloys as biomedical implant materials.” 1 Jan. 2009.
X. Xia, X. Chen, W. Zhao, H. Xue, B. Liao, B. Hur, and Z. Wang, “Corrosion behavior of closed-cell AZ31 Mg alloy foam in NaCl aqueous solutions,” Corros. Sci., vol. 80, pp. 247-256, March 2014.
Z. Wen, C. Wu, C. Dai, and F. Yang, “Corrosion behaviors of Mg and its alloys with different Al contents in a modified simulated body fluid,” J. Alloys Compd., vol. 488, no. 1, pp. 392-399, November 2009.
Magnesium Alloys Containing Rare Earth Metals: Structure and Properties.
V. Kumar, Govind, K. Philippe, R. Shekhar, and K. Balani, “Processing and Nano-mechanical Characterization of Mg—Li—Al based Alloys,” Procedia Mater. Sci., vol. 5, pp. 585-591, 2014.
Y. Song, E.-H. Han, D. Shan, C. D. Yim, and B. S. You, “The role of second phases in the corrosion behavior of Mg-5Zn alloy,” Corros. Sci., vol. 60, pp. 238-245, July 2012.
F. Rosalbino, S. De Negri, A. Saccone, E. Angelini, and S. Delfino, “Bio-corrosion characterization of Mg—Zn—X (X=Ca, Mn, Si) alloys for biomedical applications,” J. Mater. Sci. Mater. Med., vol. 21, no. 4, pp. 1091-1098, April 2010.
G. Pande, Ragamouni, Li, Kumar, N. Harishankar, P. Hodgson, C. Wen, and Mushahary, “Zirconium, calcium, and strontium contents in magnesium based biodegradable alloys modulate the efficiency of implant-induced osseointegration,” Int. J. Nanomedicine, p. 2887, August 2013.
E. Zhang, L. Yang, J. Xu, and H. Chen, “Microstructure, mechanical properties and bio-corrosion properties of Mg—Si(—Ca, Zn) alloy for biomedical application,” Acta Biomater., vol. 6, no. 5, pp. 1756-1762, May 2010.
N. Hort, Y. Huang, D. Fechner, M. Störmer, C. Blawert, F. Witte, C. Vogt, H. Drucker, R. Willumeit, K. U. Kainer, and F. Feyerabend, “Magnesium alloys as implant materials—Principles of property design for Mg—RE alloys,” THERMEC'2009 Biodegrad. Met., vol. 6, no. 5, pp. 1714-1725, May 2010.
X. Zhang, G. Yuan, L. Mao, J. Niu, and W. Ding, “Biocorrosion properties of as-extruded Mg—Nd—Zn—Zr alloy compared with commercial AZ31 and WE43 alloys,” Mater. Lett., vol. 66, no. 1, pp. 209-211, 2012.
Z. Li, X. Gu, S. Lou, and Y. Zheng, “The development of binary Mg—Ca alloys for use as biodegradable materials within bone,” Biomaterials, vol. 29, no. 10, pp. 1329-1344, April 2008.
A. Krause, N. Von Der Höh, D. Bormann, C. Krause, F.-W. Bach, H. Windhagen, and A. Meyer-Lindenberg, “Degradation behaviour and mechanical properties of magnesium implants in rabbit tibiae,” J. Mater. Sci., vol. 45, no. 3, pp. 624-632, 2010.
J.-c. Gao, S. Wu, L.-y. Qiao, and Y. Wang, “Corrosion behavior of Mg and Mg—Zn alloys in simulated body fluid,” Trans. Nonferrous Met. Soc. China Engl. Ed., vol. 18, no. 3, pp. 588-592, 2008.
L. Yang, Y. Huang, F. Feyerabend, R. Willumeit, K. U. Kainer, and N. Hort, “Influence of ageing treatment on microstructure, mechanical and bio-corrosion properties of Mg—Dy alloys,” J. Mech. Behay. Biomed. Mater., vol. 13, pp. 36-44, September 2012.
S. Farè, Q. Ge, M. Vedani, G. V. D. Gastaldi, F. Migliavacca, and L. P. S. Trasatti, “Evaluation of material properties and design requirements for biodegradable magnesium stents,” Rev. Mater., vol. 15, no. 2, pp. 103-112, 2010.
L. Geris, H. van Oosterwyck, J. vander Sloten, J. Duyck, and I. Naert, “Assessment of mechanobiological models for the numerical simulation of tissue differentiation around immediately loaded implants,” Comput. Methods Biomech. Biomed. Engin., vol. 6, no. 5-6, pp. 277-288, 2003.
Y. Nakamura, Y. Tsumura, Y. Tonogai, T. Shibata, and Y. Ito, “Differences in behavior among the chlorides of seven rare earth elements administered intravenously to rats,” Fundam. Awl. Toxicol. Off. J. Soc. Toxicol., vol. 37, no. 2, pp. 106-116, June 1997.
Y. J. Chen, Y. J. Li, J. C. Walmsley, S. Dumoulin, P. C. Skaret, and H. J. Roven, “Microstructure evolution of commercial pure titanium during equal channel angular pressing,” Mater. Sci. Eng. A, vol. 527, no. 3, pp. 789-796, January 2010.
World Health Organization, Food and Agriculture Organization of the United Nations, and International Atomic Energy Agency, Trace elements in human nutrition and health. Geneva: World Health Organization, 1996.
N. T. Kirkland, M. P. Staiger, D. Nisbet, C. H. J. Davies, and N. Birbilis, “Performance-driven design of Biocompatible Mg alloys,” JOM, vol. 63, no. 6, pp. 28-34, June 2011.
J. Walker, S. Shadanbaz, T. B. F. Woodfield, M. P. Staiger, and G. J. Dias, “The in vitro and in vivo evaluation of the biocompatibility of Mg alloys,” Biomed. Mater., vol. 9, no. 1, p. 015006, February 2014.
X. N. Gu, X. L. Li, W. R. Zhou, Y. Cheng, and Y. F. Zheng, “Microstructure, biocorrosion and cytotoxicity evaluations of rapid solidified Mg-3Ca alloy ribbons as a biodegradable material,” Biomed. Mater., vol. 5, no. 3, p. 035013, June 2010.
B. Zhang, Y. Hou, X. Wang, Y. Wang, and L. Geng, “Mechanical properties, degradation performance and cytotoxicity of Mg—Zn—Ca biomedical alloys with different compositions,” Mater. Sci. Eng. C, vol. 31, no. 8, pp. 1667-1673, 2011.
R. M. Lozano, B. T. Pérez-Maceda, M. Carboneras, E. Onofre-Bustamante, M. C. Garcia-Alonso, and M. L. Escudero, “Response of MC3T3-E1 osteoblasts, L929 fibroblasts, and J774 macrophages to fluoride surface-modified AZ31 magnesium alloy,” J. Biomed. Mater. Res. A, vol. 101, no. 10, pp. 2753-2762, October 2013.
D. Hong, P. Saha, D.-T. Chou, B. Lee, B. E. Collins, Z. Tan, Z. Dong, and P. N. Kumta, “In vitro degradation and cytotoxicity response of Mg-4% Zn-0.5% Zr (ZK40) alloy as a potential biodegradable material,” Acta Biomater., vol. 9, no. 10, pp. 8534-8547, 2013.
X. N. Gu, N. Li, Y. F. Zheng, and L. Ruan, “In vitro degradation performance and biological response of a Mg—Zn—Zr alloy,” Mater. Sci. Eng. B, vol. 176, no. 20, pp. 1778-1784, December 2011.
D.-T. Chou, D. Hong, P. Saha, J. Ferrero, B. Lee, Z. Tan, Z. Dong, and P. N. Kumta, “In vitro and in vivo corrosion, cytocompatibility and mechanical properties of biodegradable Mg—Y—Ca—Zr alloys as implant materials,” Acta Biomater., vol. 9, no. 10, pp. 8518-8533, November 2013.
Z. Zhen, T. Xi, Y. Zheng, L. Li, and L. Li, “In Vitro Study on Mg—Sn—Mn Alloy as Biodegradable Metals,” J. Mater. Sci. Technol., vol. 30, no. 7, pp. 675-685, July 2014.
B. P. Zhang, Y. Wang, and L. Geng, “Research on Mg—Zn—Ca Alloy as Degradable Biomaterial,” in Biomaterials—Physics and Chemistry, R. Pignatello, Ed. InTech, 2011.
AAM I, ANSI/AAMI/ISO 10993-5:2009—Biological evaluation of medical devices—Part 5: Tests for in vitro cytotoxicity. AAMI, 2009.
M. M. Farag, Material Selection for Engineering Design, 1st edition. London; New York: Prentice Hall, 1997.
H. H. Bayraktar, E. F. Morgan, G. L. Niebur, G. E. Morris, E. K. Wong, and T. M. Keaveny, “Comparison of the elastic and yield properties of human femoral trabecular and cortical bone tissue,” J. Biomech., vol. 37, no. 1, pp. 27-35, January 2004.
M. A. Maleque and M. S. Salit, Materials Selection and Design. Springer Science & Business Media, 2013.
M. Kutz, Handbook of Materials Selection. John Wiley & Sons, 2002.
B. Dehghan-Manshadi, H. Mahmudi, A. Abedian, and R. Mahmudi, “A novel method for materials selection in mechanical design: Combination of non-linear normalization and a modified digital logic method,” Mater. Des., vol. 28, no. 1, pp. 8-15, 2007.
R. J. Mailloux, J. Lemire, and V. D. Appanna, “Hepatic response to aluminum toxicity: Dyslipidemia and liver diseases,” Exp. Cell Res., vol. 317, no. 16, pp. 2231-2238, October 2011.
J. D. Crowley, D. A. Traynor, and D. C. Weatherburn, “Enzymes and proteins containing manganese: an overview,” Met. Ions Biol. Syst., vol. 37, pp. 209-278, 2000.
T. M. Keaveny, E. F. Morgan, G. L. Niebur, and O. C. Yeh, Biomechanics of trabecular bone, vol. 3. 2001.
D. M. L. Cooper, J. R. Matyas, M. A. Katzenberg, and B. Hallgrimsson, “Comparison of microcomputed tomographic and microradiographic measurements of cortical bone porosity,” Calcif. Tissue Int., vol. 74, no. 5, pp. 437-447, May 2004.
C. A. Léon Y León, “New perspectives in mercury porosimetry,” Adv. Colloid Interface Sci., vol. 76-77, pp. 341-372, 1998.
S. Van Bael, Y. C. Chai, S. Truscello, M. Moesen, G. Kerckhofs, H. Van Oosterwyck, J.-P. Kruth, and J. Schrooten, “The effect of pore geometry on the in vitro biological behavior of human periosteum-derived cells seeded on selective laser-melted Ti6Al4V bone scaffolds,” Acta Biomater., vol. 8, no. 7, pp. 2824-2834, July 2012.
E. S. Gadelmawla, M. M. Koura, T. M. A. Maksoud, I. M. Elewa, and H. H. Soliman, “Roughness parameters,” J. Mater. Process. Technol., vol. 123, no. 1, pp. 133-145, April 2002.
“Roughness (2D) parameter I.” [Online]. Available: http://www.olympus-ims.com/en/knowledge/metrology/roughness/2d_parameter/. [Accessed: 10 Apr. 2015].
H. E. Friedrich and B. L. Mordike, Magnesium Technology: Metallurgy, Design Data, Automotive Applications. Springer Science & Business Media, 2006.
J. Wei, J. Jia, F. Wu, S. Wei, H. Zhou, H. Zhang, J.-W. Shin, and C. Liu, “Hierarchically microporous/macroporous scaffold of magnesium-calcium phosphate for bone tissue regeneration,” Biomaterials, vol. 31, no. 6, pp. 1260-1269, February 2010.
I. I. of Metals, Metals and Materials. Allied Publishers, 2011.
C. D. Yim, B. S. You, R. S. Jang, and S. G. Lim, “Effects of melt temperature and mold preheating temperature on the fluidity of Ca containing AZ31 alloys,” J. Mater. Sci., vol. 41, no. 8, pp. 2347-2350, March 2006.
B. Bhushan, Modern Tribology Handbook, Two Volume Set. CRC Press, 2000.
S. Banerjee, R. Yang, C. E. Courchene, and T. E. Conners, “Scanning Electron Microscopy Measurements of the Surface Roughness of Paper,” Ind. Eng. Chem. Res., vol. 48, no. 9, pp. 4322-4325, May 2009.
K. Anselme and M. Bigerelle, “On the relation between surface roughness of metallic substrates and adhesion of human primary bone cells,” Scanning, vol. 36, no. 1, pp. 11-20, 2014.
S. Puckett, R. Pareta, and T. J. Webster, “Nano rough micron patterned titanium for directing osteoblast morphology and adhesion,” Int. J. Nanomedicine, vol. 3, no. 2, pp. 229-241, June 2008.
W.-Y. Jang, S. Kyriakides, and A. M. Kraynik, “On the compressive strength of open-cell metal foams with Kelvin and random cell structures,” Int. J. Solids Struct., vol. 47, no. 21, pp. 2872-2883, October 2010.
L. J. Gibson and M. F. Ashby, Cellular Solids: Structure and Properties. Cambridge University Press, 1999.
A. Nouri, “Novel metal structures through powder metallurgy for biomedical applications,” 2008.
L. E. McNamara, R. J. McMurray, M. J. P. Biggs, F. Kantawong, R. O. C. Oreffo, and M. J. Dalby, “Nanotopographical Control of Stem Cell Differentiation,” J. Tissue Eng., vol. 2010, August 2010.
C. H. Thomas, J. H. Collier, C. S. Sfeir, and K. E. Healy, “Engineering gene expression and protein synthesis by modulation of nuclear shape,” Proc. Natl. Acad. Sci., vol. 99, no. 4, pp. 1972-1977, February 2002.
P. Ayala, J. I. Lopez, and T. A. Desai, “Microtopographical Cues in 3D Attenuate Fibrotic Phenotype and Extracellular Matrix Deposition: Implications for Tissue Regeneration,” Tissue Eng. Part A, vol. 16, no. 8, pp. 2519-2527, March 2010.
C. M. Kirschner, J. F. Schumacher, and A. B. Brennan, “Cellular Responses to Bio-Inspired Engineered Topography,” in Bio-inspired Materials for Biomedical Engineering, A. B. Brennan and C. M. Kirschner, Eds. John Wiley & Sons, Inc., 2014, pp. 77-97.
C. Liu, Y. Zhao, Y. Chen, P. Liu, and K. Cai, “Surface modification of magnesium alloy via cathodic plasma electrolysis and its influence on corrosion resistance and cytocompatibility,” Mater. Lett., vol. 132, pp. 15-18, October 2014.
B. Zberg, P. J. Uggowitzer, and J. F. Löffler, “MgZnCa glasses without clinically observable hydrogen evolution for biodegradable implants,” Nat. Mater., vol. 8, no. 11, pp. 887-891, 2009.
A. Dhanapal, S. Rajendra Boopathy, and V. Balasubramanian, “Corrosion behaviour of friction stir welded AZ61A magnesium alloy welds immersed in NaCl solutions,” Trans. Nonferrous Met. Soc. China, vol. 22, no. 4, pp. 793-802, April 2012.
X. Liu, P. K. Chu, and C. Ding, “Surface nano-functionalization of biomaterials,” Mater. Sci. Eng. R Rep., vol. 70, no. 3-6, pp. 275-302, November 2010.
J. Yang, F. Cui, I. S. Lee, and X. Wang, “Plasma surface modification of magnesium alloy for biomedical application,” Surf. Coat. Technol., vol. 205, Supplement 1, pp. S182-S187, December 2010.
G. Manivasagam and S. Suwas, “Biodegradable Mg and Mg based alloys for biomedical implants,” Mater. Sci. Technol. U. K., vol. 30, no. 5, pp. 515-520, 2014.
G. L. Song, Corrosion of Magnesium Alloys. Elsevier, 2011.
Y. Zhang, K. Bai, Z. Fu, C. Zhang, H. Zhou, L. Wang, S. Zhu, S. Guan, D. Li, and J. Hu, “Composite coating prepared by micro-arc oxidation followed by sol-gel process and in vitro degradation properties,” Appl. Surf. Sci., vol. 258, no. 7, pp. 2939-2943, January 2012.
J. H. Gao, S. K. Guan, J. Chen, L. G. Wang, S. J. Zhu, J. H. Hu, and Z. W. Ren, “Fabrication and characterization of rod-like nano-hydroxyapatite on MAO coating supported on Mg—Zn—Ca alloy,” Appl. Surf. Sci., vol. 257, no. 6, pp. 2231-2237, January 2011.
Y. Shi, M. Qi, Y. Chen, and P. Shi, “MAO-DCPD composite coating on Mg alloy for degradable implant applications,” Mater. Lett., vol. 65, no. 14, pp. 2201-2204, July 2011.
Y. Gu, C. Chen, S. Bandopadhyay, C. Ning, Y. Zhang, and Y. Guo, “Corrosion mechanism and model of pulsed DC microarc oxidation treated AZ31 alloy in simulated body fluid,” Appl. Surf. Sci., vol. 258, no. 16, pp. 6116-6126, June 2012.
X. N. Gu, N. Li, W. R. Zhou, Y. F. Zheng, X. Zhao, Q. Z. Cai, and L. Ruan, “Corrosion resistance and surface biocompatibility of a microarc oxidation coating on a Mg—Ca alloy,” Acta Biomater., vol. 7, no. 4, pp. 1880-1889, April 2011.
Y. Xin, C. Liu, K. Huo, G. Tang, X. Tian, and P. K. Chu, “Corrosion behavior of ZrN/Zr coated biomedical AZ91 magnesium alloy,” Surf. Coat. Technol., vol. 203, no. 17-18, pp. 2554-2557, June 2009.
Y. Zhang, J. X. Yang, F. Z. Cui, I.-S. Lee, and G.-H. Lee, “Characterization and degradation comparison of DLC film on different magnesium alloys,” Surf Coat. Technol., vol. 205, Supplement 1, pp. S15-S20, December 2010.
J. Hu, C. Zhang, B. Cui, K. Bai, S. Guan, L. Wang, and S. Zhu, “In vitro degradation of AZ31 magnesium alloy coated with nano TiO2 film by sol-gel method,” Appl. Surf. Sci., vol. 257, no. 21, pp. 8772-8777, August 2011.
X. Liu, P. K. Chu, and C. Ding, “Surface modification of titanium, titanium alloys, and related materials for biomedical applications,” Mater. Sci. Eng. R Rep., vol. 47, no. 3-4, pp. 49-121, December 2004.
R. Xu, G. Wu, X. Yang, T. Hu, Q. Lu, and P. K. Chu, “Controllable degradation of biomedical magnesium by chromium and oxygen dual ion implantation,” Mater. Lett., vol. 65, no. 14, pp. 2171-2173, July 2011.
G. Wu, R. Xu, K. Feng, S. Wu, Z. Wu, G. Sun, G. Zheng, G. Li, and P. K. Chu, “Retardation of surface corrosion of biodegradable magnesium-based materials by aluminum ion implantation,” Appl. Surf. Sci., vol. 258, no. 19, pp. 7651-7657, July 2012.
X. N. Gu, Y. F. Zheng, Q. X. Lan, Y. Cheng, Z. X. Zhang, T. F. Xi, and D. Y. Zhang, “Surface modification of an Mg-1Ca alloy to slow down its biocorrosion by chitosan,” Biomed. Mater., vol. 4, no. 4, p. 044109, August 2009.
L. Xu, E. Zhang, and K. Yang, “Phosphating treatment and corrosion properties of Mg—Mn—Zn alloy for biomedical application,” J. Mater. Sci. Mater. Med., vol. 20, no. 4, pp. 859-867, 2009.
L. Xu, F. Pan, G. Yu, L. Yang, E. Zhang, and K. Yang, “In vitro and in vivo evaluation of the surface bioactivity of a calcium phosphate coated magnesium alloy,” Biomaterials, vol. 30, no. 8, pp. 1512-1523, 2009.
Y. M. Wang, F. H. Wang, M. J. Xu, B. Zhao, L. X. Guo, and J. H. Ouyang, “Microstructure and corrosion behavior of coated AZ91 alloy by microarc oxidation for biomedical application,” Appl. Surf. Sci., vol. 255, no. 22, pp. 9124-9131, 2009.
F. Czerwinski, Ed., Magnesium Alloys—Corrosion and Surface Treatments. InTech, 2011.
H. Hornberger, S. Virtanen, and A. R. Boccaccini, “Biomedical coatings on magnesium alloys—A review,” Acta Biomater., vol. 8, no. 7, pp. 2442-2455, July 2012.
T. Ishizaki, J. Hieda, N. Saito, N. Saito, and O. Takai, “Corrosion resistance and chemical stability of super-hydrophobic film deposited on magnesium alloy AZ31 by microwave plasma-enhanced chemical vapor deposition,” Electrochimica Acta, vol. 55, no. 23, pp. 7094-7101, September 2010.
R. Gao, Q. Liu, J. Wang, X. Zhang, W. Yang, J. Liu, and L. Liu, “Fabrication of fibrous szaibelyite with hierarchical structure superhydrophobic coating on AZ31 magnesium alloy for corrosion protection,” Chem. Eng. J., vol. 241, pp. 352-359, April 2014.
Y. Wang, W. Wang, L. Zhong, J. Wang, Q. Jiang, and X. Guo, “Super-hydrophobic surface on pure magnesium substrate by wet chemical method,” Appl. Surf. Sci., vol. 256, no. 12, pp. 3837-3840, April 2010.
Q. Liu and Z. Kang, “One-step electrodeposition process to fabricate superhydrophobic surface with improved anticorrosion property on magnesium alloy,” Mater. Lett., vol. 137, pp. 210-213, December 2014.
Z. Wang, Q. Li, Z. She, F. Chen, and L. Li, “Low-cost and large-scale fabrication method for an environmentally-friendly superhydrophobic coating on magnesium alloy,” J. Mater. Chem., vol. 22, no. 9, pp. 4097-4105, February 2012.
Z. Zhen, T. XI, and Y. Zheng, “A review on in vitro corrosion performance test of biodegradable metallic materials,” Trans. Nonferrous Met. Soc. China, vol. 23, no. 8, pp. 2283-2293, August 2013.
T. Ishizaki, S. Lee, and K. Teshim, “High Functionalization of Magnesium Alloy Surface by Superhydrophobic Treatment,” in Magnesium Alloys—Corrosion and Surface Treatments, F. Czerwinski, Ed. InTech, 2011.
T. P. Kunzler, C. Huwiler, T. Drobek, J. Vörös, and N. D. Spencer, “Systematic study of osteoblast response to nanotopography by means of nanoparticle-density gradients,” Biomaterials, vol. 28, no. 33, pp. 5000-5006, November 2007.
A. Pierres, V. Monnet-Corti, A.-M. Benoliel, and P. Bongrand, “Do membrane undulations help cells probe the world?,” Trends Cell Biol., vol. 19, no. 9, pp. 428-433, September 2009.
A. Wennerberg and T. Albrektsson, “Effects of titanium surface topography on bone integration: a systematic review,” Clin. Oral Implants Res., vol. 20 Suppl 4, pp. 172-184, September 2009.
F. Song, Z. Li, X. Li, L. Zhang, M. Han, and G. Wang, “Ion sputtering nanostructuring crystalline MgF2 surface and its energy-dependent surface roughness,” Mod. Phys. Lett. B, vol. 19, no. 04, pp. 157-162, February 2005.
Y. Morimoto, Y. Tanaka, and A. Ide-Ektessabi, “Investigation of effects of ion beam irradiation on properties of magnesium oxide films,” in Symposium OO—Growth, Modification, and Analysis by Ion Beams at the Nanoscale, 2005, vol. 908.
M. B. Guseva, V. V. Khvostov, B. M. Kostishko, and S. J. Kovalenko, “Interaction of low-energy Ar+ ions with the surfaces of Al₂O₃ and MgO,” Surf Sci. Lett., vol. 276, no. 1-3, pp. L24-L27, October 1992.
Y. Morimoto, Y. Tanaka, and A. Ide-Ektessabi, “Low energy ion induced secondary electron emission coefficient of magnesium oxide films,” Nucl. Instrum. Methods Phys. Res. Sect. B Beam Interact. Mater. At., vol. 249, no. 1-2, pp. 440-443, August 2006.
P. Riccardi, M. Ishimoto, P. Barone, and R. A. Baragiola, “Ion-induced electron emission from MgO by exciton decay into vacuum,” Surf Sci., vol. 571, no. 1-3, pp. L305-L310, November 2004.
F. Gentile, L. Tirinato, E. Battista, F. Causa, C. Liberale, E. M. di Fabrizio, and P. Decuzzi, “Cells preferentially grow on rough substrates,” Biomaterials, vol. 31, no. 28, pp. 7205-7212, October 2010.
N. Yasui, H. Nomura, and A. Ide-Ektessabi, “Characteristics of ion beam modified magnesium oxide films,” Thin Solid Films, vol. 447-448, pp. 377-382, January 2004.
D.-I. Kim, J.-Y. Lim, Y.-G. Kim, J.-J. Ko, C.-W. Lee, G.-S. Cho, and E.-H. Choi, “Ion-Induced Secondary Electron Emission Coefficient (γ) of Bulk-MgO Single Crystals,” Jpn. J. Appl. Phys., vol. 39, no. 4R, p. 1890, April 2000.
D. Thirumalaikumarasamy, K. Shanmugam, and V. Balasubramanian, “Comparison of the corrosion behaviour of AZ31B magnesium alloy under immersion test and potentiodynamic polarization test in NaCl solution,” J. Magnes. Alloys, vol. 2, no. 1, pp. 36-49, March 2014.
S. Gonzlez, E. Pellicer, S. Suriach, M. D. Bar, and J. Sort, “Biodegradation and Mechanical Integrity of Magnesium and Magnesium Alloys Suitable for Implants,” in Biodegradation—Engineering and Technology, R. Chamy, Ed. InTech, 2013.
F. Liu, Y. Song, D. Shan, and E.-H. Han, “Corrosion Mechanism of AM50 Magnesium Alloy in Simulated Acid Rain,” Recent Pat. Corros. Sci., vol. 3, no. 1, pp. 47-57, September 2013.
R. Ambat, N. N. Aung, and W. Zhou, “Evaluation of microstructural effects on corrosion behaviour of AZ91D magnesium alloy,” Corros. Sci., vol. 42, no. 8, pp. 1433-1455, August 2000.
D. C. Montgomery, Design and Analysis of Experiments, 8 edition. Hoboken, N.J.: Wiley, 2012.
W. Strober, “Trypan Blue Exclusion Test of Cell Viability,” in Current Protocols in Immunology, John Wiley & Sons, Inc., 2001.
I. Yamada, H. Ishiwara, and E. Kamijo, Laser and Ion Beam Modification of Materials: Proceedings of the Symposium U: Material Synthesis and Modification by Ion Beams and Laser Beams of the 3rd IUMRS International Conference on Advanced Materials, Sunshine City, Ikebukuro, Tokyo, Japan, August 31-Sep. 4, 1993. Elsevier, 2013.
“Introduction,” in Low-Energy Ion Irradiation of Solid Surfaces, Springer Berlin Heidelberg, 1999, pp. 1-6.
A. V. Krasheninnikov and F. Banhart, “Engineering of nanostructured carbon materials with electron or ion beams,” Nat. Mater., vol. 6, no. 10, pp. 723-733, 2007.
“Ionization processes of sputtered atoms and molecules,” in Low-Energy Ion Irradiation of Solid Surfaces, Springer Berlin Heidelberg, 1999, pp. 205-250.
M. Yazdimamaghani, M. Razavi, D. Vashaee, and L. Tayebi, “Surface modification of biodegradable porous Mg bone scaffold using polycaprolactone/bioactive glass composite,” Mater. Sci. Eng. C Mater. Biol. Appl., vol. 49, pp. 436-444, April 2015.
A. Yamamoto and S. Hiromoto, “Effect of inorganic salts, amino acids and proteins on the degradation of pure magnesium in vitro,” Mater. Sci. Eng. C, vol. 29, no. 5, pp. 1559-1568, June 2009.
W.-D. Mueller, M. F. L. de Mele, M. L. Nascimento, and M. Zeddies, “Degradation of magnesium and its alloys: dependence on the composition of the synthetic biological media,” J. Biomed. Mater. Res. A, vol. 90, no. 2, pp. 487-495, August 2009.
J. Fischer, D. Pröfrock, N. Hort, R. Willumeit, and F. Feyerabend, “Improved cytotoxicity testing of magnesium materials,” Mater. Sci. Eng. B Solid-State Mater. Adv. Technol., vol. 176, no. 11, pp. 830-834, 2011.
Life Technologies Corporation, “Medium 231 and SMGS.” [Online]. Available: http://tools.lifetechnologies.com/content/sfs/manuals/M231_SMGS_man.pdf. [Accessed: 14 Apr. 2015].
M. C. Merino, A. Pardo, R. Arrabal, S. Merino, P. Casajús, and M. Mohedano, “Influence of chloride ion concentration and temperature on the corrosion of Mg—Al alloys in salt fog,” Corros. Sci., vol. 52, no. 5, pp. 1696-1704, May 2010.
S. Bender, J. Goellner, A. Heyn, and E. Boese, “Corrosion and corrosion testing of magnesium alloys,” Mater. Corros., vol. 58, no. 12, pp. 977-982, December 2007.
T. L. Nguyen, A. Blanquet, M. P. Staiger, G. J. Dias, and T. B. F. Woodfield, “On the role of surface roughness in the corrosion of pure magnesium in vitro,” J. Biomed. Mater. Res. B Appl. Biomater, vol. 100, no. 5, pp. 1310-1318, July 2012.
R. Xin, B. Li, L. Li, and Q. Liu, “Influence of texture on corrosion rate of AZ31 Mg alloy in 3.5 wt. % NaCl,” Mater. Des., vol. 32, no. 8-9, pp. 4548-4552, September 2011.
R. Walter and M. B. Kannan, “Influence of surface roughness on the corrosion behaviour of magnesium alloy,” Mater. Des., vol. 32, no. 4, pp. 2350-2354, April 2011.
J. E. Schaffer, E. A. Nauman, and L. A. Stanciu, “Cold drawn bioabsorbable ferrous and ferrous composite wires: An evaluation of in vitro vascular cytocompatibility,” Acta Biomater., vol. 9, no. 10, pp. 8574-8584, 2013.
M. Li, L. Ren, L. Li, P. He, G. Lan, Y. Zhang, and K. Yang, “Cytotoxic Effect on Osteosarcoma MG-63 Cells by Degradation of Magnesium,” J. Mater. Sci. Technol., vol. 30, no. 9, pp. 888-893, September 2014.
J. A. M. Maier, A. Nasulewicz-Goldeman, M. Simonacci, A. Boninsegna, A. Mazur, and F. I. Wolf, “Insights into the mechanisms involved in magnesium-dependent inhibition of primary tumor growth,” Nutr. Cancer, vol. 59, no. 2, pp. 192-198, 2007.
Y. Zhang, L. Ren, M. Li, X. Lin, H. Zhao, and K. Yang, “Preliminary Study on Cytotoxic Effect of Biodegradation of Magnesium on Cancer Cells,” J. Mater. Sci. Technol., vol. 28, no. 9, pp. 769-772, 2012.
A. B. Brennan and C. M. Kirschner, Eds., Bio-inspired Materials for Biomedical Engineering, 1 edition. Wiley, 2014.

### Example 3—Synthesis of Mg-Based Porous Material

### Abstract

During the last decade, great interest in porous scaffold substitutes has emerged because of their applicability in bone tissue engineering. Porous material provides the necessary support for cells to grow, proliferate, and preserve their differentiated function. Hence, the shape and the properties of the new tissue are defined by the scaffold geometry. The main purpose of the present work is to build up the fabrication procedure to obtain porous Mg-based scaffolds.

For this, open cell magnesium foams are fabricated by replication process. The starting material is a commercially available ingot of AZ31. To prepare the preforms, NaCl grains are sieved in sizes of 500 μm and 1200 μm. The metal pieces are put onto the salt preform and covered by fluxes. No gas controlled atmosphere is used. Finally, the liquid metal is infiltrated into the preform by applying vacuum. At the point of solidification, the salt preforms are dissolved in water and the spaces of the original preform are mostly copied.

The results indicate that the porous Mg-based specimens with pore sizes replicated from the salt particles can have morphological properties comparable with those of cancellous bone. Furthermore, according to the results, the porous AZ31 alloy may be used as scaffold material for hard tissue regeneration.

### Introduction

Bone is the second most frequently transplanted tissue. Different from other tissues, bone can be regenerated and healed by itself. However, in intense fractures or large bone defects, the self-repair ability of the bone can fail resulting in slowed unions or non-unions. A bone scaffold is defined as an implanted material that promotes bone healing through osteogenesis, osteoinduction, and osteoconduction. This is a relatively complex device that should be degradable, allow cell attachment and provide mechanical support in order to avoid bone repair failure.

During the last decade, great interest in porous scaffold substitutes has emerged because of their applicability in bone tissue engineering. A porous material provides the necessary support for cells to grow, proliferate, and preserve their differentiated function. Hence, the shape and the properties of the new tissue are defined by the scaffold geometry.

The architectural bone scaffold parameters determine the biological outcome. The geometry of porous scaffolds in the case of bone substitution has been showing significant influence in the cellular response and the rate of bone tissue regeneration. In order to complete the vascularization of scaffolds, material chemistry and macro and micro-structural properties ought to be optimized. The rate of vascularization depends on pore size, porosity, pore interconnectivity, and volume ratio. A large surface area can lead to improved cell attachment, whereas highly porous scaffolds favor vascularization and nutrient interchange. Therefore, scaffolds for osteogenesis should mimic bone architecture, structure and function in order to enhance integration into surrounding tissues. The minimum pore size required to regenerate mineralized bone is considered to be 100 μm, smaller pores result in ingrowth of unmineralized tissue, and even smaller ones are penetrated only by fibrous tissue. Appropriate pore sizes for bone tissue engineering are reported to be >200 μm while rapid vascularization of matrices apparently requires a pore size greater than 500 μm. Hence, the general pore size for bone with high vascularization potential should be between 200 and 900 μm.

Replication process is a manufacturing method which consists of processing a preform that can be a sintered salt or a polymer foam impregnated with plaster slurry. The preform is then permeated with liquid metal, which infiltrates the free spaces between the grains. At the point of solidification the salt preform is dissolved in water and the spaces of the original preform are mostly copied. This process results in an open-cell almost fully interconnected foam. In the case of the polymer foam, the template is filled with a slurry of a more heat resistant material. After that, the polymer is removed by heat and liquid metal is casted into the cavities which reproduce the original template structure.

In general, the fabrication of porous structures based on Mg alloys has been limited, mainly because of the high reactive potential of this material, and consequently, the complications with the sintering. The processes described in the literature require modification to the equipment and the procedures that were already standardized for other metals. Since Mg is high flammable, material processing is normally performed in vacuum or inert gas environment.

### Experimental Methods

### Infiltration Casting

The starting material is a commercially available ingot of AZ31 Mg-based alloy. Then, foams are manufactured using a preform of salt particles sieved and separated in two average sizes, 500 μm and 1200 μm. NaCl particles are deposited into a refractory and the metal pieces were placed over salt the preform. The Mg-based alloy is protected from oxidation by the use of salt fluxes instead for controlled atmosphere with gases. The temperature is slowly increased from room temperature to around 680° C., then, it is maintained for the whole process. Then, vacuum is applied to create a negative pressure inside the chamber. The obtained structure is cut in several cylindrical samples that were leached by dissolution of the salt in water in order to reveal the open cells and the interconnected network of metal matrix. A total of eight assays were performed.

### Porosity Evaluation

Scanning electron microscope (JEOL 6060LV General Purpose SEM) is used to characterize the morphology of the pores. Then, the samples are evaluated by X-ray μCT. The X-ray power is set to the maximum of 8 Watts and 60 keV.

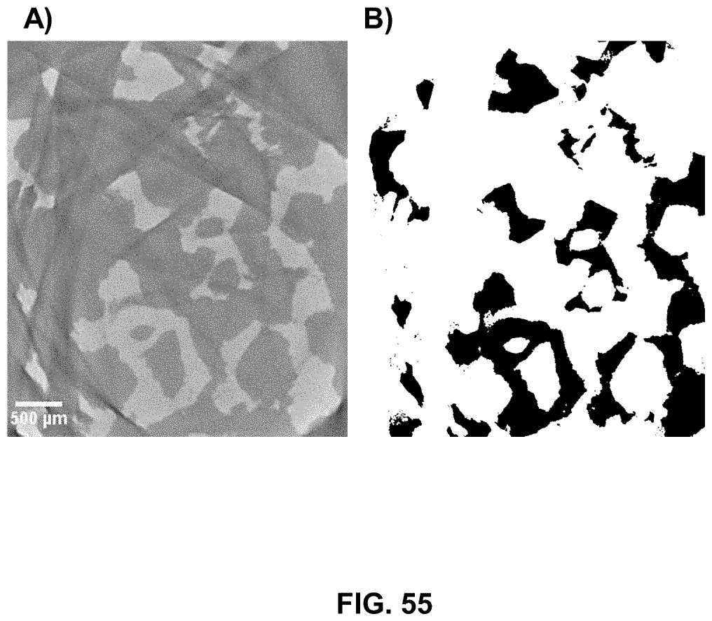

The percent porosity in gray scale is estimated from X-ray μCT slides by converting the original slide to a binary image and getting a numerical calculation of the area covered by pores vs. the area occupied by material. ImageJ software is used for this purpose (see FIG. 55). This analysis was repeated three times for each image and 10 slides were evaluated.

### Chemical and Crystallographic Analysis

Irregular samples are cut directly from two Mg-based foams. The chemical composition is assessed by EPA test methods for evaluating solid waste. The crystalline phase of the samples are studied by the X-ray diffraction (Philips X'pert MRD system) with a Cu Kα source.

### Compression Test

Uniaxial compression tests are carried out in a universal testing machine INSTRON, model 3366 at a crosshead speed of 2 mm/min. Two specimens for each condition are evaluated. Test samples are cut to 2.5 mm in diameter and 2 mm in height.

### Results

### Infiltration Casting

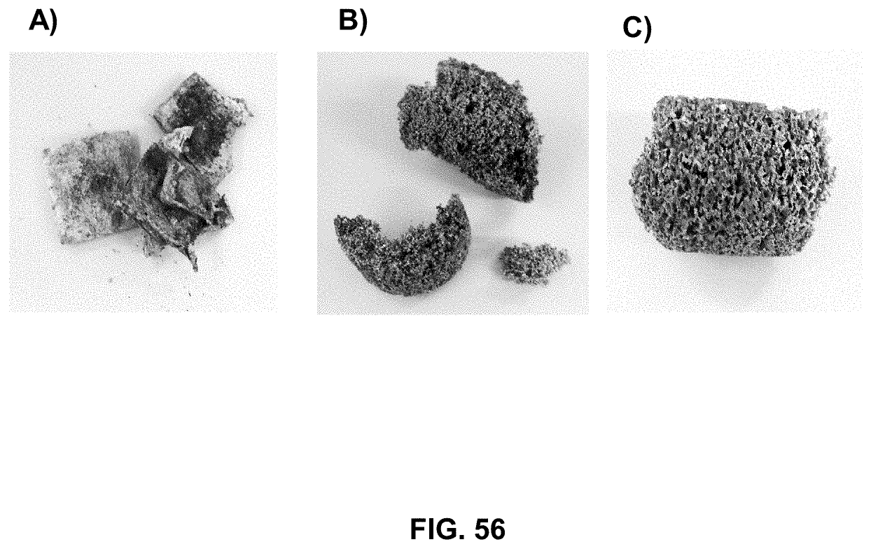

In terms of fluidity and the alloy ability to infiltrate the salt preform, three different results were observed among the obtained foams shown in FIG. 56: A) no infiltration of salt mold, B) acceptable foam with bulks of salt without infiltration, C) correct Mg-based alloy infiltration of the salt.

The more suitable parameters are melting temperature of 670° C. and melting time of 60 mins for infiltrating 1200 μm salt preform, and 680° C. and 100 min for infiltrating 500 μm salt preform. For the purpose of example, EMg1 and EMg2 nomenclature will be used for 500 μm foam and 1200 μm respectively.

### Porosity Evaluation

Since the metal foam samples are Mg-based alloy, the images obtained presented high contrast between the metal (1.77 g/cm³) and the air (0.00119 g/cm³). FIG. 55 shows a 2D slice from X-ray μCT in the dimension scale of 500 μm. The dark gray sections are pores, while the bright gray represents solid, most of which is Mg according to the X-ray diffraction (XRD) analysis.

In CT practice, the proper grayscale value for defining a boundary between two phases is the average of their two mean end-member grayscales. Therefore, the threshold gray scale was set as 150 for Mg and MgO area. Average porosities were determinate as 87.19% for EMg2, and 71.35% for EMg1, by using ImageJ.

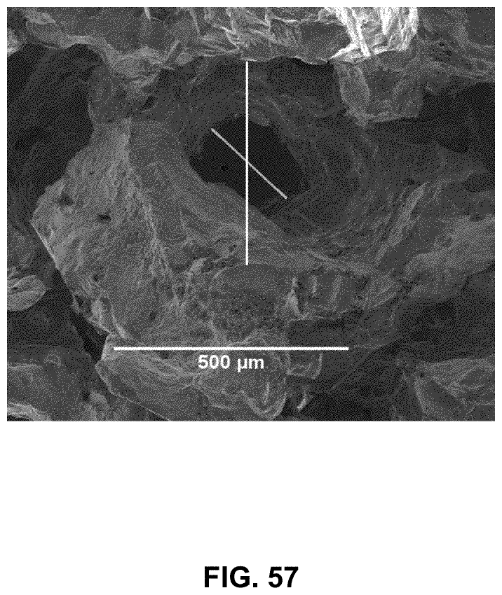

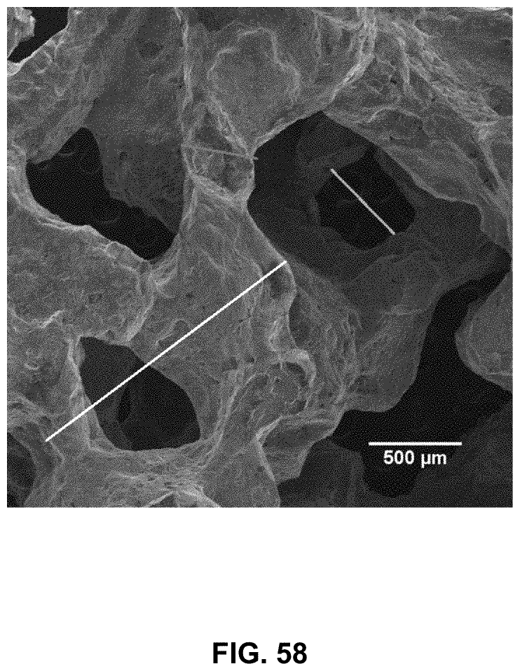

The two foam samples fabricated from the same ingot have similar porous structure with different pore sizes, showing nearly isotropic characteristics in the surface as is showed in FIGS. 57 and 58. The foams contain mainly two types of pores: the macropores (white line) acquired as a result of the dissolution of the salt grains and the small pores (cyan line) derivate from the interparticle contacts. The small pores (−210 μm for EMg1 and ˜435 μm for EMg2) are usually distributed on the cell center, generating the connection tunnels that made the foams present very high open porosity (71.35% and 87.19%). The cell wall thickness (red line) is in the range of 90 μm and the cell edge (green line) has nonuniformity in dimension.

The size of the macropores, ˜497 μm for EMg1 and ˜1300 μm for EMg2, was mainly determined by the infiltration pressure, the salt particle dimensions and the casting temperature when the density of Mg-based alloy is constant. Thus, it could be deduced that the infiltration parameters play a role on the connectivity of Mg-based alloy foams, which would also provide great flexibility for the structural design of ellipsoidal pores Mg-based foams.

### Chemical Evaluation

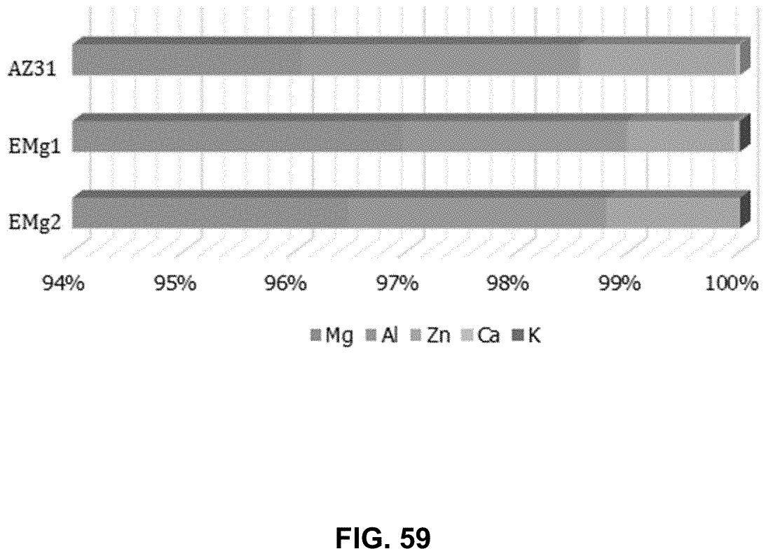

EPA test methods for evaluating solid waste is used to investigate the levels of Al, Ca, Mg, K, and Zn extracted from EMg2 and EMg1. In both cases, the levels for all metals are hierarchized as follows: Mg>>Al>Zn>>Ca>K. The metal concentration levels in EMg2 and EMg1 are shown in FIG. 59. The results reveal only minor discrepancies between porous samples composition and AZ31 alloy composition [21], except for the apparition of an small K trace in the EMg2 and EMg1 samples.

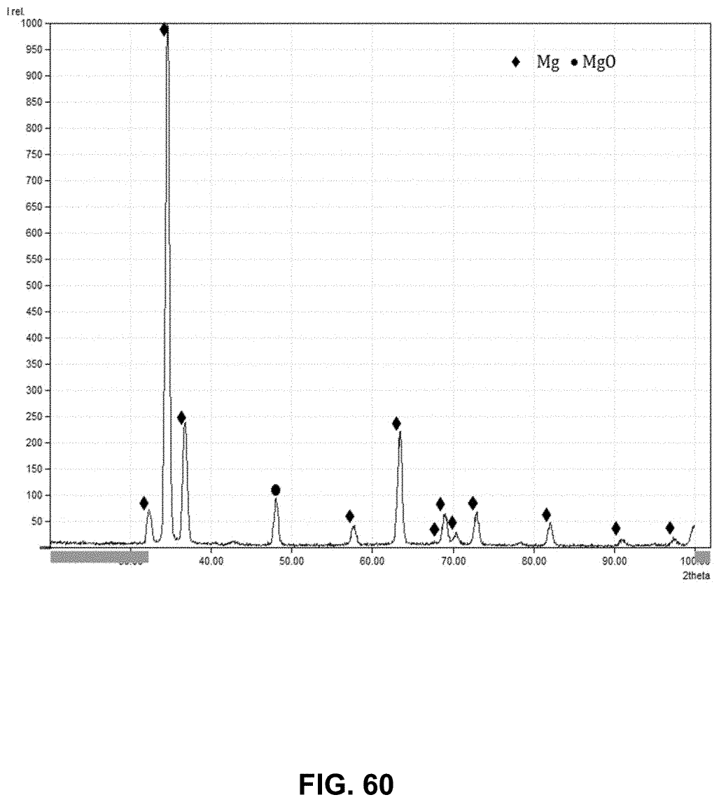

The crystalline phase of the surfaces and original material was studied by XRD. FIG. 60 shows the XRD patterns of the original ingot. This material mainly contains two kinds of peaks: Mg and MgO. No other phases were identified within the sensitivity limits of XRD. Zn and Al elements might not be detected because only small amounts of them are present on the alloy.

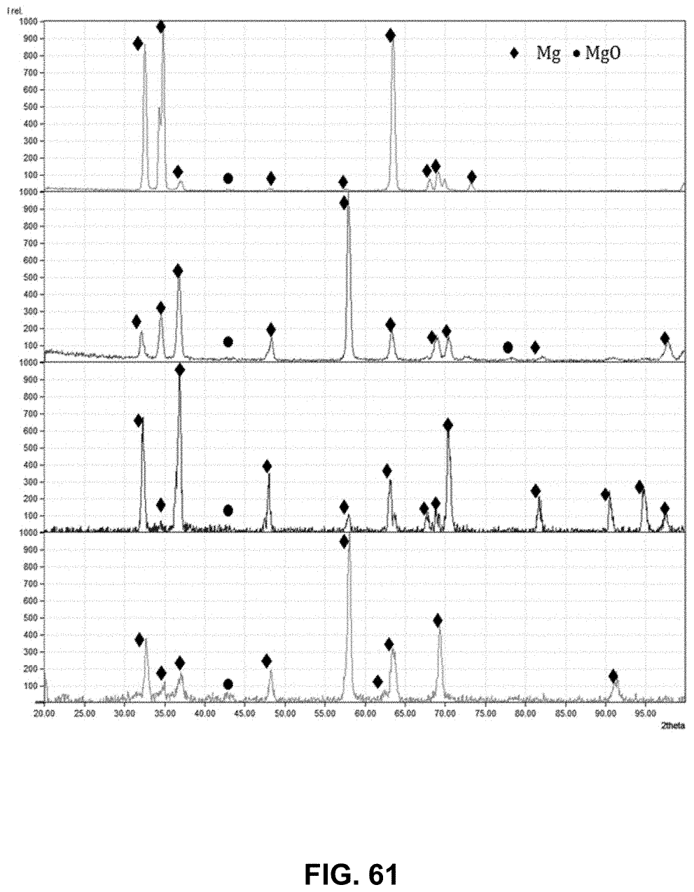

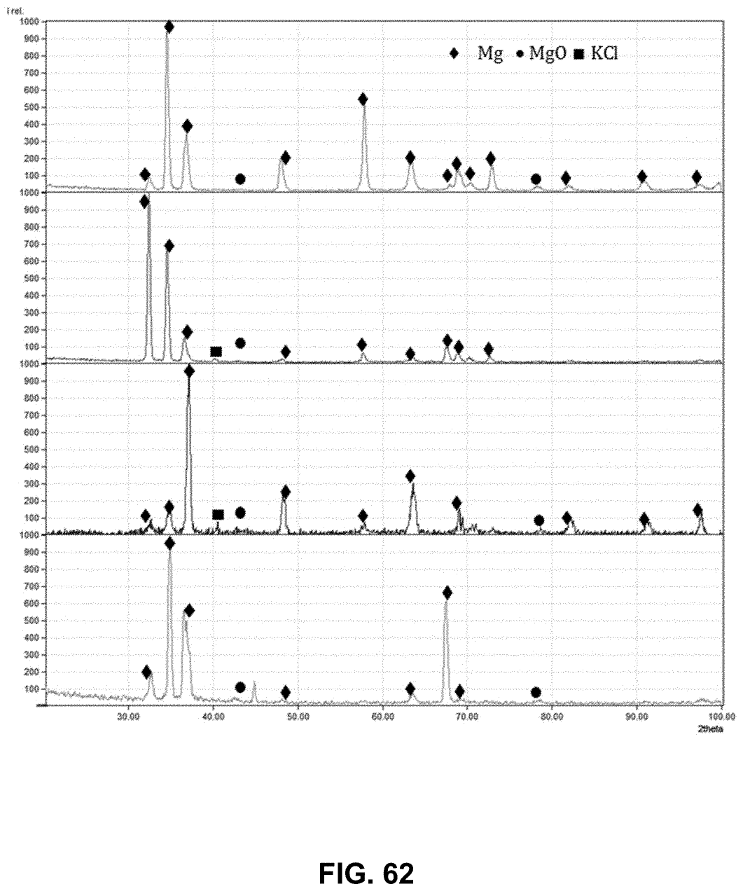

The peaks that appeared at 2e=42.9° and 78.4° respectively, represent (2 0 0) and (2 2 2) planes of the cubic MgO crystal structure. FIG. 61 shows that the phase compositions of the Mg-based foams are very similar to the original ingot (FIG. 60) and same peaks of MgO can be identified. FIG. 62 shows a comparable XRD pattern. However, MgO peaks were almost completely disappeared in samples EMg1a and EMg1b. There is also a peak at 2e=42.22° that turns up in samples EMg1c and EMg1d this peaks represents KCl, which was one of the salt fluxes used during the foams infiltration process. In all the cases, original ingot and obtained foams, Mg peaks were shifted to lower 2e values which could correspond to the dissolution of the larger Al and Zn atoms into the Mg matrix.

### Compression Test

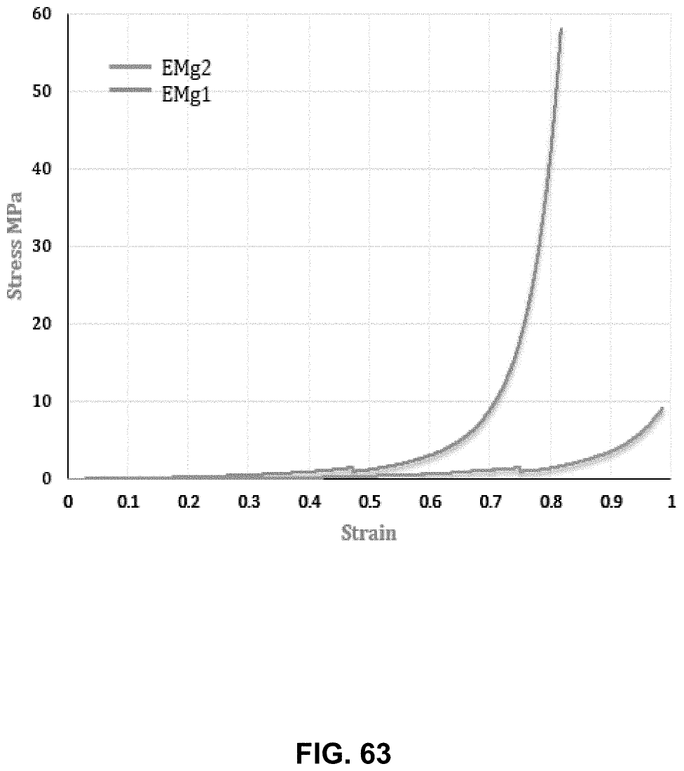

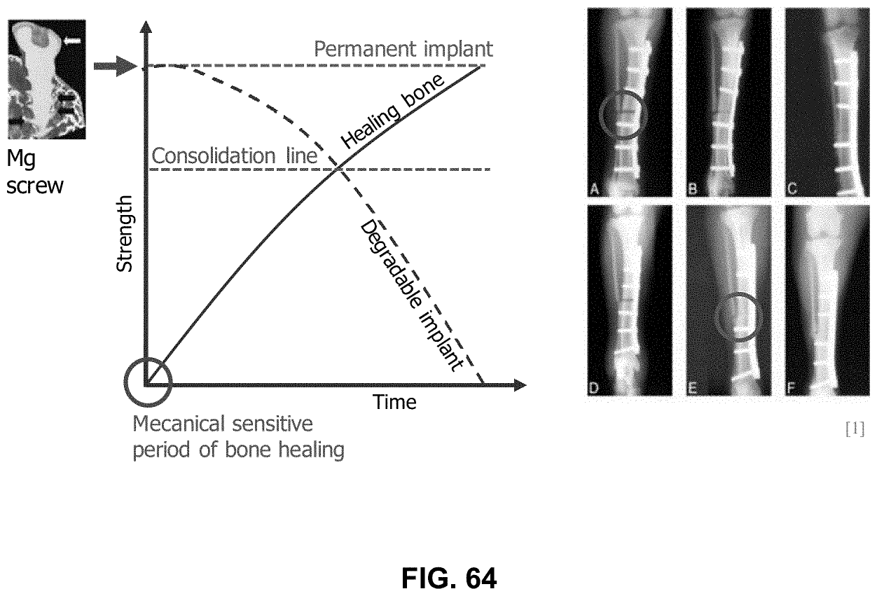

The effects of density and pore size on the mechanical properties of the Mg-based foams were examined by compressive test, as shown in FIG. 63. The curves exhibit a quite long plateau with a nearly constant stress level where the stress increases slowly as the cells deform plastically. This region ends at strain as high as 65-75% for EMg1 and 80-85% for EMg2. Later, there is a densification stage where the collapsed cells are compacted together and stress is rapidly increased.

The plateau regions are smooth, suggesting that foams are not brittle. This smooth plateau reflects the compaction deformation mode. As the compression proceeds, the foam is being quickly compacted, therefore, it can be seen at 48% strain for EMg1 and 75% for EMg2 where the foams are compacted and a change in the foams stiffness is notable.

### Discussion

In general, the fabrication of porous structures based on Mg alloys has been limited, mainly because of the high reactive potential of this material, and consequently, the complications with the sintering. In the actual state of the literature review in this work, all the processes described require modification to the equipment and the procedures that were already standardized for other metals. Therefore, casting fillings is inherently difficult due to low metallostatic pressure and rapid alloy solidification. Since Mg is high flammable, processing the material is normally performed in vacuum or inert gas environment. Hence, this work focuses its efforts on developing a reliable, safe and low cost method for manufacturing porous Mg foams.

Fluidity was one of the important steps in the manufacturing process since a high fluidity level, or an extended holding time would result in an over-infiltrated NaCl template. Yim et al. found an increment of fluidity of AZ31 with an increase in temperature and melting time. Moreover, oxide films on the melt surface can significantly raise the surface tension and viscosity of the molten metal and reduce its ability to fill the mold. Mechanical destruction was used to break down the oxide layers to achieve the desired fluidity values to fill the salt cavities.

The cover fluxes melted over the surface of the molten alloy, formed a protective coating which inhibits the molten metal from contacting ambient gases. Moreover, salt fluxes are suspected to attract and wet impurities in the Mg melt, resulting in their subtraction. Furthermore, in spite of no protective atmosphere, chemical analysis suggests that no chemical reaction occurred during the infiltration process, showing a very promising application prospect as biodegradable bone implant material. The amount of MgO observed on the pore wall FIGS. 61 and 62, was probably due to the surface oxidation of the foams stored at room temperature and also oxidation occurred during salt grains dissolution.

A typical compressive stress-strain curve for metal scaffolds with high porosity shows three differentiated behaviors. Firstly, there is a linear-elastic region which is characterized by an initial increase in stress. This initial high slope is associated with the stiffness of the porous samples. Subsequently, due to the collapse of the pores, the flow stress no longer increases with strain and there appears a wide stress plateau known as plateau or collapse region. The compression curves obtained in this work began with the plateau region and the regions are smooth, without the presence of serrations that are typically observed in other open-cell Mg-based foams [. This result suggests that foams are not brittle. The increase of plateau stresses is very slow and the densification did not begin until about a strain of 65% or over was reached. This means that the present Mg-based foam could be good in impact absorption applications.

### Conclusion

Described herein is the fabrication and characterization of Mg-based foams by infiltration casting suitable for manufacturing Mg-based foams for bone tissue engineering. Foams with two different porosities and pores size were obtained. For the infiltration process, the temperature and melting time have been defined as being 680° C. and 670° C. and 100 min and 60 min for infiltrating 1200 μm and 500 μm salt preform respectively. The precise selection depends on the pore size and porosity desired.

The result of the crystalline phase and chemical analysis further indicate a safe and biologically inert process. This avoids the problem of toxic solvents or materials and hold significant promise for biomedical application as bone interfacing implants. The foams also presented enough mechanical properties to fulfill the required mechanical response of some scaffold materials, depending on the porosities.

### REFERENCES

[1] S. K. Nandi, S. Roy, P. Mukherjee, B. Kundu, D. K. De, and D. Basu, “Orthopaedic applications of bone graft & graft substitutes: a review,” Indian J. Med. Res., vol. 132, pp. 15-30, July 2010.
[2] M. Scaglione, L. Fabbri, D. Dell′Omo, F. Gambini, and G. Guido, “Long bone nonunions treated with autologous concentrated bone marrow-derived cells combined with dried bone allograft,” Musculoskelet. Surg., vol. 98, no. 2, pp. 101-106, August 2014.
[3] A. Oryan, S. Alidadi, A. Moshiri, and N. Maffulli, “Bone regenerative medicine: classic options, novel strategies, and future directions,” J. Orthop. Surg., vol. 9, no. 1, p. 18, March 2014.
[4] I. Manjubala, A. Woesz, C. Pilz, M. Rumpler, N. Fratzl-Zelman, P. Roschger, J. Stampfl, and P. Fratzl, “Biomimetic mineral-organic composite scaffolds with controlled internal architecture,” J. Mater. Sci. Mater. Med., vol. 16, no. 12, pp. 1111-1119, December 2005.
[5] A. A. Zadpoor, “Bone tissue regeneration: the role of scaffold geometry,” Biomater Sci., vol. 3, no. 2, pp. 231-245, January 2015.
[6] S. Van Bael, Y. C. Chai, S. Truscello, M. Moesen, G. Kerckhofs, H. Van Oosterwyck, J.-P. Kruth, and J. Schrooten, “The effect of pore geometry on the in vitro biological behavior of human periosteum-derived cells seeded on selective laser-melted Ti6Al4V bone scaffolds,” Acta Biomater., vol. 8, no. 7, pp. 2824-2834, July 2012.
[7] A. R. Boccaccini, U. Kneser, and A. Arkudas, “Scaffolds for vascularized bone regeneration: advances and challenges,” Expert Rev. Med. Devices, vol. 9, no. 5, pp. 457-460, September 2012.
[8] S. Yang, K. F. Leong, Z. Du, and C. K. Chua, “The design of scaffolds for use in tissue engineering. Part I. Traditional factors,” Tissue Eng., vol. 7, no. 6, pp. 679-689, December 2001.
[9] V. Karageorgiou and D. Kaplan, “Porosity of 3D biomaterial scaffolds and osteogenesis,” Biomaterials, vol. 26, no. 27, pp. 5474-5491, September 2005.
[10] M. Chatzinikolaidou, S. Rekstyte, P. Danilevicius, C. Pontikoglou, H. Papadaki, M. Farsari, and M. Vamvakaki, “Adhesion and growth of human bone marrow mesenchymal stem cells on precise-geometry 3D organic-inorganic composite scaffolds for bone repair,” Mater. Sci. Eng. C, vol. 48, pp. 301-309, March 2015.
[11] P. Kasten, I. Beyen, P. Niemeyer, R. Luginbühl, M. Bohner, and W. Richter, “Porosity and pore size of β-tricalcium phosphate scaffold can influence protein production and osteogenic differentiation of human mesenchymal stem cells: An in vitro and in vivo study,” Acta Biomater., vol. 4, no. 6, pp. 1904-1915, November 2008.
[12] D. Logeart-Avramoglou, F. Anagnostou, R. Bizios, and H. Petite, “Engineering bone: challenges and obstacles,” J. Cell. Mol. Med., vol. 9, no. 1, pp. 72-84, March 2005.
[13] K. Kim, D. Dean, J. Wallace, R. Breithaupt, A. G. Mikos, and J. P. Fisher, “The influence of stereolithographic scaffold architecture and composition on osteogenic signal expression with rat bone marrow stromal cells,” Biomaterials, vol. 32, no. 15, pp. 3750-3763, May 2011.
[14] L. J. C. P. Fernandez, “Procesos de fabricación de metales celulares. Parte I: Procesos por via liquida,” Rev. Metal., 2008.
[15] J. Banhart, “Manufacture, characterisation and application of cellular metals and metal foams,” Prog. Mater. Sci., vol. 46, no. 6, pp. 559-632, 2001.
[16] H. Zhuang, Y. Han, and A. Feng, “Preparation, mechanical properties and in vitro biodegradation of porous magnesium scaffolds,” Mater. Sci. Eng. C, vol. 28, no. 8, pp. 1462-1466, December 2008.
[17] G. Hao, F. Han, and W. Li, “Processing and mechanical properties of magnesium foams,” J. Porous Mater., vol. 16, no. 3, pp. 251-256, June 2009.
[18] T. L. Nguyen, M. P. Staiger, G. J. Dias, and T. B. F. Woodfield, “A Novel Manufacturing Route for Fabrication of Topologically-Ordered Porous Magnesium Scaffolds,” Adv. Eng. Mater., vol. 13, no. 9, pp. 872-881, 2011.
[19] Z. S. Seyedraoufi and S. Mirdamadi, “Synthesis, microstructure and mechanical properties of porous MgIn scaffolds,” J. Mech. Behay. Biomed. Mater., vol. 21, pp. 1-8, May 2013.
[20] E07 Committee, “Guide for Computed Tomography (CT) Imaging,” ASTM International, 2011.
[21] H. E. Friedrich and B. L. Mordike, Magnesium Technology: Metallurgy, Design Data, Automotive Applications. Springer Science & Business Media, 2006.
[22] H. Zhuang, Y. Han, and A. Feng, “Preparation, mechanical properties and in vitro biodegradation of porous magnesium scaffolds,” Mater. Sci. Eng. C, vol. 28, no. 8, pp. 1462-1466, December 2008.
[23] J. Trinidad, I. Marco, G. Arruebarrena, J. Wendt, D. Letzig, E. Saenz de Argandona, and R. Goodall, “Processing of Magnesium Porous Structures by Infiltration Casting for Biomedical Applications: Processing of Magnesium Porous Structures by . . . ,” Adv. Eng. Mater., vol. 16, no. 2, pp. 241-247, February 2014.
[24] G. Jiang and G. He, “A new approach to the fabrication of porous magnesium with well-controlled 3D pore structure for orthopedic applications,” Mater. Sci. Eng. C Mater. Biol. Appl., vol. 43, pp. 317-320, October 2014.
[25] J. O. Osorio-Hernandez, M. A. Suarez, R. Goodall, G. A. Lara-Rodriguez, I. Alfonso, and I. A. Figueroa, “Manufacturing of open-cell Mg foams by replication process and mechanical properties,” Mater. Des., vol. 64, pp. 136-141, December 2014.
[26] P. Fernandez, L. J. Cruz, and J. Coleto, “Procesos de fabricación de metales celulares. Parte II: Via sólida, deposición de metales, otros procesos,” Rev. Metal., vol. 45, no. 2, pp. 124-142, April 2009.
[27] I. I. of Metals, Metals and Materials. Allied Publishers, 2011.
[28] C. D. Yim, B. S. You, R. S. Jang, and S. G. Lim, “Effects of melt temperature and mold preheating temperature on the fluidity of Ca containing AZ31 alloys,” J. Mater. Sci., vol. 41, no. 8, pp. 2347-2350, March 2006.
[29] W.-Y. Jang, S. Kyriakides, and A. M. Kraynik, “On the compressive strength of open-cell metal foams with Kelvin and random cell structures,” Int. J. Solids Struct., vol. 47, no. 21, pp. 2872-2883, October 2010.
[30] L. J. Gibson and M. F. Ashby, Cellular Solids: Structure and Properties. Cambridge University Press, 1999.
[31] X. Wang, Z. Li, Y. Huang, K. Wang, X. Wang, and F. Han, “Processing of magnesium foams by weakly corrosive and highly flexible space holder materials,” Mater. Des., vol. 64, pp. 324-329, December 2014.

### Example 4—Preferential Increase of Aluminum Concentration at Modified Surface

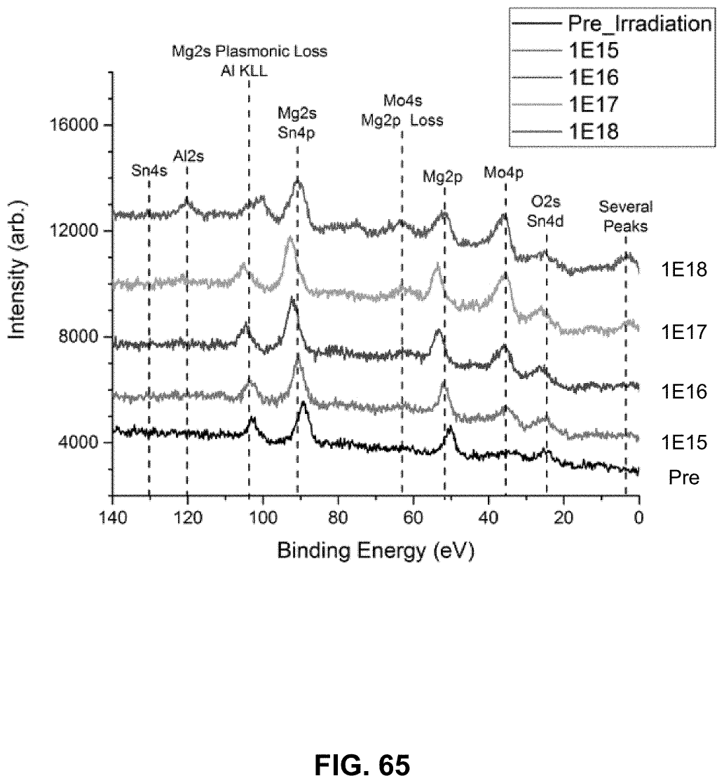

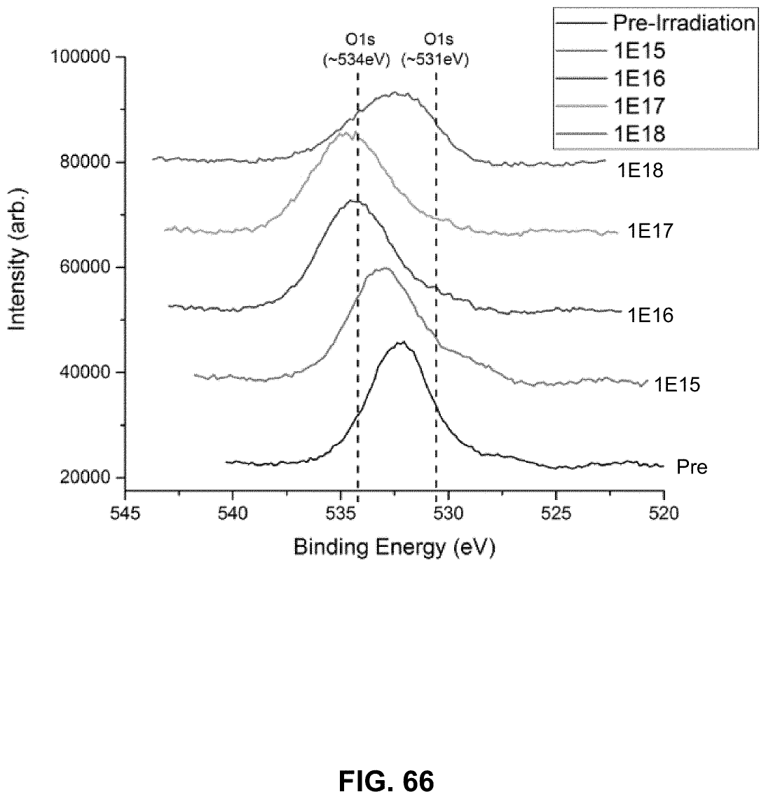

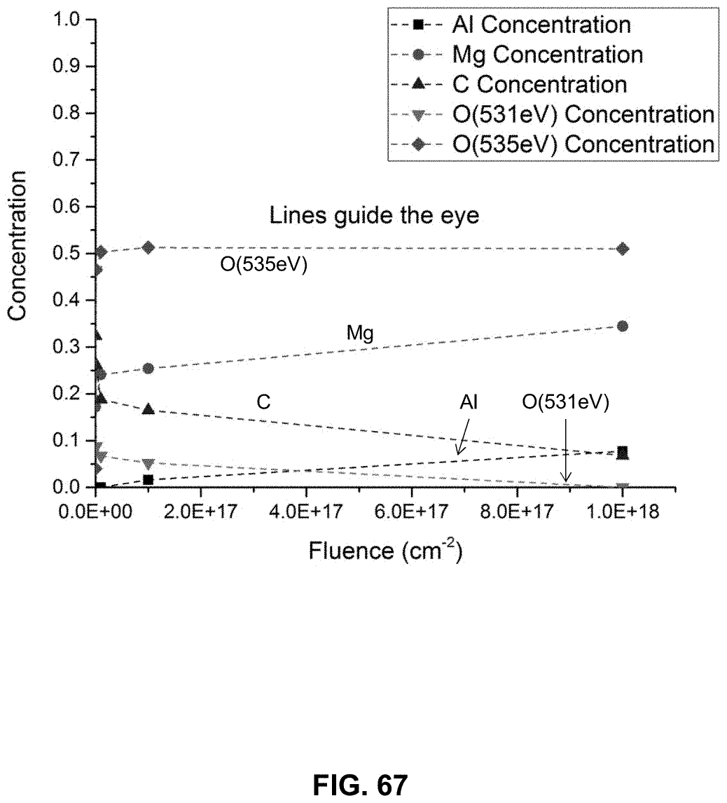

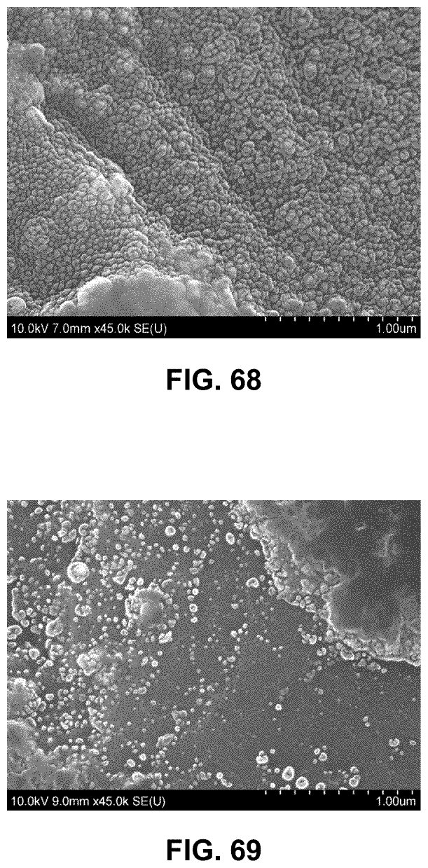

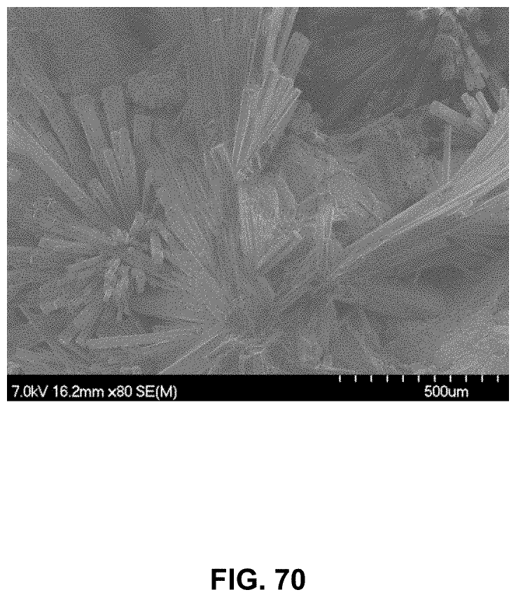

FIGS. 65-67 provide X-ray photoelectron spectroscopy data of a magnesium alloy sample surface treated with DPNS and illustrate the preferential increase of in-situ Al at the surface. The magnesium alloy sample is the Mg AZ 31 composition having 500 micron pores. The composition of Al is driven to about 10% at the modified surface when exposed to a fluence of 1×10¹⁸ particles per second per square centimeter as shown in FIG. 67. Additionally, the concentration of Zn (another component of the alloy) is not appreciably increased. The increase of Al while avoiding additional Zn at the modified surface improves hydroxyapatite formation and allows for precise control of corrosion rate.

FIG. 67 also illustrates that O1s at 531 eV concentration is decreased while O1s at 535 eV remains stable. This indicates that the MgO at the surface being modified is not decreasing while the Al is increasing. The data also suggests that Carbon decreases as impurities such as CO are removed from the modified surface.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A biodegradable sponge comprising:
a magnesium alloy comprising magnesium having an amount selected from the range of 96% to 97.3%, aluminum having an amount selected from the range of 3% to 3.5% and zinc having an amount selected from the range of 0.8% to 1.2%;
wherein said biodegradable sponge is characterized by a porosity selected over the range of 50% to 87%; and having Young's modulus selected from the range of 8 GPa to 29 GPa.

2. A biodegradable sponge comprising:
a magnesium alloy comprising magnesium having an amount selected from the range of 96% to 97.3%, aluminum having an amount selected from the range of 3% to 3.5% and zinc having an amount selected from the range of 0.8% to 1.2%;
wherein said biodegradable sponge is characterized by a porosity selected over the range of 50% to 87%; and having Young's modulus selected from the range of 8 GPa to 29 GPa;
said sponge having an outer exposed surface and an internal exposed surface provided by a plurality pores; wherein at least a portion of said exposed surface has a plurality of nanoscale domains providing a selected multifunctional bioactivity;
wherein said nanoscale domains are generated by exposing said surface to one or more directed energetic particle beam characterized by one or more beam properties.

3. The biodegradable sponge of claim 1 wherein said magnesium alloy further comprises one or more additional components selected from the group of potassium, calcium and manganese.

4. The biodegradable sponge of claim 1 wherein one or more additional components independently have an amount selected from the range of from the range of 0.01% to 0.05%.

5. The biodegradable sponge of claim 2, wherein said nanoscale domains comprise an increase or decrease in the aluminum content of said domain by greater than or equal to 10%.

6. The biodegradable sponge of claim 2, wherein said selected multifunctional bioactivity is with respect to an in vivo or in vitro activity relative to an unmodified magnesium containing sponge surface.

7. The biodegradable sponge of claim 5, wherein said in vivo or in vitro activity is a change in rate of bioresorption.

8. The biodegradable sponge of claim 7, wherein said change in rate of bioresorption is a decrease greater than or equal to a factor of 5.

9. The biodegradable sponge of claim 5, wherein said in vivo or in vitro activity is a decrease in hydrogen generation.

10. The biodegradable sponge of claim 9, wherein said decrease in hydrogen generation is a decrease greater than or equal to 10%.

11. The biodegradable sponge of claim 5, wherein said in vivo or in vitro activity is an enhancement in bioresorption, hydrogen generation, cell adhesion activity, cell shape activity, cell proliferation activity, cell migration activity, cell differentiation activity, anti-bacterial activity, bactericidal activity, anti-inflammatory activity, osseointegration activity, biocorrosion activity, cell differentiation activity, immuno-modulating activity during acute or chronic inflammation or any combination of these.

12. The biodegradable sponge of claim 11, wherein said enhancement of in vivo or in vitro activity is equal to or greater than 100%.

13. The biodegradable sponge of claim 2, wherein said nanoscale domains have an increased concentration of Aluminum.

14. The biodegradable sponge of claim 13, wherein said increased concentration of Aluminum promotes the formation of calcium phosphate when exposed to a fluid.

15. The biodegradable sponge of claim 2, wherein said nanoscale domains comprise an increase in Al₂O₃ content relative to the Al₂O₃ of an unmodified magnesium containing surface.

16. The biodegradable sponge of claim 2, wherein said nanoscale domains are provided between and within pores of said sponge to a depth of 540 μm from said external surface.

17. The biodegradable sponge of claim 2, wherein said nanoscale domains characterized by a chemical composition different from the bulk phase of said magnesium containing substrate.

18. The biodegradable sponge of claim 2, wherein said nanoscale domains provide an enhancement in vivo or in vitro activity with respect to cell adhesion proliferation activity and migration greater than or equal to 100%.

19. The biodegradable sponge of claim 2, wherein said nanoscale domains provide an enhancement in vivo or in vitro activity with respect to anti-bacterial activity and bactericidal activity greater than or equal to 100%.

20. The biodegradable sponge of claim 2, wherein said nanoscale domains provide local in vivo increase in pH, wherein said pH is increased by 0.5 or more.

21. The biodegradable sponge of claim 2, wherein said nanoscale domains provide an enhancement of a selected physical property of said substrate.

22. The biodegradable sponge of claim 21, wherein said physical property is hydrophilicity, hydrophobicity, surface free energy, surface charge density or any combination of these.

23. The biodegradable sponge of claim 21, wherein said enhancement of selected physical property is equal to or greater than 25%.

24. The biodegradable sponge of claim 1, wherein said biodegradable sponge is biocompatible.

25. The biodegradable sponge of claim 2, wherein the directed energetic particle beam is a broad beam, focused beam, asymmetric beam, reactive beam or any combination of these.

26. The biodegradable sponge of claim 2, wherein said one or more beam properties is intensity, fluence, energy, flux, incident angle, ion composition, neutral composition, ion to neutral ratio or any combinations thereof.

27. The biodegradable sponge of claim 2, wherein said nanoscale domains provide a surface geometry selected from the group consisting of topology, topography, morphology, texture or any combination of these.

28. The biodegradable sponge of claim 2, wherein each of said nanoscale domains are characterized by a vertical spatial dimension of less than or equal to 50 nm.

29. The biodegradable sponge of claim 2, wherein each of said nanoscale domains are characterized by a vertical spatial dimension selected over the range of 10 nm to 250 nm.

30. The biodegradable sponge of claim 2, wherein said nanoscale domains comprise nanowalls, nanorods, nanoplates, nanoripples or any combination thereof having lateral spatial dimensions selected over the range of 10 to 1000 nm and vertical spatial dimensions of less than or equal to 250 nm.

31. The biodegradable sponge of claim 30, wherein said nanowalls, nanorods, nanoplates or nanoripples are separated from one another by a distance of less than or equal to 100 nm.

32. The biodegradable sponge of claim 2, wherein said nanoscale domains comprise discrete crystallographic domains.

33. The biodegradable sponge of claim 1, wherein said biodegradable sponge is generated by infiltration casting and salt fluxing.

34. The biodegradable sponge of claim 1, wherein said biodegradable sponge has a tensile strength selected from the range of 5 MPa to 20 MPa.

35. A method of fabricating a biodegradable magnesium sponge comprising:
providing a magnesium containing sponge having a plurality of pores each having an surface; and
directing a directed energetic particle beam onto said surfaced, thereby generating a plurality of nanoscale domains on said surfaces;
wherein said directed energetic particle beam has one or more beam properties selected to generate said plurality of nanoscale domains providing a selected multifunctional bioactivity.

36. The method of claim 35, wherein the directed energetic particle beam is a broad beam, focused beam asymmetric beam or any combination of these.

37. The method of claim 35, wherein said step of directing said directed energetic particle beam onto said substrate surface comprises directed plasma nanosynthesis (DPNS), Direct Seeded Plasma Nanosynthesis (DSDPNS), Direct Soft Plasma Nanosynthesis (DSPNS) or any combination of these.

38. The method of claim 35, wherein said one or more beam properties is intensity, fluence, energy, flux, incident angle, ion composition, neutral composition ion to neutral ratio or any combinations thereof.

39. The method of claim 35, wherein said directed energetic particle beam comprises one or more ions, neutrals or combinations thereof.

40. The method of claim 39, wherein said ions are Ne ions, Kr ions, Ar ions, Xe ions, N ions or a combination thereof.

41. The method of claim 39, wherein said directed energetic particle beam is generated from an energetic 02 precursor.

42. The method of claim 35, wherein said one or more beam properties comprise incident angle and said incident angle is selected from the range of 0° to 80°.

43. The method of claim 35, wherein said one or more beam properties comprise fluence and said fluence is selected from the range of 1×10¹⁶ cm⁻² to 1×10¹⁹ cm⁻².

44. The method of claim 35, wherein said one or more beam properties comprise energy and said energy is selected from the range of 0.1 keV to 10 keV.

45. The method of claim 35, wherein said multifunctional bioactivity comprises bioresorption.
